(19) United States
(12) Patent Application Publication
(10) Pub. No.: US 2025/0000829 A1
(43) Pub. Date: Jan. 2, 2025

(54) PHENYLBUTYRATE COMPOSITIONS FOR TREATING DEVELOPMENTAL AND EPILEPTIC ENCEPHALOPATHY (“DEE”), AND STXBP1 DISORDERS

(71) Applicant: CORNELL UNIVERSITY, Ithaca, NY (US)

(72) Inventors: Zachary Grinspan, New York, NY (US); Jacqueline Burré, New York, NY (US)

(73) Assignee: CORNELL UNIVERSITY, Ithaca, NY (US)

(21) Appl. No.: 18/734,768

(22) Filed: Jun. 5, 2024

Related U.S. Application Data
(60) Provisional application No. 63/471,340, filed on Jun. 6, 2023.

(51) Int. Cl. A61K 31/192; A61P 25/00; A61P 25/08; C12Q 1/6883

(52) U.S. Cl. CPC A61K 31/192; A61P 25/00; A61P 25/08; C12Q 1/6883; C12Q 2600/106; C12Q 2600/156

(57) ABSTRACT

The present disclosure relates generally to methods for determining whether a patient suffering from or at risk for STXBP1- or SYNGAP1-encephalopathy will benefit from treatment with phenylbutyrate compositions comprising screening the patient for mutations in STXBP1 or SYNGAP1. Also disclosed herein are methods for selecting patients at risk for developmental and epileptic encephalopathy (DEE) for treatment with compositions including phenylbutyrate.

### CROSS REFERENCE TO RELATED APPLICATIONS

This application claims the benefit of and priority to U.S. Provisional Patent Application No. 63/471,340, filed on Jun. 6, 2023, the entirety of which is incorporated herein by reference.

### STATEMENT OF GOVERNMENT SUPPORT

This invention was made with government support under grant number 1R01NS102181 awarded by the National Institutes of Health. The government has certain rights in the invention.

### SEQUENCE LISTING

The instant application contains a Sequence Lising which has been submitted electronically in XML format and is hereby incorporated by reference in its entirety. Said XML copy, created on Aug. 29, 2024, is named 093873-1443_SL.xml and is 96,022 bytes in size.

### TECHNICAL FIELD

The present technology relates generally to methods for determining whether a patient suffering from or at risk for STXBP1 encephalopathy (STXBP1-E) will benefit from treatment with phenylbutyrate compositions comprising screening the patient for mutations in STXBP1 or SYNGAP1. Also disclosed herein are methods for selecting patients at risk for developmental and epileptic encephalopathy (DEE) for treatment with compositions including phenylbutyrate.

### BACKGROUND

The following description of the background of the present technology is provided simply as an aid in understanding the present technology and is not admitted to describe or constitute prior art to the present technology.

Epilepsy is a chronic neurologic disorder in which people have unprovoked seizures. People with epilepsy are at risk for injury from the seizures and have a higher rate of mortality compared to the general population. They have high rates of cognitive impairment, anxiety, depression, and social isolation. In children, epilepsy is often associated with other neurological problems such as developmental delay, cognitive impairment, learning disabilities, autism, cerebral palsy, and movement disorders. Treatment for epilepsy includes a variety of anti-seizure medication, with several dozen FDA approved currently available. Despite the range of available therapies, roughly one third of people with epilepsy continue to have seizures.

Accordingly, there is an urgent need to rapidly identify patients suffering from or at risk for developmental and epileptic encephalopathy (DEE) that are responsive to existing medications (e.g., phenylbutyrate).

### SUMMARY OF THE PRESENT TECHNOLOGY

In one aspect, the present disclosure provides a method for selecting a patient diagnosed with or at risk for STXBP1 encephalopathy (STXBP1-E) for treatment with a composition including phenylbutyrate or a pharmaceutically acceptable salt thereof comprising detecting the presence of a STXBP1 haploinsufficient allele in a biological sample obtained from the patient; and administering an effective amount of a composition including phenylbutyrate or a pharmaceutically acceptable salt thereof to the patient. In another aspect, the present disclosure provides a method for treating STXBP1 encephalopathy (STXBP1-E) in a patient in need thereof comprising administering an effective amount of a composition including phenylbutyrate or a pharmaceutically acceptable salt thereof to the patient, wherein the patient comprises a STXBP1 haploinsufficient allele.

In one aspect, the present disclosure provides a method for selecting a patient diagnosed with or at risk for SYNGAP1 encephalopathy for treatment with a composition including phenylbutyrate or a pharmaceutically acceptable salt thereof comprising detecting the presence of a SYNGAP1 haploinsufficient allele in a biological sample obtained from the patient; and administering an effective amount of a composition including phenylbutyrate or a pharmaceutically acceptable salt thereof to the patient. In another aspect, the present disclosure provides a method for treating SYNGAP1 encephalopathy in a patient in need thereof comprising administering an effective amount of a composition including phenylbutyrate or a pharmaceutically acceptable salt thereof to the patient, wherein the patient comprises a SYNGAP1 haploinsufficient allele

The STXBP1 or SYNGAP1 haploinsufficient allele may comprise a nonsense mutation, a missense mutation, a deletion, an inversion or a frameshift mutation. In certain embodiments, the STXBP1 haploinsufficient allele is S311FfsX3, R292P, or R190W. In some embodiments, the SYNGAP1 haploinsufficient allele is R579X, K1185*, R621*, Y928*, or C576*. Additionally or alternatively, in some embodiments, the STXBP1 or SYNGAP1 haploinsufficient allele is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). In any and all of the preceding embodiments, the composition comprises glycerol phenylbutyrate.

Additionally or alternatively, in certain embodiments, the patient does not comprise a dominant-negative STXBP1 mutation, optionally wherein the dominant-negative STXBP1 mutation is selected from the group consisting of V84D, C180Y, L183R, P335L, R406C, R406H, M443R, P480L, G544D, G544V, R551C, C552R, and T574R. In some embodiments, the patient does not comprise one or more STXBP1 mutations selected from among P139L, E283K, and Q229*. In some embodiments, the patient does not comprise one or more SYNGAP1 mutations selected from among S190Cysfs*2 or S225HfsX26. In some embodiments, the patient does not comprise an IRF2BPL mutation comprising A101ThrfsTer18.

In any of the preceding embodiments of the methods disclosed herein, the STXBP1-E presents as infantile spasms, epilepsy of infancy migrating focal seizure, Dravet syndrome or non-syndromic epilepsy.

Additionally or alternatively, in certain embodiments, the patient does not exhibit active epileptic spasms. In any and all embodiments of the methods disclosed herein, the patient exhibits one or more of autism, low tone, movement disorders (including ataxia or bruxism), abnormal EEGs (>60% with focal or multifocal epileptiform discharges), or abnormal MRI brain imaging (including atrophy, thin corpus callosum, or delayed myelination).

In one aspect, the present disclosure provides a method for treating developmental and epileptic encephalopathy (DEE) in a patient in need thereof comprising administering an effective amount of a composition including phenylbutyrate or a pharmaceutically acceptable salt thereof to the patient, wherein the patient does not exhibit active epileptic spasms. The patient may comprise a STXBP1 haploinsufficient allele, a SYNGAP1 haploinsufficient allele, and/or a SLC6A1 haploinsufficient allele. Additionally or alternatively, in some embodiments, the STXBP1 haploinsufficient allele, SYNGAP1 haploinsufficient allele, or SLC6A1 haploinsufficient allele comprises a nonsense mutation, a missense mutation, a deletion, an inversion or a frameshift mutation. Examples of STXBP1 haploinsufficient alleles include, but are not limited to, S311FfsX3, R292P, or R190W. Examples of SYNGAP1 haploinsufficient allelles include, but are not limited to, R579X, K1185*, R621*, Y928Ter, or C576*. Examples of SLC6A1 haploinsufficient alleles include, but are not limited to D52N, L408WfsX26, A288V, G297R, A305T, V125M, S295L, G362R, D410E, L460R and W495X.

Additionally or alternatively, in certain embodiments, the patient does not comprise one or more STXBP1 mutations selected from the group consisting of V84D, C180Y, L183R, P335L, R406C, R406H, M443R, P480L, G544D, G544V, R551C, C552R, T574R, P139L, E283K, and Q229*. In some embodiments, the patient does not comprise one or more SYNGAP1 mutations selected from among S190Cysfs*2 or S225HfsX26. In some embodiments, the patient does not comprise an IRF2BPL mutation comprising A101ThrfsTer18. In any and all of the preceding embodiments, the composition comprises glycerol phenylbutyrate.

In any and all embodiments of the methods disclosed herein, the patient is an infant, a child or an adolescent. Additionally or alternatively, in some embodiments of the methods disclosed herein, the effective amount of the composition including phenylbutyrate or the pharmaceutically acceptable salt thereof is about 5 g/m²/day to about 12.5 g/m²/day.

In any of the preceding embodiments of the methods disclosed herein, the composition including phenylbutyrate or the pharmaceutically acceptable salt thereof is administered enterally, optionally wherein the composition is administered via a nasogastric or gastrostomy tube. Additionally or alternatively, in some embodiments of the methods disclosed herein, the composition is administered daily for at least 6 weeks, optionally wherein the composition is administered thrice a day.

In any and all embodiments of the methods disclosed herein, administration of the composition results in amelioration of one or more of reduction in seizures, improvement in cognition, reduction in abnormal movement, improvement in sleep quality, improved gait, improved quality life, improved mood, reduction in bruxism, improvement in gross motor skills, improvement in fine motor skills, improvement in communication skills, or reduction in family burden. Additionally or alternatively, the methods disclosed herein further comprise administering to the patient one or more of steroids, vigabatrin, or a ketogenic diet.

### BRIEF DESCRIPTION OF THE DRAWINGS

FIGS. 1A-1C show disease-linked missense mutations in protein structures. FIG. 1A shows disease-linked missense mutations in STXBP1, including its primary sequence with indication of domain structures and positions of mutations (top) and its three-dimensional (3D) structure with mutated residues highlighted (bottom). FIG. 1B shows disease-linked missense mutations in Munc18-1 (a.k.a. STXBP1), including its primary sequence with indication of domain structures and positions of mutations (top) and its 3D structure with (left) and without (right) annotation of residues. FIG. 1C shows a 3D structure of STXBP1 with disease-linked missense mutations highlighted in patients with DEE.

FIG. 2 is an enrollment checklist for enrollment in the study measuring the effectiveness of glycerol phenylbutyrate to treat patients with STXBP1-E and SLC6A1-NDD, as described in the Examples section below.

FIG. 3 is a schedule of assessments for measuring the effectiveness of glycerol phenylbutyrate to treat patients with STXBP1-E and SLC6A1-NDD, as described in the Examples section below.

FIGS. 4A-4I are example questionnaires used as assessments in studies measuring the effectiveness of glycerol phenylbutyrate to treat patients with STXBP1-E and SLC6A1-NDD, as described in the Examples section below. FIG. 4A is a seizure diary. FIG. 4B is a Pediatric Epilepsy Learning Healthcare System Quality of Life (PELHS-QOL-2) questionnaire. FIGS. 4C-4E show the child's sleep habits questionnaire. FIGS. 4F-4G show the scale for the assessment and rating of ataxia (SARA). FIG. 4H is a side effect questionnaire. FIG. 4I is a medication diary.

FIG. 5 is a summary of the results of the clinical trial described in the Examples described herein.

FIG. 6 show electroencephalograms prior to treatment (top) and six weeks after treatment with glycerol phenylbutyrate (bottom).

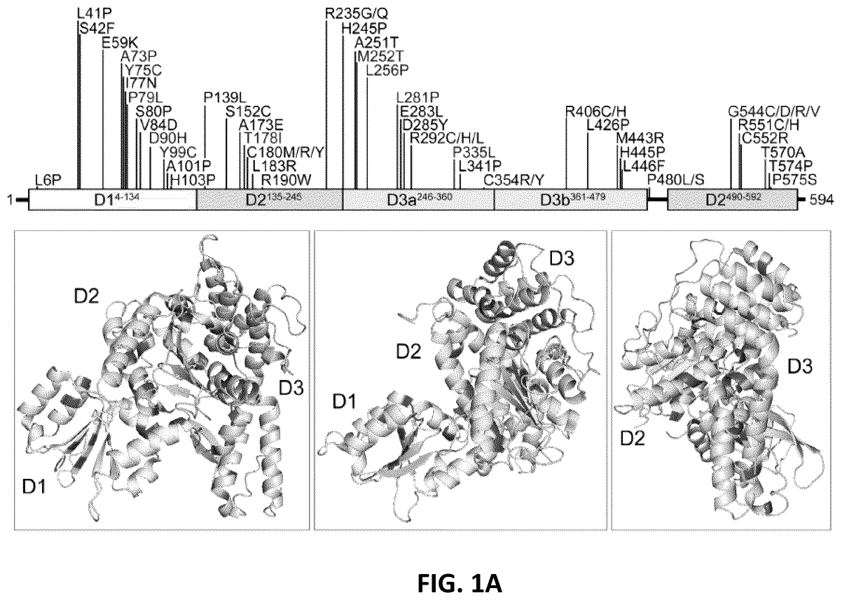

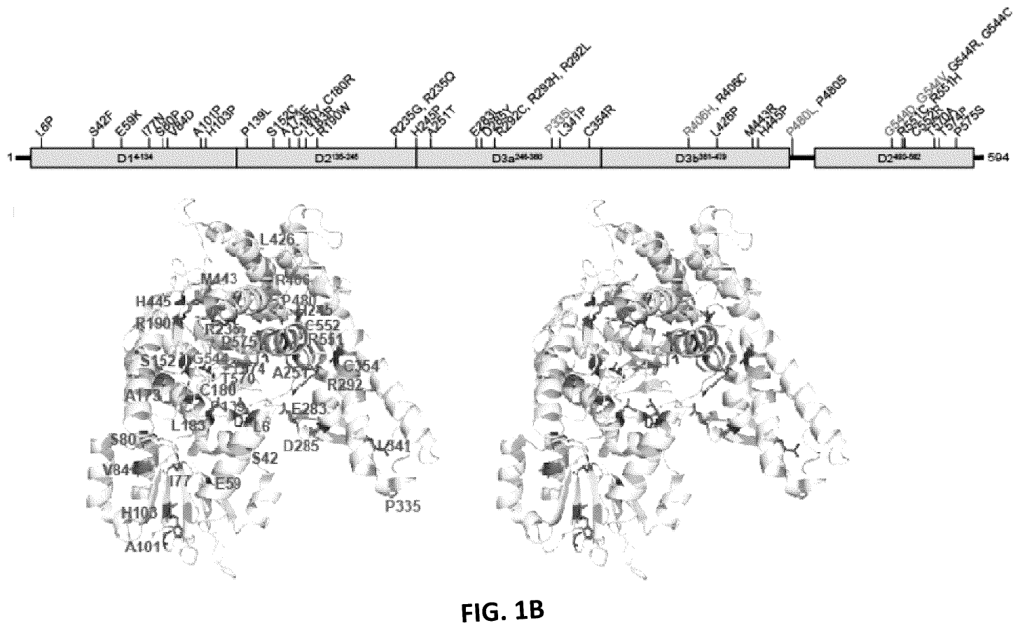

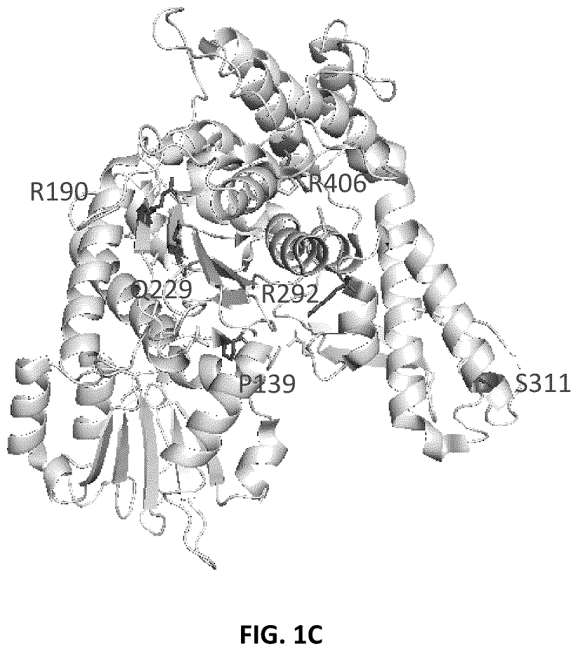

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Historically, STXBP1-related disorders are considered to present without clear genotype-phenotype correlations. Surprisingly, none of the major recurrent variants in STXBP1 showed significant phenotypic similarity, in contrast to the main recurrent variants in other neurodevelopmental disorders such as SCN2A-related disorders. Xian et al., Brain, Volume 145, Issue 5, Pages 1668-1683 (2022). This lack of phenotypic similarity for recurrent variants indicates that when compared to the phenotypes of STXBP1-disorders as a whole, the baseline variability of phenotypic features is too high for single-phenotype associations to allow for discrete clinical subgroups to be recognized. When excluding the major recurrent variants, no domain of the STXBP1 protein was associated with specific phenotypic features. Thus, prior studies have established that any variant-specific clinical homogeneity was too subtle to distinguish from the overall heterogeneity in STXBP1-related disorders, and overall phenotypic spectrum of STXBP1-related disorders is broad, including a wide range of epilepsy presentations and neurological features. Accordingly, there are no existing methods that can accurately and reliably discriminate between responders and non-responders to phenylbutyrate in DEE patient populations.

The present disclosure demonstrates that the methods of the present technology can be used to effectively identify DEE patients that are responsive to phenylbutyrate therapy.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

“Active epileptic spasms” as used herein refers to a subject that has had seizures of the subtype “epileptic spasms” within the preceding one week. An epileptic spasm is typically a brief flexor movement of the arms, head, legs, and/or trunk that is sustained for 1-2 seconds. If EEG is recorded simultaneously, there is a characteristic “electro decrement” pattern associated with the epileptic spasm. In some instances, epileptic spasms occur in clusters, 10-30 seconds apart, over 5-8 minutes.

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, the “administration” of an agent or drug to a subject includes any route of introducing or delivering to a subject a compound to perform its intended function. Administration can be carried out by any suitable route, including but not limited to, orally, enterally, intranasally, parenterally (intravenously, intramuscularly, intraperitoneally, or subcutaneously), rectally, intrathecally, or topically. Administration includes self-administration and the administration by another.

As used herein, the terms “amplify” or “amplification” with respect to nucleic acid sequences, refer to methods that increase the representation of a population of nucleic acid sequences in a sample. Nucleic acid amplification methods are well known to the skilled artisan and include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA) (TwistDx, Cambridge, UK), transcription mediated amplification, signal mediated amplification of RNA technology, loop-mediated isothermal amplification of DNA, helicase-dependent amplification, single primer isothermal amplification, and self-sustained sequence replication (3SR), including multiplex versions or combinations thereof. Copies of a particular nucleic acid sequence generated in vitro in an amplification reaction are called “amplicons” or “amplification products.”

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state). In other embodiments, the reference nucleic acid sample is from a healthy control subject that is not at risk for DEE.

“Detecting” as used herein refers to determining the presence of a mutation or alteration in a nucleic acid of interest in a sample. Detection does not require the method to provide 100% sensitivity. Analysis of nucleic acid markers can be performed using techniques known in the art including, but not limited to, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol, 3:39-42 (1992)), sequencing with mass spectrometry such as matrix-assisted laser desorption/ionization time-of-flight mass spectrometry (MALDI-TOF/MS; Fu et al., Nat. Biotechnol, 16:381-384 (1998)), and sequencing by hybridization. Chee et al., Science, 274:610-614 (1996); Drmanac et al., Science, 260:1649-1652 (1993); Drmanac et al., Nat. Biotechnol, 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

“Detectable label” as used herein refers to a molecule or a compound or a group of molecules or a group of compounds used to identify a nucleic acid or protein of interest. In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable labels include but are not limited to spectroscopic, photochemical, biochemical, immunochemical, electromagnetic, radiochemical, or chemical means, such as fluorescence, chemifluorescence, or chemiluminescence, or any other appropriate means.

As used herein, “developmental and epileptic encephalopathy” or “DEE” is a condition in children in which there is epilepsy as well as developmental delay. DEEs are often monogenetic disorders and may be caused by mutations in genes such as STXBP1, SYNGAP1, and SLC6A1.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression. Specifically, a gene refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, “haploinsufficiency” refers to a condition in which a single copy of the wild-type allele at a locus in heterozygous combination with a variant allele is insufficient to produce the wild-type phenotype, but yields a phenotype that is less severe compared to the homozygous variant allele at the same locus. Haploinsufficiency may arise from a de novo or inherited loss-of-function mutation in the variant allele, such that it yields little or no gene product. Although the other, standard allele still produces the standard amount of product, the total product is insufficient to produce the standard phenotype. This heterozygous genotype may result in a non- or sub-standard, deleterious, and (or) disease phenotype.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” can be an individual organism, a vertebrate, a mammal, or a human. In some embodiments, the individual, patient or subject is a human.

As used herein, the term “library” refers to a collection of nucleic acid sequences, e.g., a collection of nucleic acids derived from whole genomic, subgenomic fragments, cDNA, cDNA fragments, RNA, RNA fragments, or a combination thereof. In one embodiment, a portion or all of the library nucleic acid sequences comprises an adapter sequence. The adapter sequence can be located at one or both ends. The adapter sequence can be useful, e.g., for a sequencing method (e.g., an NGS method), for amplification, for reverse transcription, or for cloning into a vector.

The library can comprise a collection of nucleic acid sequences, e.g., a target nucleic acid sequence (e.g., a STXBP1 or SYNGAP1 mutant nucleic acid sequence), a reference nucleic acid sequence, or a combination thereof. In some embodiments, the nucleic acid sequences of the library can be derived from a single subject. In other embodiments, a library can comprise nucleic acid sequences from more than one subject (e.g., 2, 3, 4, 5, 6, 7, 8, 9, 10, 20, 30 or more subjects). In some embodiments, two or more libraries from different subjects can be combined to form a library having nucleic acid sequences from more than one subject.

A “library nucleic acid sequence” refers to a nucleic acid molecule, e.g., a DNA, RNA, or a combination thereof, that is a member of a library. Typically, a library nucleic acid sequence is a DNA molecule, e.g., genomic DNA or cDNA. In some embodiments, a library nucleic acid sequence is fragmented, e.g., sheared or enzymatically prepared, genomic DNA. In certain embodiments, the library nucleic acid sequences comprise sequence from a subject and sequence not derived from the subject, e.g., adapter sequence, a primer sequence, or other sequences that allow for identification, e.g., “barcode” sequences.

The term “multiplex PCR” as used herein refers to amplification of two or more PCR products or amplicons which are each primed using a distinct primer pair.

As used herein, a “mutation” of a gene or gene product (e.g., a marker gene or gene product) refers to the presence of an alteration or alterations within the gene or gene product, which affects the quantity or activity of the gene or gene product, as compared to the normal or wild-type gene. The genetic alteration can result in changes in the quantity, structure, and/or activity of the gene or gene product in a tissue or cell of a DEE subject, as compared to its quantity, structure, and/or activity, in a normal or healthy tissue or cell (e.g., a control). For example, a mutation which is associated with DEE, or predictive of responsiveness to phenylbutyrate, can have an altered nucleotide sequence, amino acid sequence, chromosomal translocation, intra-chromosomal inversion, copy number, expression level, protein level, protein activity, in a tissue or cell of the subject, as compared to a normal, healthy tissue or cell. Exemplary mutations include, but are not limited to, point mutations (e.g., silent, missense, or nonsense), deletions, insertions, inversions, linking mutations, duplications, translocations, inter- and intra-chromosomal rearrangements. Mutations can be present in the coding or non-coding region of the gene.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the active agent (e.g., phenylbutyrate), e.g., C₁₋₆ alkyl esters. When there are two acidic groups present, a pharmaceutically-acceptable salt or ester can be a mono-acid-mono-salt or ester or a di-salt or ester; and similarly where there are more than two acidic groups present, some or all of such groups can be salified or esterified.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a target nucleic acid. Probes are typically at least about 10, 15, 20, 25, 30, 35, 40, 50, 60, 75, 100 nucleotides or more in length.

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample.

As used herein, a “sample” refers to a substance that is being assayed for the presence of a mutation in a nucleic acid of interest. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%).

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity.

“Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not true} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include 90, 95, 98, and 99%.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “target sequence” and “target nucleic acid sequence” refer to a specific nucleic acid sequence to be detected and/or quantified in the sample to be analyzed.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### STXBP1, SYNGAP1 and SLC6A1-Related Disorders

Generalized onset seizures affect both sides of the brain or groups of cells on both sides of the brain at the same time and includes seizures types like tonic-clonic, absence, myoclonic, or atonic. For generalized onset seizures, motor symptoms may include sustained rhythmical jerking movements (clonic), muscles becoming weak or limp (atonic), muscles becoming tense or rigid (tonic), brief muscle twitching (myoclonus), or epileptic spasms (body flexes and extends repeatedly). Non-motor symptoms are usually called absence seizures, which can be brief typical or prolonged atypical absence seizures (staring spells). Absence seizures can also have brief twitches (myoclonus) that can affect a specific part of the body or just the eyelids.

For focal onset seizures, motor symptoms may also include jerking (clonic), muscles becoming limp or weak (atonic), tense or rigid muscles (tonic), brief muscle twitching (myoclonus), or epileptic spasms. There may also be automatisms or repeated automatic movements, like clapping or rubbing of hands, lipsmacking or chewing, or running. Non-motor symptoms that don't affect movement could be changes in sensation, emotions, thinking or cognition, autonomic functions (such as gastrointestinal sensations, waves of heat or cold, goosebumps, heart racing, etc.), or lack of movement (called behavior arrest).

STXBP1 encephalopathy (STXBP1-E) is a devastating neurodevelopmental disorder that often begins in infancy. Intellectual disability is a core feature, often severe to profound. Nearly all have epilepsy (95% in the largest series). The epilepsy is clinically heterogeneous, and may present as a well-defined epilepsy syndrome (e.g., early infantile epileptic encephalopathy, infantile spasms, epilepsy of infancy migrating focal seizure, or Dravet syndrome) or as non-syndromic epilepsy. Seizures are refractory to medications in one third. Affected individuals may have autistic features (1 in 5), low tone, movement disorders (including ataxia and bruxism6), abnormal EEGs (>60% with focal or multifocal epileptiform discharges), and/or abnormal MRI brain imaging (atrophy, thin corpus callosum, delayed myelination). The clinical spectrum is broad—some individuals are profoundly impaired with seizures that begin in the first days of life; whereas others may have a few seizures in late infancy and mild learning difficulties.

STXBP1 knockout mice show normal early brain assembly with subsequent degeneration, decreased neurite outgrowth, and completely abolished neurotransmitter release. These mice die shortly after birth. Heterozygous STXBP1 mice are similar to wild-type, except they have abnormal behaviors during sleep (twitches and jumps) and EEG abnormalities. Thus, heterozygous STXBP1 mice recapitulate some aspects of the human disease, though they have neither seizures nor overt behavioral abnormalities. Published experiments on neuronal lines derived from affected patients show decreased STXBP1 protein, STXBP1 protein mislocalization, and decreased neurite outgrowth. Early work in heterologous cell lines demonstrated STXBP1 mutations cause protein misfolding that leads to aggregation of the mutant protein with wild-type STXBP1.

SLC6A1-related neurodevelopmental disorder (SLC6A1-NDD) begins in early childhood and is characterized by epilepsy (˜91%, typically generalized) and developmental delay (˜82%). The epilepsy is typically generalized (absence, atonic, myoclonic, generalized tonic-clonic) though is sometimes focal. Substantial minorities have an autism spectrum disorder, movement disorder, or problems with attention or aggression. The protein product of SLC6A1 is GABA transporter protein type 1 (GAT-1), which is important for GABA homeostasis in the brain. Pathogenic mutations in SLC6A1 lead to loss of function and haploinsufficiency. Human Embryonic Kidney (HEK) cells transfected with 17 human variants of GAT-1 cDNA all demonstrated a significant reduction in GABA uptake. The data suggest that the pathology of these variants may originate from shifts in residue hydropathy as well as the location of the variant across the membrane.

A distinctive generalized DEE phenotype for SYNGAP1 combines syndromic features of epilepsy with eyelid myoclonia with absences (EMA) and epilepsy with myoclonic-atonic seizures (MAE). SYNGAP1 encephalopathy is associated with a spectrum of mild to severe intellectual disability and a range of other comorbid conditions. Seizures are often triggered by eating. SYNGAP1-DEE shares many of the striking features of the syndrome of EMA. The key seizure type, absences with EM, together with photosensitivity, was found in the majority (64% and 55%, respectively) of affected patients. The onset of EM was earlier for SYNGAP1-DEE (<3 years) compared with the classic syndrome of EMA (peak onset 6-8 years). In addition, EMA is often associated with better cognitive outcome, with individuals being of normal intellect or having mild ID. An earlier age at onset (<3 years) of EM is associated with poorer intellectual outcome, and perhaps some of these individuals have SYNGAP1 mutations.

Methods for Detecting Polynucleotides Associated with Responsiveness to Phenylbutyrate or Pharmaceutically Acceptable Salts Thereof

Polynucleotides associated with responsiveness to compositions including phenylbutyrate or a pharmaceutically acceptable salt thereof may be detected by a variety of methods known in the art. Non-limiting examples of detection methods are described below. The detection assays in the methods of the present technology may include purified or isolated DNA (genomic or cDNA), RNA or protein or the detection step may be performed directly from a biological sample without the need for further DNA, RNA or protein purification/isolation.

### Nucleic Acid Amplification and or Detection

Polynucleotides associated with responsiveness to compositions including phenylbutyrate or a pharmaceutically acceptable salt thereof can be detected by the use of nucleic acid amplification techniques that are well known in the art. The starting material may be genomic DNA, cDNA, RNA, or mRNA. Nucleic acid amplification can be linear or exponential. Specific variants or mutations may be detected by the use of amplification methods with the aid of oligonucleotide primers or probes designed to interact with or hybridize to a particular target sequence in a specific manner, thus amplifying only the target variant.

Non-limiting examples of nucleic acid amplification techniques include polymerase chain reaction (PCR), real-time quantitative PCR (qPCR), digital PCR (dPCR), reverse transcriptase polymerase chain reaction (RT-PCR), nested PCR, ligase chain reaction (see Abravaya, K. et al., Nucleic Acids Res. (1995), 23:675-682), branched DNA signal amplification (see Urdea, M. S. et al., AIDS (1993), 7 (suppl 2): S11-S14), amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, next-generation sequencing technology or other sequence replication assays or signal amplification assays.

Primers: Oligonucleotide primers for use in amplification methods can be designed according to general guidance well known in the art as described herein, as well as with specific requirements as described herein for each step of the particular methods described. In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length.

Tₘ of a polynucleotide affects its hybridization to another polynucleotide (e.g., the annealing of an oligonucleotide primer to a template polynucleotide). In certain embodiments of the disclosed methods, the oligonucleotide primer used in various steps selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In certain embodiments, 100% complementarity exists.

Probes: Probes are capable of hybridizing to at least a portion of the nucleic acid of interest or a reference nucleic acid (i.e., wild-type sequence). Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, or about 100 nucleotides long. However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, or about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif, 1987); Young and Davis, PNAS. 80:1194 (1983).

Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29: E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Detectably labeled probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detectably labeled probes can also include two probes, wherein for example a fluorophore is on one probe, and a quencher is on the other probe, wherein hybridization of the two probes together on a target quenches the signal, or wherein hybridization on the target alters the signal signature via a change in fluorescence.

In some embodiments, interchelating labels such as ethidium bromide, SYBR® Green I (Molecular Probes), and PicoGreen® (Molecular Probes) are used, thereby allowing visualization in real-time, or at the end point, of an amplification product in the absence of a detector probe. In some embodiments, real-time visualization may involve the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

Primers or probes may be designed to selectively hybridize to any portion of a nucleic acid sequence encoding a polypeptide selected from among STXBP1, SYNGAP1, and/or SLC6A1. Exemplary nucleic acid sequences of the human orthologs of these genes are provided below:
NM_001032221.6 Homo sapiens syntaxin binding protein 1 (STXBP1), transcript variant 2, mRNA (SEQ ID NO: 1)

AGTCGGTCCCTAGCGCGGCTGCGGGGCGGAGAGCTGCGGCTGGCCCAGCG
CGCCCACCTGAGGAGGCGGCGGGGTCCGCAGGCGTCGCGGGACGAGGAGA
TCGGAGCCGGGAGACTCGCGCAGCGCCATGGCCCCCATTGGCCTCAAAGC
TGTTGTCGGAGAGAAGATTATGCATGATGTGATAAAGAAGGTCAAGAAGA
AGGGGGAATGGAAGGTGCTGGTGGTGGATCAGTTAAGCATGAGGATGCTG
TCCTCCTGCTGCAAGATGACAGACATCATGACCGAGGGCATAACGATTGT
GGAAGATATCAATAAGCGCAGAGAGCCGCTCCCCAGCCTGGAGGCTGTGT
ATCTCATCACTCCATCCGAGAAGTCCGTCCACTCTCTCATCAGTGACTTT
AAGGACCCGCCGACTGCTAAATACCGGGCTGCACACGTCTTCTTCACTGA
CTCTTGTCCAGATGCCCTGTTTAATGAACTGGTAAAATCCCGAGCAGCCA
AAGTCATCAAAACTCTGACGGAAATCAATATTGCATTTCTCCCGTATGAA
TCCCAGGTCTATTCCTTGGACTCTGCTGACTCTTTCCAAAGCTTCTACAG
TCCCCACAAGGCTCAGATGAAGAATCCTATACTGGAGCGCCTGGCAGAGC
AGATCGCGACCCTTTGTGCCACCCTGAAGGAGTACCCGGCTGTGCGGTAT
CGGGGGGAATACAAGGACAATGCCCTGCTGGCTCAGCTAATCCAGGACAA
GCTCGATGCCTATAAAGCTGATGATCCAACAATGGGGGAGGGCCCAGACA
AGGCACGCTCCCAGCTCCTGATCCTGGATCGAGGCTTTGACCCCAGCTCC
CCTGTGCTCCATGAATTGACTTTTCAGGCTATGAGTTATGATCTGCTGCC
TATCGAAAATGATGTATACAAGTATGAGACCAGCGGCATCGGGGAGGCAC
GGGTGAAGGAGGTGCTCCTGGACGAGGACGACGACCTGTGGATAGCACTG
CGCCACAAGCACATCGCAGAGGTGTCCCAGGAAGTCACCCGGTCTCTGAA
AGATTTTTCTTCTAGCAAGAGAATGAATACTGGAGAGAAGACCACCATGC
GGGACCTGTCCCAGATGCTGAAGAAGATGCCTCAGTACCAGAAAGAGCTC
AGCAAGTACTCCACCCACCTGCACCTTGCTGAGGACTGTATGAAGCATTA
CCAAGGCACCGTAGACAAACTCTGCCGAGTGGAGCAGGACCTGGCCATGG
GCACAGATGCTGAGGGAGAGAAGATCAAGGACCCTATGCGAGCCATCGTC
CCCATTCTGCTGGATGCCAATGTCAGCACTTATGACAAAATCCGCATCAT
CCTTCTCTACATCTTTTTGAAGAATGGCATCACGGAGGAAAACCTGAACA
AACTGATCCAGCACGCCCAGATACCCCCGGAGGATAGTGAGATCATCACC
AACATGGCTCACCTCGGCGTGCCCATCGTCACCGATTCCACGCTGCGTCG
CCGGAGCAAGCCGGAGCGGAAGGAACGCATCAGCGAGCAGACCTACCAGC
TCTCACGGTGGACTCCGATTATCAAGGACATCATGGAGGACACTATTGAG
GACAAACTTGACACCAAACACTACCCTTATATCTCTACCCGTTCCTCTGC
CTCCTTCAGCACCACCGCCGTCAGCGCCCGCTATGGGCACTGGCATAAGA
ACAAGGCCCCAGGCGAGTACCGCAGTGGCCCCCGCCTCATCATTTTCATC
CTTGGGGGTGTGAGCCTGAATGAGATGCGCTGCGCCTACGAGGTGACCCA
GGCCAACGGAAAGTGGGAGGTGCTGATAGGATCCACACACATCCTCACCC
CACAGAAACTGCTGGACACACTGAAGAAACTGAATAAAACAGATGAAGAA
ATAAGCAGTTAAAAAAATAAGTCGCCCCTCCAAAACACGCCCCCATCCCA
CAGCGCTCCGCAGCTTCCCACCACCGCCCGCCTCAGTTCCTTTGCGTCTG
TTGCCTCCCCAGCCCTGCACGCCCTGGCTGGCACTGTTGCCGCTGCATTC
TCGTGTTCAGTGATGCCCTCTTCTTGTTTGAAACAAAAGAAAATAATGCA
TTGTGTTTTTTAAAAAGAGTATCTTATACATGTATCCTAAAAAGAGAAGC
TCATGTGCAATTGGTGCACAGCAGGAGAAATTTCTGGACTGTTAGGATGA
ATGGACGCCTTCTCCCCGTTATTTAAGATTTGTGACCTTGTACATAACCC
TGGGTGACGTGCACATTGCTTGGGTATGGAACGGTAGAAATTTGGGTGTT
TTTAAAACCTTGTTTGGGGTTGTTCCTGTCCTTGTTGAGAATCATAGAGA
TGTCTGTGTTCTTGGAGTATTTCACACTGAGGACTAATCTGCTATCTTCA
TTCCAGTCCCTACCCCTCAGTGCCTGCTCTCATCCAAATAACCTGGGAGG
TGACAATCAGGATATCTCAGGAGGTCCAAGGTGGAACAGACCTCTTTGCC
TTTCCCAGCGTCTCATACCCCCGGTAGTGCAGCTGTGGGTGGAGGCTGGG
GTGTCTGCACGAAGTCAGGCCAGCGTCCTCCTCCACAGCCTGTCACTGCC
CCCTCCCCAGCCTGTGTCCACAGTGCTGTGATCCCGAGGGAAGTCCTCCA
GTCTAAGTCACAGTGCCCTGACAGGTGAGAAGCAAACTCCCGCTGGAAGC
CTCCATCTCTTTGGAAAAACAGTTAGTCTGGAGCCTGTGGCCCAGGCCCT
TCTGTCCCCAGGCATCATCCCAACAGCTCATTTTCCCTAGTCCGCCTTCG
TTCAAGGGTCAGGAATGGACCAGAACAGATGGGTTCTGGAGGCCCCTGAA
CAGAGGGCTATGGCTGTGGAGAAGGTTCTTGGCCCGTTGGACTCACACAG
ACCCTGTACCCTCTCGGCAAGCATCTTCAGTCAGATTATCCTCAGTTTCA
GATACTTCATAATACCTTGTGTTGTGTGGGGTCATACATCATCGTGTTTG
TAAGAGAAGATGGTCATTTTATTCTCTGTATAAAACTTAGCTCTAAAGCA
GAAACTAAAGCAGCAAATGCAGGAAGGCTGTCTCGCCATCCTCAAGACTC
AGCAGCTCTCATTCTCCAGTGGTGAGCACACCATTTGTGCTGCTGCTGTT
GTCGTGAAATATAATAACAGTGGAAGTCACAAAAATGTCCCCTGCCCAGC
CCCCTCGCCGCCCTTGACCTCCTGCAGGCCATGTGTGTATTACTTGTCTA
GTGATGTCCTCTCAAAGTGCTGTACGCGAGCTCGGCGCCACCTCCGCCTC
CCTTTCAGAGCCTGCTCCCCGCCCTCTCTGCTCGCTGCATTGTGGTGTTC
TCTTCTCAAGGCTTTGAAATCTCCCCTTGCACTGAGATTAGTCGTCAGAT
CTCTCCCCGTCTCCCTCCCAACTTATACGACCTGATTTCCTTAGGACGGA
ACCGCAGGCACCTGCGCCGGGCGTCTTACTCCCGCTGCTTGTTCTGTCCC
CTCCCTCGGACCAAACAGTGCTCATGCTTCAGGACCTTGTTTGTCGAAGA
TGTTGGTTTCCCTTTCTCTGTTATTTATATAAAAATAATTTATCAAAAGG
ATATTTTAAAAAAGCTAGTCTGTCTTGAAACTTGTTTACCTTAAAATTAT
CAGAATCTCAGTGTTTGAAAGTACTGAAGCACAAACATATATCATCTCTG
TACCATTCTGTACTAAAGCACTTGAGTCTAATAAATAAAGAAATCAGCAC
CCCT

NM 001374306.2 Homo sapiens syntaxin binding protein 1 (STXBP1), transcript variant 3, mRNA (SEQ ID NO: 2)

AGTCGGTCCCTAGCGCGGCTGCGGGGCGGAGAGCTGCGGCTGGCCCAGCG
CGCCCACCTGAGGAGGCGGCGGGGTCCGCAGGCGTCGCGGGACGAGGAGA
TCGGAGCCGGGAGACTCGCGCAGCGCCATGGCCCCCATTGGCCTCAAAGC
TGTTGTCGGAGAGAAGATTATGCATGATGTGATAAAGAAGGTCAAGAAGA
AGGGGGAATGGAAGGTGCTGGTGGTGGATCAGTTAAGCATGAGGATGCTG
TCCTCCTGCTGCAAGATGACAGACATCATGACCGAGGGCATAACGATTGT
GGAAGATATCAATAAGCGCAGAGAGCCGCTCCCCAGCCTGGAGGCTGTGT
ATCTCATCACTCCATCCGAGAAGTCCGTCCACTCTCTCATCAGTGACTTT
AAGGACCCGCCGACTGCTAAATACCGGGCTGCACACGTCTTCTTCACTGA
CTATGCCCTGTTTAATGAACTGGTAAAATCCCGAGCAGCCAAAGTCATCA
AAACTCTGACGGAAATCAATATTGCATTTCTCCCGTATGAATCCCAGGTC
TATTCCTTGGACTCTGCTGACTCTTTCCAAAGCTTCTACAGTCCCCACAA
GGCTCAGATGAAGAATCCTATACTGGAGCGCCTGGCAGAGCAGATCGCGA
CCCTTTGTGCCACCCTGAAGGAGTACCCGGCTGTGCGGTATCGGGGGGAA
TACAAGGACAATGCCCTGCTGGCTCAGCTAATCCAGGACAAGCTCGATGC
CTATAAAGCTGATGATCCAACAATGGGGGAGGGCCCAGACAAGGCACGCT
CCCAGCTCCTGATCCTGGATCGAGGCTTTGACCCCAGCTCCCCTGTGCTC
CATGAATTGACTTTTCAGGCTATGAGTTATGATCTGCTGCCTATCGAAAA
TGATGTATACAAGTATGAGACCAGCGGCATCGGGGAGGCACGGGTGAAGG
AGGTGCTCCTGGACGAGGACGACGACCTGTGGATAGCACTGCGCCACAAG
CACATCGCAGAGGTGTCCCAGGAAGTCACCCGGTCTCTGAAAGATTTTTC
TTCTAGCAAGAGAATGAATACTGGAGAGAAGACCACCATGCGGGACCTGT
CCCAGATGCTGAAGAAGATGCCTCAGTACCAGAAAGAGCTCAGCAAGTAC
TCCACCCACCTGCACCTTGCTGAGGACTGTATGAAGCATTACCAAGGCAC
CGTAGACAAACTCTGCCGAGTGGAGCAGGACCTGGCCATGGGCACAGATG
CTGAGGGAGAGAAGATCAAGGACCCTATGCGAGCCATCGTCCCCATTCTG
CTGGATGCCAATGTCAGCACTTATGACAAAATCCGCATCATCCTTCTCTA
CATCTTTTTGAAGAATGGCATCACGGAGGAAAACCTGAACAAACTGATCC
AGCACGCCCAGATACCCCCGGAGGATAGTGAGATCATCACCAACATGGCT
CACCTCGGCGTGCCCATCGTCACCGATTCCACGCTGCGTCGCCGGAGCAA
GCCGGAGCGGAAGGAACGCATCAGCGAGCAGACCTACCAGCTCTCACGGT
GGACTCCGATTATCAAGGACATCATGGAGGACACTATTGAGGACAAACTT
GACACCAAACACTACCCTTATATCTCTACCCGTTCCTCTGCCTCCTTCAG
CACCACCGCCGTCAGCGCCCGCTATGGGCACTGGCATAAGAACAAGGCCC
CAGGCGAGTACCGCAGTGGCCCCCGCCTCATCATTTTCATCCTTGGGGGT
GTGAGCCTGAATGAGATGCGCTGCGCCTACGAGGTGACCCAGGCCAACGG
AAAGTGGGAGGTGCTGATAGGATCCACACACATCCTCACCCCACAGAAAC
TGCTGGACACACTGAAGAAACTGAATAAAACAGATGAAGAAATAAGCAGT
TAAAAAAATAAGTCGCCCCTCCAAAACACGCCCCCATCCCACAGCGCTCC
GCAGCTTCCCACCACCGCCCGCCTCAGTTCCTTTGCGTCTGTTGCCTCCC
CAGCCCTGCACGCCCTGGCTGGCACTGTTGCCGCTGCATTCTCGTGTTCA
GTGATGCCCTCTTCTTGTTTGAAACAAAAGAAAATAATGCATTGTGTTTT
TTAAAAAGAGTATCTTATACATGTATCCTAAAAAGAGAAGCTCATGTGCA
ATTGGTGCACAGCAGGAGAAATTTCTGGACTGTTAGGATGAATGGACGCC
TTCTCCCCGTTATTTAAGATTTGTGACCTTGTACATAACCCTGGGTGACG
TGCACATTGCTTGGGTATGGAACGGTAGAAATTTGGGTGTTTTTAAAACC
TTGTTTGGGGTTGTTCCTGTCCTTGTTGAGAATCATAGAGATGTCTGTGT
TCTTGGAGTATTTCACACTGAGGACTAATCTGCTATCTTCATTCCAGTCC
CTACCCCTCAGTGCCTGCTCTCATCCAAATAACCTGGGAGGTGACAATCA
GGATATCTCAGGAGGTCCAAGGTGGAACAGACCTCTTTGCCTTTCCCAGC
GTCTCATACCCCCGGTAGTGCAGCTGTGGGTGGAGGCTGGGGTGTCTGCA
CGAAGTCAGGCCAGCGTCCTCCTCCACAGCCTGTCACTGCCCCCTCCCCA
GCCTGTGTCCACAGTGCTGTGATCCCGAGGGAAGTCCTCCAGTCTAAGTC
ACAGTGCCCTGACAGGTGAGAAGCAAACTCCCGCTGGAAGCCTCCATCTC
TTTGGAAAAACAGTTAGTCTGGAGCCTGTGGCCCAGGCCCTTCTGTCCCC
AGGCATCATCCCAACAGCTCATTTTCCCTAGTCCGCCTTCGTTCAAGGGT
CAGGAATGGACCAGAACAGATGGGTTCTGGAGGCCCCTGAACAGAGGGCT
ATGGCTGTGGAGAAGGTTCTTGGCCCGTTGGACTCACACAGACCCTGTAC
CCTCTCGGCAAGCATCTTCAGTCAGATTATCCTCAGTTTCAGATACTTCA
TAATACCTTGTGTTGTGTGGGGTCATACATCATCGTGTTTGTAAGAGAAG
ATGGTCATTTTATTCTCTGTATAAAACTTAGCTCTAAAGCAGAAACTAAA
GCAGCAAATGCAGGAAGGCTGTCTCGCCATCCTCAAGACTCAGCAGCTCT
CATTCTCCAGTGGTGAGCACACCATTTGTGCTGCTGCTGTTGTCGTGAAA
TATAATAACAGTGGAAGTCACAAAAATGTCCCCTGCCCAGCCCCCTCGCC
GCCCTTGACCTCCTGCAGGCCATGTGTGTATTACTTGTCTAGTGATGTCC
TCTCAAAGTGCTGTACGCGAGCTCGGCGCCACCTCCGCCTCCCTTTCAGA
GCCTGCTCCCCGCCCTCTCTGCTCGCTGCATTGTGGTGTTCTCTTCTCAA
GGCTTTGAAATCTCCCCTTGCACTGAGATTAGTCGTCAGATCTCTCCCCG
TCTCCCTCCCAACTTATACGACCTGATTTCCTTAGGACGGAACCGCAGGC
ACCTGCGCCGGGCGTCTTACTCCCGCTGCTTGTTCTGTCCCCTCCCTCGG
ACCAAACAGTGCTCATGCTTCAGGACCTTGTTTGTCGAAGATGTTGGTTT
CCCTTTCTCTGTTATTTATATAAAAATAATTTATCAAAAGGATATTTTAA
AAAAGCTAGTCTGTCTTGAAACTTGTTTACCTTAAAATTATCAGAATCTC
AGTGTTTGAAAGTACTGAAGCACAAACATATATCATCTCTGTACCATTCT
GTACTAAAGCACTTGAGTCTAATAAATAAAGAAATCAGCACCCCT

NM 001374307.2 Homo sapiens syntaxin binding protein 1 (STXBP1), transcript variant 4, mRNA (SEQ ID NO: 3)

AGTCGGTCCCTAGCGCGGCTGCGGGGCGGAGAGCTGCGGCTGGCCCAGCG
CGCCCACCTGAGGAGGCGGCGGGGTCCGCAGGCGTCGCGGGACGAGGAGA
TCGGAGCCGGGAGACTCGCGCAGCGCCATGGCCCCCATTGGCCTCAAAGC
TGTTGTCGGAGAGAGAAAGAGGAAGACTACCTAGACATGTTGCTCCAGAG
CAGAATTTCCAGCAGTAGATTGAACCTTCGGAGGCCGAGTCCCAGCCCAG
TGTAAACTGAGATTATGCATGATGTGATAAAGAAGGTCAAGAAGAAGGGG
GAATGGAAGGTGCTGGTGGTGGATCAGTTAAGCATGAGGATGCTGTCCTC
CTGCTGCAAGATGACAGACATCATGACCGAGGGCATAACGATTGTGGAAG
ATATCAATAAGCGCAGAGAGCCGCTCCCCAGCCTGGAGGCTGTGTATCTC
ATCACTCCATCCGAGAAGTCCGTCCACTCTCTCATCAGTGACTTTAAGGA
CCCGCCGACTGCTAAATACCGGGCTGCACACGTCTTCTTCACTGACTCTT
GTCCAGATGCCCTGTTTAATGAACTGGTAAAATCCCGAGCAGCCAAAGTC
ATCAAAACTCTGACGGAAATCAATATTGCATTTCTCCCGTATGAATCCCA
GGTCTATTCCTTGGACTCTGCTGACTCTTTCCAAAGCTTCTACAGTCCCC
ACAAGGCTCAGATGAAGAATCCTATACTGGAGCGCCTGGCAGAGCAGATC
GCGACCCTTTGTGCCACCCTGAAGGAGTACCCGGCTGTGCGGTATCGGGG
GGAATACAAGGACAATGCCCTGCTGGCTCAGCTAATCCAGGACAAGCTCG
ATGCCTATAAAGCTGATGATCCAACAATGGGGGAGGGCCCAGACAAGGCA
CGCTCCCAGCTCCTGATCCTGGATCGAGGCTTTGACCCCAGCTCCCCTGT
GCTCCATGAATTGACTTTTCAGGCTATGAGTTATGATCTGCTGCCTATCG
AAAATGATGTATACAAGTATGAGACCAGCGGCATCGGGGAGGCACGGGTG
AAGGAGGTGCTCCTGGACGAGGACGACGACCTGTGGATAGCACTGCGCCA
CAAGCACATCGCAGAGGTGTCCCAGGAAGTCACCCGGTCTCTGAAAGATT
TTTCTTCTAGCAAGAGAATGAATACTGGAGAGAAGACCACCATGCGGGAC
CTGTCCCAGATGCTGAAGAAGATGCCTCAGTACCAGAAAGAGCTCAGCAA
GTACTCCACCCACCTGCACCTTGCTGAGGACTGTATGAAGCATTACCAAG
GCACCGTAGACAAACTCTGCCGAGTGGAGCAGGACCTGGCCATGGGCACA
GATGCTGAGGGAGAGAAGATCAAGGACCCTATGCGAGCCATCGTCCCCAT
TCTGCTGGATGCCAATGTCAGCACTTATGACAAAATCCGCATCATCCTTC
TCTACATCTTTTTGAAGAATGGCATCACGGAGGAAAACCTGAACAAACTG
ATCCAGCACGCCCAGATACCCCCGGAGGATAGTGAGATCATCACCAACAT
GGCTCACCTCGGCGTGCCCATCGTCACCGATTCCACGCTGCGTCGCCGGA
GCAAGCCGGAGCGGAAGGAACGCATCAGCGAGCAGACCTACCAGCTCTCA
CGGTGGACTCCGATTATCAAGGACATCATGGAGGACACTATTGAGGACAA
ACTTGACACCAAACACTACCCTTATATCTCTACCCGTTCCTCTGCCTCCT
TCAGCACCACCGCCGTCAGCGCCCGCTATGGGCACTGGCATAAGAACAAG
GCCCCAGGCGAGTACCGCAGTGGCCCCCGCCTCATCATTTTCATCCTTGG
GGGTGTGAGCCTGAATGAGATGCGCTGCGCCTACGAGGTGACCCAGGCCA
ACGGAAAGTGGGAGGTGCTGATAGGTTCTACTCACATTCTTACTCCCACC
AAATTTCTCATGGACCTGAGACACCCCGACTTCAGGGAGTCCTCTAGGGT
ATCTTTTGAGGATCAGGCTCCAACAATGGAGTGAGAGCCAAAGAAACAAA
GATCCACACACATCCTCACCCCACAGAAACTGCTGGACACACTGAAGAAA
CTGAATAAAACAGATGAAGAAATAAGCAGTTAAAAAAATAAGTCGCCCCT
CCAAAACACGCCCCCATCCCACAGCGCTCCGCAGCTTCCCACCACCGCCC
GCCTCAGTTCCTTTGCGTCTGTTGCCTCCCCAGCCCTGCACGCCCTGGCT
GGCACTGTTGCCGCTGCATTCTCGTGTTCAGTGATGCCCTCTTCTTGTTT
GAAACAAAAGAAAATAATGCATTGTGTTTTTTAAAAAGAGTATCTTATAC
ATGTATCCTAAAAAGAGAAGCTCATGTGCAATTGGTGCACAGCAGGAGAA
ATTTCTGGACTGTTAGGATGAATGGACGCCTTCTCCCCGTTATTTAAGAT
TTGTGACCTTGTACATAACCCTGGGTGACGTGCACATTGCTTGGGTATGG
AACGGTAGAAATTTGGGTGTTTTTAAAACCTTGTTTGGGGTTGTTCCTGT
CCTTGTTGAGAATCATAGAGATGTCTGTGTTCTTGGAGTATTTCACACTG
AGGACTAATCTGCTATCTTCATTCCAGTCCCTACCCCTCAGTGCCTGCTC
TCATCCAAATAACCTGGGAGGTGACAATCAGGATATCTCAGGAGGTCCAA
GGTGGAACAGACCTCTTTGCCTTTCCCAGCGTCTCATACCCCCGGTAGTG
CAGCTGTGGGTGGAGGCTGGGGTGTCTGCACGAAGTCAGGCCAGCGTCCT
CCTCCACAGCCTGTCACTGCCCCCTCCCCAGCCTGTGTCCACAGTGCTGT
GATCCCGAGGGAAGTCCTCCAGTCTAAGTCACAGTGCCCTGACAGGTGAG
AAGCAAACTCCCGCTGGAAGCCTCCATCTCTTTGGAAAAACAGTTAGTCT
GGAGCCTGTGGCCCAGGCCCTTCTGTCCCCAGGCATCATCCCAACAGCTC
ATTTTCCCTAGTCCGCCTTCGTTCAAGGGTCAGGAATGGACCAGAACAGA
TGGGTTCTGGAGGCCCCTGAACAGAGGGCTATGGCTGTGGAGAAGGTTCT
TGGCCCGTTGGACTCACACAGACCCTGTACCCTCTCGGCAAGCATCTTCA
GTCAGATTATCCTCAGTTTCAGATACTTCATAATACCTTGTGTTGTGTGG
GGTCATACATCATCGTGTTTGTAAGAGAAGATGGTCATTTTATTCTCTGT
ATAAAACTTAGCTCTAAAGCAGAAACTAAAGCAGCAAATGCAGGAAGGCT
GTCTCGCCATCCTCAAGACTCAGCAGCTCTCATTCTCCAGTGGTGAGCAC
ACCATTTGTGCTGCTGCTGTTGTCGTGAAATATAATAACAGTGGAAGTCA
CAAAAATGTCCCCTGCCCAGCCCCCTCGCCGCCCTTGACCTCCTGCAGGC
CATGTGTGTATTACTTGTCTAGTGATGTCCTCTCAAAGTGCTGTACGCGA
GCTCGGCGCCACCTCCGCCTCCCTTTCAGAGCCTGCTCCCCGCCCTCTCT
GCTCGCTGCATTGTGGTGTTCTCTTCTCAAGGCTTTGAAATCTCCCCTTG
CACTGAGATTAGTCGTCAGATCTCTCCCCGTCTCCCTCCCAACTTATACG
ACCTGATTTCCTTAGGACGGAACCGCAGGCACCTGCGCCGGGCGTCTTAC
TCCCGCTGCTTGTTCTGTCCCCTCCCTCGGACCAAACAGTGCTCATGCTT
CAGGACCTTGTTTGTCGAAGATGTTGGTTTCCCTTTCTCTGTTATTTATA
TAAAAATAATTTATCAAAAGGATATTTTAAAAAAGCTAGTCTGTCTTGAA
ACTTGTTTACCTTAAAATTATCAGAATCTCAGTGTTTGAAAGTACTGAAG
CACAAACATATATCATCTCTGTACCATTCTGTACTAAAGCACTTGAGTCT
AATAAATAAAGAAATCAGCACCCCT

NM 001374308.2 Homo sapiens syntaxin binding protein 1 (STXBP1), transcript variant 5, mRNA (SEQ ID NO: 4)

AGTCGGTCCCTAGCGCGGCTGCGGGGCGGAGAGCTGCGGCTGGCCCAGCG
CGCCCACCTGAGGAGGCGGCGGGGTCCGCAGGCGTCGCGGGACGAGGAGA
TCGGAGCCGGGAGACTCGCGCAGCGCCATGGCCCCCATTGGCCTCAAAGC
TGTTGTCGGAGAGAGATATATACCTAGGAATGGAGTTGCTGGATCTCTAT
GTTTGGTTATTTGAGGAGCTGCCGGCCTGTTTTCCACATCAGCTGTACCA
TTTTACATTCTCACCAGCAGTGCATGACGGTCCAGTGCCTTTTTATCTTA
CTGCGCTTAAGGTGTAATTCCTTCTTTCTCAGACTGGACTGTAAATTCTT
TGAGAGATTATGCATGATGTGATAAAGAAGGTCAAGAAGAAGGGGGAATG
GAAGGTGCTGGTGGTGGATCAGTTAAGCATGAGGATGCTGTCCTCCTGCT
GCAAGATGACAGACATCATGACCGAGGGCATAACGATTGTGGAAGATATC
AATAAGCGCAGAGAGCCGCTCCCCAGCCTGGAGGCTGTGTATCTCATCAC
TCCATCCGAGAAGTCCGTCCACTCTCTCATCAGTGACTTTAAGGACCCGC
CGACTGCTAAATACCGGGCTGCACACGTCTTCTTCACTGACTCTTGTCCA
GATGCCCTGTTTAATGAACTGGTAAAATCCCGAGCAGCCAAAGTCATCAA
AACTCTGACGGAAATCAATATTGCATTTCTCCCGTATGAATCCCAGGTCT
ATTCCTTGGACTCTGCTGACTCTTTCCAAAGCTTCTACAGTCCCCACAAG
GCTCAGATGAAGAATCCTATACTGGAGCGCCTGGCAGAGCAGATCGCGAC
CCTTTGTGCCACCCTGAAGGAGTACCCGGCTGTGCGGTATCGGGGGGAAT
ACAAGGACAATGCCCTGCTGGCTCAGCTAATCCAGGACAAGCTCGATGCC
TATAAAGCTGATGATCCAACAATGGGGGAGGGCCCAGACAAGGCACGCTC
CCAGCTCCTGATCCTGGATCGAGGCTTTGACCCCAGCTCCCCTGTGCTCC
ATGAATTGACTTTTCAGGCTATGAGTTATGATCTGCTGCCTATCGAAAAT
GATGTATACAAGTATGAGACCAGCGGCATCGGGGAGGCACGGGTGAAGGA
GGTGCTCCTGGACGAGGACGACGACCTGTGGATAGCACTGCGCCACAAGC
ACATCGCAGAGGTGTCCCAGGAAGTCACCCGGTCTCTGAAAGATTTTTCT
TCTAGCAAGAGAATGAATACTGGAGAGAAGACCACCATGCGGGACCTGTC
CCAGATGCTGAAGAAGATGCCTCAGTACCAGAAAGAGCTCAGCAAGTACT
CCACCCACCTGCACCTTGCTGAGGACTGTATGAAGCATTACCAAGGCACC
GTAGACAAACTCTGCCGAGTGGAGCAGGACCTGGCCATGGGCACAGATGC
TGAGGGAGAGAAGATCAAGGACCCTATGCGAGCCATCGTCCCCATTCTGC
TGGATGCCAATGTCAGCACTTATGACAAAATCCGCATCATCCTTCTCTAC
ATCTTTTTGAAGAATGGCATCACGGAGGAAAACCTGAACAAACTGATCCA
GCACGCCCAGATACCCCCGGAGGATAGTGAGATCATCACCAACATGGCTC
ACCTCGGCGTGCCCATCGTCACCGATTCCACGCTGCGTCGCCGGAGCAAG
CCGGAGCGGAAGGAACGCATCAGCGAGCAGACCTACCAGCTCTCACGGTG
GACTCCGATTATCAAGGACATCATGGAGGACACTATTGAGGACAAACTTG
ACACCAAACACTACCCTTATATCTCTACCCGTTCCTCTGCCTCCTTCAGC
ACCACCGCCGTCAGCGCCCGCTATGGGCACTGGCATAAGAACAAGGCCCC
AGGCGAGTACCGCAGTGGCCCCCGCCTCATCATTTTCATCCTTGGGGGTG
TGAGCCTGAATGAGATGCGCTGCGCCTACGAGGTGACCCAGGCCAACGGA
AAGTGGGAGGTGCTGATAGGTTCTACTCACATTCTTACTCCCACCAAATT
TCTCATGGACCTGAGACACCCCGACTTCAGGGAGTCCTCTAGGGTATCTT
TTGAGGATCAGGCTCCAACAATGGAGTGAGAGCCAAAGAAACAAAGATCC
ACACACATCCTCACCCCACAGAAACTGCTGGACACACTGAAGAAACTGAA
TAAAACAGATGAAGAAATAAGCAGTTAAAAAAATAAGTCGCCCCTCCAAA
ACACGCCCCCATCCCACAGCGCTCCGCAGCTTCCCACCACCGCCCGCCTC
AGTTCCTTTGCGTCTGTTGCCTCCCCAGCCCTGCACGCCCTGGCTGGCAC
TGTTGCCGCTGCATTCTCGTGTTCAGTGATGCCCTCTTCTTGTTTGAAAC
AAAAGAAAATAATGCATTGTGTTTTTTAAAAAGAGTATCTTATACATGTA
TCCTAAAAAGAGAAGCTCATGTGCAATTGGTGCACAGCAGGAGAAATTTC
TGGACTGTTAGGATGAATGGACGCCTTCTCCCCGTTATTTAAGATTTGTG
ACCTTGTACATAACCCTGGGTGACGTGCACATTGCTTGGGTATGGAACGG
TAGAAATTTGGGTGTTTTTAAAACCTTGTTTGGGGTTGTTCCTGTCCTTG
TTGAGAATCATAGAGATGTCTGTGTTCTTGGAGTATTTCACACTGAGGAC
TAATCTGCTATCTTCATTCCAGTCCCTACCCCTCAGTGCCTGCTCTCATC
CAAATAACCTGGGAGGTGACAATCAGGATATCTCAGGAGGTCCAAGGTGG
AACAGACCTCTTTGCCTTTCCCAGCGTCTCATACCCCCGGTAGTGCAGCT
GTGGGTGGAGGCTGGGGTGTCTGCACGAAGTCAGGCCAGCGTCCTCCTCC
ACAGCCTGTCACTGCCCCCTCCCCAGCCTGTGTCCACAGTGCTGTGATCC
CGAGGGAAGTCCTCCAGTCTAAGTCACAGTGCCCTGACAGGTGAGAAGCA
AACTCCCGCTGGAAGCCTCCATCTCTTTGGAAAAACAGTTAGTCTGGAGC
CTGTGGCCCAGGCCCTTCTGTCCCCAGGCATCATCCCAACAGCTCATTTT
CCCTAGTCCGCCTTCGTTCAAGGGTCAGGAATGGACCAGAACAGATGGGT
TCTGGAGGCCCCTGAACAGAGGGCTATGGCTGTGGAGAAGGTTCTTGGCC
CGTTGGACTCACACAGACCCTGTACCCTCTCGGCAAGCATCTTCAGTCAG
ATTATCCTCAGTTTCAGATACTTCATAATACCTTGTGTTGTGTGGGGTCA
TACATCATCGTGTTTGTAAGAGAAGATGGTCATTTTATTCTCTGTATAAA
ACTTAGCTCTAAAGCAGAAACTAAAGCAGCAAATGCAGGAAGGCTGTCTC
GCCATCCTCAAGACTCAGCAGCTCTCATTCTCCAGTGGTGAGCACACCAT
TTGTGCTGCTGCTGTTGTCGTGAAATATAATAACAGTGGAAGTCACAAAA
ATGTCCCCTGCCCAGCCCCCTCGCCGCCCTTGACCTCCTGCAGGCCATGT
GTGTATTACTTGTCTAGTGATGTCCTCTCAAAGTGCTGTACGCGAGCTCG
GCGCCACCTCCGCCTCCCTTTCAGAGCCTGCTCCCCGCCCTCTCTGCTCG
CTGCATTGTGGTGTTCTCTTCTCAAGGCTTTGAAATCTCCCCTTGCACTG
AGATTAGTCGTCAGATCTCTCCCCGTCTCCCTCCCAACTTATACGACCTG
ATTTCCTTAGGACGGAACCGCAGGCACCTGCGCCGGGCGTCTTACTCCCG
CTGCTTGTTCTGTCCCCTCCCTCGGACCAAACAGTGCTCATGCTTCAGGA
CCTTGTTTGTCGAAGATGTTGGTTTCCCTTTCTCTGTTATTTATATAAAA
ATAATTTATCAAAAGGATATTTTAAAAAAGCTAGTCTGTCTTGAAACTTG
TTTACCTTAAAATTATCAGAATCTCAGTGTTTGAAAGTACTGAAGCACAA
ACATATATCATCTCTGTACCATTCTGTACTAAAGCACTTGAGTCTAATAA
ATAAAGAAATCAGCACCCCT

NM 001374309.2 Homo sapiens syntaxin binding protein 1 (STXBP1), transcript variant 6, mRNA (SEQ ID NO: 5)

AGAAAGAGGAAAGACAGGACCCGAGCGGGGTTTCAGGCAGATGGAGCGCG
TCGGTAGCCTGTGGCCAGGGATCCCAGCACCGACGGGAAAGAGGAGGCCT
GGGTACCCTGCGCCCCGGGCGCGCGCGGCGCGTGAGAGATTATGCATGAT
GTGATAAAGAAGGTCAAGAAGAAGGGGGAATGGAAGGTGCTGGTGGTGGA
TCAGTTAAGCATGAGGATGCTGTCCTCCTGCTGCAAGATGACAGACATCA
TGACCGAGGGCATAACGATTGTGGAAGATATCAATAAGCGCAGAGAGCCG
CTCCCCAGCCTGGAGGCTGTGTATCTCATCACTCCATCCGAGAAGTCCGT
CCACTCTCTCATCAGTGACTTTAAGGACCCGCCGACTGCTAAATACCGGG
CTGCACACGTCTTCTTCACTGACTCTTGTCCAGATGCCCTGTTTAATGAA
CTGGTAAAATCCCGAGCAGCCAAAGTCATCAAAACTCTGACGGAAATCAA
TATTGCATTTCTCCCGTATGAATCCCAGGTCTATTCCTTGGACTCTGCTG
ACTCTTTCCAAAGCTTCTACAGTCCCCACAAGGCTCAGATGAAGAATCCT
ATACTGGAGCGCCTGGCAGAGCAGATCGCGACCCTTTGTGCCACCCTGAA
GGAGTACCCGGCTGTGCGGTATCGGGGGGAATACAAGGACAATGCCCTGC
TGGCTCAGCTAATCCAGGACAAGCTCGATGCCTATAAAGCTGATGATCCA
ACAATGGGGGAGGGCCCAGACAAGGCACGCTCCCAGCTCCTGATCCTGGA
TCGAGGCTTTGACCCCAGCTCCCCTGTGCTCCATGAATTGACTTTTCAGG
CTATGAGTTATGATCTGCTGCCTATCGAAAATGATGTATACAAGTATGAG
ACCAGCGGCATCGGGGAGGCACGGGTGAAGGAGGTGCTCCTGGACGAGGA
CGACGACCTGTGGATAGCACTGCGCCACAAGCACATCGCAGAGGTGTCCC
AGGAAGTCACCCGGTCTCTGAAAGATTTTTCTTCTAGCAAGAGAATGAAT
ACTGGAGAGAAGACCACCATGCGGGACCTGTCCCAGATGCTGAAGAAGAT
GCCTCAGTACCAGAAAGAGCTCAGCAAGTACTCCACCCACCTGCACCTTG
CTGAGGACTGTATGAAGCATTACCAAGGCACCGTAGACAAACTCTGCCGA
GTGGAGCAGGACCTGGCCATGGGCACAGATGCTGAGGGAGAGAAGATCAA
GGACCCTATGCGAGCCATCGTCCCCATTCTGCTGGATGCCAATGTCAGCA
CTTATGACAAAATCCGCATCATCCTTCTCTACATCTTTTTGAAGAATGGC
ATCACGGAGGAAAACCTGAACAAACTGATCCAGCACGCCCAGATACCCCC
GGAGGATAGTGAGATCATCACCAACATGGCTCACCTCGGCGTGCCCATCG
TCACCGATTCCACGCTGCGTCGCCGGAGCAAGCCGGAGCGGAAGGAACGC
ATCAGCGAGCAGACCTACCAGCTCTCACGGTGGACTCCGATTATCAAGGA
CATCATGGAGGACACTATTGAGGACAAACTTGACACCAAACACTACCCTT
ATATCTCTACCCGTTCCTCTGCCTCCTTCAGCACCACCGCCGTCAGCGCC
CGCTATGGGCACTGGCATAAGAACAAGGCCCCAGGCGAGTACCGCAGTGG
CCCCCGCCTCATCATTTTCATCCTTGGGGGTGTGAGCCTGAATGAGATGC
GCTGCGCCTACGAGGTGACCCAGGCCAACGGAAAGTGGGAGGTGCTGATA
GGATCCACACACATCCTCACCCCACAGAAACTGCTGGACACACTGAAGAA
ACTGAATAAAACAGATGAAGAAATAAGCAGTTAAAAAAATAAGTCGCCCC
TCCAAAACACGCCCCCATCCCACAGCGCTCCGCAGCTTCCCACCACCGCC
CGCCTCAGTTCCTTTGCGTCTGTTGCCTCCCCAGCCCTGCACGCCCTGGC
TGGCACTGTTGCCGCTGCATTCTCGTGTTCAGTGATGCCCTCTTCTTGTT
TGAAACAAAAGAAAATAATGCATTGTGTTTTTTAAAAAGAGTATCTTATA
CATGTATCCTAAAAAGAGAAGCTCATGTGCAATTGGTGCACAGCAGGAGA
AATTTCTGGACTGTTAGGATGAATGGACGCCTTCTCCCCGTTATTTAAGA
TTTGTGACCTTGTACATAACCCTGGGTGACGTGCACATTGCTTGGGTATG
GAACGGTAGAAATTTGGGTGTTTTTAAAACCTTGTTTGGGGTTGTTCCTG
TCCTTGTTGAGAATCATAGAGATGTCTGTGTTCTTGGAGTATTTCACACT
GAGGACTAATCTGCTATCTTCATTCCAGTCCCTACCCCTCAGTGCCTGCT
CTCATCCAAATAACCTGGGAGGTGACAATCAGGATATCTCAGGAGGTCCA
AGGTGGAACAGACCTCTTTGCCTTTCCCAGCGTCTCATACCCCCGGTAGT
GCAGCTGTGGGTGGAGGCTGGGGTGTCTGCACGAAGTCAGGCCAGCGTCC
TCCTCCACAGCCTGTCACTGCCCCCTCCCCAGCCTGTGTCCACAGTGCTG
TGATCCCGAGGGAAGTCCTCCAGTCTAAGTCACAGTGCCCTGACAGGTGA
GAAGCAAACTCCCGCTGGAAGCCTCCATCTCTTTGGAAAAACAGTTAGTC
TGGAGCCTGTGGCCCAGGCCCTTCTGTCCCCAGGCATCATCCCAACAGCT
CATTTTCCCTAGTCCGCCTTCGTTCAAGGGTCAGGAATGGACCAGAACAG
ATGGGTTCTGGAGGCCCCTGAACAGAGGGCTATGGCTGTGGAGAAGGTTC
TTGGCCCGTTGGACTCACACAGACCCTGTACCCTCTCGGCAAGCATCTTC
AGTCAGATTATCCTCAGTTTCAGATACTTCATAATACCTTGTGTTGTGTG
GGGTCATACATCATCGTGTTTGTAAGAGAAGATGGTCATTTTATTCTCTG
TATAAAACTTAGCTCTAAAGCAGAAACTAAAGCAGCAAATGCAGGAAGGC
TGTCTCGCCATCCTCAAGACTCAGCAGCTCTCATTCTCCAGTGGTGAGCA
CACCATTTGTGCTGCTGCTGTTGTCGTGAAATATAATAACAGTGGAAGTC
ACAAAAATGTCCCCTGCCCAGCCCCCTCGCCGCCCTTGACCTCCTGCAGG
CCATGTGTGTATTACTTGTCTAGTGATGTCCTCTCAAAGTGCTGTACGCG
AGCTCGGCGCCACCTCCGCCTCCCTTTCAGAGCCTGCTCCCCGCCCTCTC
TGCTCGCTGCATTGTGGTGTTCTCTTCTCAAGGCTTTGAAATCTCCCCTT
GCACTGAGATTAGTCGTCAGATCTCTCCCCGTCTCCCTCCCAACTTATAC
GACCTGATTTCCTTAGGACGGAACCGCAGGCACCTGCGCCGGGCGTCTTA
CTCCCGCTGCTTGTTCTGTCCCCTCCCTCGGACCAAACAGTGCTCATGCT
TCAGGACCTTGTTTGTCGAAGATGTTGGTTTCCCTTTCTCTGTTATTTAT
ATAAAAATAATTTATCAAAAGGATATTTTAAAAAAGCTAGTCTGTCTTGA
AACTTGTTTACCTTAAAATTATCAGAATCTCAGTGTTTGAAAGTACTGAA
GCACAAACATATATCATCTCTGTACCATTCTGTACTAAAGCACTTGAGTC
TAATAAATAAAGAAATCAGCACCCCT

NM 001374310.2 Homo sapiens syntaxin binding protein 1 (STXBP1), transcript variant 7, mRNA (SEQ ID NO: 6)

AGTCGGTCCCTAGCGCGGCTGCGGGGCGGAGAGCTGCGGCTGGCCCAGCG
CGCCCACCTGAGGAGGCGGCGGGGTCCGCAGGCGTCGCGGGACGAGGAGA
TCGGAGCCGGGAGACTCGCGCAGCGCCATGGCCCCCATTGGCCTCAAAGC
TGTTGTCGGAGAGAGATATATACCTAGGAATGGAGTTGCTGGATCTCTAT
GTTTGGTTATTTGAGGAGCTGCCGGCCTGTTTTCCACATCAGCTGTACCA
TTTTACATTCTCACCAGCAGTGCATGACGGTCCAGTGCCTTTTTATCTTA
CTGCGCTTAAGGTGTAATTCCTTCTTTCTCAGACTGGACTGTAAATTCTT
TGAGAGATTATGCATGATGTGATAAAGAAGGTCAAGAAGAAGGGGGAATG
GAAGGTGCTGGTGGTGGATCAGTTAAGCATGAGGATGCTGTCCTCCTGCT
GCAAGATGACAGACATCATGACCGAGGGCATAACGATTGTGGAAGATATC
AATAAGCGCAGAGAGCCGCTCCCCAGCCTGGAGGCTGTGTATCTCATCAC
TCCATCCGAGAAGTCCGTCCACTCTCTCATCAGTGACTTTAAGGACCCGC
CGACTGCTAAATACCGGGCTGCACACGTCTTCTTCACTGACTCTTGTCCA
GATGCCCTGTTTAATGAACTGGTAAAATCCCGAGCAGCCAAAGTCATCAA
AACTCTGACGGAAATCAATATTGCATTTCTCCCGTATGAATCCCAGGTCT
ATTCCTTGGACTCTGCTGACTCTTTCCAAAGCTTCTACAGTCCCCACAAG
GCTCAGATGAAGAATCCTATACTGGAGCGCCTGGCAGAGCAGATCGCGAC
CCTTTGTGCCACCCTGAAGGAGTACCCGGCTGTGCGGTATCGGGGGGAAT
ACAAGGACAATGCCCTGCTGGCTCAGCTAATCCAGGACAAGCTCGATGCC
TATAAAGCTGATGATCCAACAATGGGGGAGGGCCCAGACAAGGCACGCTC
CCAGCTCCTGATCCTGGATCGAGGCTTTGACCCCAGCTCCCCTGTGCTCC
ATGAATTGACTTTTCAGGCTATGAGTTATGATCTGCTGCCTATCGAAAAT
GATGTATACAAGTATGAGACCAGCGGCATCGGGGAGGCACGGGTGAAGGA
GGTGCTCCTGGACGAGGACGACGACCTGTGGATAGCACTGCGCCACAAGC
ACATCGCAGAGGTGTCCCAGGAAGTCACCCGGTCTCTGAAAGATTTTTCT
TCTAGCAAGAGAATGAATACTGGAGAGAAGACCACCATGCGGGACCTGTC
CCAGATGCTGAAGAAGATGCCTCAGTACCAGAAAGAGCTCAGCAAGTACT
CCACCCACCTGCACCTTGCTGAGGACTGTATGAAGCATTACCAAGGCACC
GTAGACAAACTCTGCCGAGTGGAGCAGGACCTGGCCATGGGCACAGATGC
TGAGGGAGAGAAGATCAAGGACCCTATGCGAGCCATCGTCCCCATTCTGC
TGGATGCCAATGTCAGCACTTATGACAAAATCCGCATCATCCTTCTCTAC
ATCTTTTTGAAGAATGGCATCACGGAGGAAAACCTGAACAAACTGATCCA
GCACGCCCAGATACCCCCGGAGGATAGTGAGATCATCACCAACATGGCTC
ACCTCGGCGTGCCCATCGTCACCGATTCCACGCTGCGTCGCCGGAGCAAG
CCGGAGCGGAAGGAACGCATCAGCGAGCAGACCTACCAGCTCTCACGGTG
GACTCCGATTATCAAGGACATCATGGAGGACACTATTGAGGACAAACTTG
ACACCAAACACTACCCTTATATCTCTACCCGTTCCTCTGCCTCCTTCAGC
ACCACCGCCGTCAGCGCCCGCTATGGGCACTGGCATAAGAACAAGGCCCC
AGGCGAGTACCGCAGTGGCCCCCGCCTCATCATTTTCATCCTTGGGGGTG
TGAGCCTGAATGAGATGCGCTGCGCCTACGAGGTGACCCAGGCCAACGGA
AAGTGGGAGGTGCTGATAGGATCCACACACATCCTCACCCCACAGAAACT
GCTGGACACACTGAAGAAACTGAATAAAACAGATGAAGAAATAAGCAGTT
AAAAAAATAAGTCGCCCCTCCAAAACACGCCCCCATCCCACAGCGCTCCG
CAGCTTCCCACCACCGCCCGCCTCAGTTCCTTTGCGTCTGTTGCCTCCCC
AGCCCTGCACGCCCTGGCTGGCACTGTTGCCGCTGCATTCTCGTGTTCAG
TGATGCCCTCTTCTTGTTTGAAACAAAAGAAAATAATGCATTGTGTTTTT
TAAAAAGAGTATCTTATACATGTATCCTAAAAAGAGAAGCTCATGTGCAA
TTGGTGCACAGCAGGAGAAATTTCTGGACTGTTAGGATGAATGGACGCCT
TCTCCCCGTTATTTAAGATTTGTGACCTTGTACATAACCCTGGGTGACGT
GCACATTGCTTGGGTATGGAACGGTAGAAATTTGGGTGTTTTTAAAACCT
TGTTTGGGGTTGTTCCTGTCCTTGTTGAGAATCATAGAGATGTCTGTGTT
CTTGGAGTATTTCACACTGAGGACTAATCTGCTATCTTCATTCCAGTCCC
TACCCCTCAGTGCCTGCTCTCATCCAAATAACCTGGGAGGTGACAATCAG
GATATCTCAGGAGGTCCAAGGTGGAACAGACCTCTTTGCCTTTCCCAGCG
TCTCATACCCCCGGTAGTGCAGCTGTGGGTGGAGGCTGGGGTGTCTGCAC
GAAGTCAGGCCAGCGTCCTCCTCCACAGCCTGTCACTGCCCCCTCCCCAG
CCTGTGTCCACAGTGCTGTGATCCCGAGGGAAGTCCTCCAGTCTAAGTCA
CAGTGCCCTGACAGGTGAGAAGCAAACTCCCGCTGGAAGCCTCCATCTCT
TTGGAAAAACAGTTAGTCTGGAGCCTGTGGCCCAGGCCCTTCTGTCCCCA
GGCATCATCCCAACAGCTCATTTTCCCTAGTCCGCCTTCGTTCAAGGGTC
AGGAATGGACCAGAACAGATGGGTTCTGGAGGCCCCTGAACAGAGGGCTA
TGGCTGTGGAGAAGGTTCTTGGCCCGTTGGACTCACACAGACCCTGTACC
CTCTCGGCAAGCATCTTCAGTCAGATTATCCTCAGTTTCAGATACTTCAT
AATACCTTGTGTTGTGTGGGGTCATACATCATCGTGTTTGTAAGAGAAGA
TGGTCATTTTATTCTCTGTATAAAACTTAGCTCTAAAGCAGAAACTAAAG
CAGCAAATGCAGGAAGGCTGTCTCGCCATCCTCAAGACTCAGCAGCTCTC
ATTCTCCAGTGGTGAGCACACCATTTGTGCTGCTGCTGTTGTCGTGAAAT
ATAATAACAGTGGAAGTCACAAAAATGTCCCCTGCCCAGCCCCCTCGCCG
CCCTTGACCTCCTGCAGGCCATGTGTGTATTACTTGTCTAGTGATGTCCT
CTCAAAGTGCTGTACGCGAGCTCGGCGCCACCTCCGCCTCCCTTTCAGAG
CCTGCTCCCCGCCCTCTCTGCTCGCTGCATTGTGGTGTTCTCTTCTCAAG
GCTTTGAAATCTCCCCTTGCACTGAGATTAGTCGTCAGATCTCTCCCCGT
CTCCCTCCCAACTTATACGACCTGATTTCCTTAGGACGGAACCGCAGGCA
CCTGCGCCGGGCGTCTTACTCCCGCTGCTTGTTCTGTCCCCTCCCTCGGA
CCAAACAGTGCTCATGCTTCAGGACCTTGTTTGTCGAAGATGTTGGTTTC
CCTTTCTCTGTTATTTATATAAAAATAATTTATCAAAAGGATATTTTAAA
AAAGCTAGTCTGTCTTGAAACTTGTTTACCTTAAAATTATCAGAATCTCA
GTGTTTGAAAGTACTGAAGCACAAACATATATCATCTCTGTACCATTCTG
TACTAAAGCACTTGAGTCTAATAAATAAAGAAATCAGCACCCCT

NM 001374311.2 Homo sapiens syntaxin binding protein 1 (STXBP1), transcript variant 8, mRNA (SEQ ID NO: 7)

AGTCGGTCCCTAGCGCGGCTGCGGGGCGGAGAGCTGCGGCTGGCCCAGCG
CGCCCACCTGAGGAGGCGGCGGGGTCCGCAGGCGTCGCGGGACGAGGAGA
TCGGAGCCGGGAGACTCGCGCAGCGCCATGGCCCCCATTGGCCTCAAAGC
TGTTGTCGGAGAGAGAAAGAGGAAGACTACCTAGACATGTTGCTCCAGAG
CAGAATTTCCAGCAGTAGATTGAACCTTCGGAGGCCGAGTCCCAGCCCAG
TGTAAACTGAGATTATGCATGATGTGATAAAGAAGGTCAAGAAGAAGGGG
GAATGGAAGGTGCTGGTGGTGGATCAGTTAAGCATGAGGATGCTGTCCTC
CTGCTGCAAGATGACAGACATCATGACCGAGGGCATAACGATTGTGGAAG
ATATCAATAAGCGCAGAGAGCCGCTCCCCAGCCTGGAGGCTGTGTATCTC
ATCACTCCATCCGAGAAGTCCGTCCACTCTCTCATCAGTGACTTTAAGGA
CCCGCCGACTGCTAAATACCGGGCTGCACACGTCTTCTTCACTGACTCTT
GTCCAGATGCCCTGTTTAATGAACTGGTAAAATCCCGAGCAGCCAAAGTC
ATCAAAACTCTGACGGAAATCAATATTGCATTTCTCCCGTATGAATCCCA
GGTCTATTCCTTGGACTCTGCTGACTCTTTCCAAAGCTTCTACAGTCCCC
ACAAGGCTCAGATGAAGAATCCTATACTGGAGCGCCTGGCAGAGCAGATC
GCGACCCTTTGTGCCACCCTGAAGGAGTACCCGGCTGTGCGGTATCGGGG
GGAATACAAGGACAATGCCCTGCTGGCTCAGCTAATCCAGGACAAGCTCG
ATGCCTATAAAGCTGATGATCCAACAATGGGGGAGGGCCCAGACAAGGCA
CGCTCCCAGCTCCTGATCCTGGATCGAGGCTTTGACCCCAGCTCCCCTGT
GCTCCATGAATTGACTTTTCAGGCTATGAGTTATGATCTGCTGCCTATCG
AAAATGATGTATACAAGTATGAGACCAGCGGCATCGGGGAGGCACGGGTG
AAGGAGGTGCTCCTGGACGAGGACGACGACCTGTGGATAGCACTGCGCCA
CAAGCACATCGCAGAGGTGTCCCAGGAAGTCACCCGGTCTCTGAAAGATT
TTTCTTCTAGCAAGAGAATGAATACTGGAGAGAAGACCACCATGCGGGAC
CTGTCCCAGATGCTGAAGAAGATGCCTCAGTACCAGAAAGAGCTCAGCAA
GTACTCCACCCACCTGCACCTTGCTGAGGACTGTATGAAGCATTACCAAG
GCACCGTAGACAAACTCTGCCGAGTGGAGCAGGACCTGGCCATGGGCACA
GATGCTGAGGGAGAGAAGATCAAGGACCCTATGCGAGCCATCGTCCCCAT
TCTGCTGGATGCCAATGTCAGCACTTATGACAAAATCCGCATCATCCTTC
TCTACATCTTTTTGAAGAATGGCATCACGGAGGAAAACCTGAACAAACTG
ATCCAGCACGCCCAGATACCCCCGGAGGATAGTGAGATCATCACCAACAT
GGCTCACCTCGGCGTGCCCATCGTCACCGATTCCACGCTGCGTCGCCGGA
GCAAGCCGGAGCGGAAGGAACGCATCAGCGAGCAGACCTACCAGCTCTCA
CGGTGGACTCCGATTATCAAGGACATCATGGAGGACACTATTGAGGACAA
ACTTGACACCAAACACTACCCTTATATCTCTACCCGTTCCTCTGCCTCCT
TCAGCACCACCGCCGTCAGCGCCCGCTATGGGCACTGGCATAAGAACAAG
GCCCCAGGCGAGTACCGCAGTGGCCCCCGCCTCATCATTTTCATCCTTGG
GGGTGTGAGCCTGAATGAGATGCGCTGCGCCTACGAGGTGACCCAGGCCA
ACGGAAAGTGGGAGGTGCTGATAGGATCCACACACATCCTCACCCCACAG
AAACTGCTGGACACACTGAAGAAACTGAATAAAACAGATGAAGAAATAAG
CAGTTAAAAAAATAAGTCGCCCCTCCAAAACACGCCCCCATCCCACAGCG
CTCCGCAGCTTCCCACCACCGCCCGCCTCAGTTCCTTTGCGTCTGTTGCC
TCCCCAGCCCTGCACGCCCTGGCTGGCACTGTTGCCGCTGCATTCTCGTG
TTCAGTGATGCCCTCTTCTTGTTTGAAACAAAAGAAAATAATGCATTGTG
TTTTTTAAAAAGAGTATCTTATACATGTATCCTAAAAAGAGAAGCTCATG
TGCAATTGGTGCACAGCAGGAGAAATTTCTGGACTGTTAGGATGAATGGA
CGCCTTCTCCCCGTTATTTAAGATTTGTGACCTTGTACATAACCCTGGGT
GACGTGCACATTGCTTGGGTATGGAACGGTAGAAATTTGGGTGTTTTTAA
AACCTTGTTTGGGGTTGTTCCTGTCCTTGTTGAGAATCATAGAGATGTCT
GTGTTCTTGGAGTATTTCACACTGAGGACTAATCTGCTATCTTCATTCCA
GTCCCTACCCCTCAGTGCCTGCTCTCATCCAAATAACCTGGGAGGTGACA
ATCAGGATATCTCAGGAGGTCCAAGGTGGAACAGACCTCTTTGCCTTTCC
CAGCGTCTCATACCCCCGGTAGTGCAGCTGTGGGTGGAGGCTGGGGTGTC
TGCACGAAGTCAGGCCAGCGTCCTCCTCCACAGCCTGTCACTGCCCCCTC
CCCAGCCTGTGTCCACAGTGCTGTGATCCCGAGGGAAGTCCTCCAGTCTA
AGTCACAGTGCCCTGACAGGTGAGAAGCAAACTCCCGCTGGAAGCCTCCA
TCTCTTTGGAAAAACAGTTAGTCTGGAGCCTGTGGCCCAGGCCCTTCTGT
CCCCAGGCATCATCCCAACAGCTCATTTTCCCTAGTCCGCCTTCGTTCAA
GGGTCAGGAATGGACCAGAACAGATGGGTTCTGGAGGCCCCTGAACAGAG
GGCTATGGCTGTGGAGAAGGTTCTTGGCCCGTTGGACTCACACAGACCCT
GTACCCTCTCGGCAAGCATCTTCAGTCAGATTATCCTCAGTTTCAGATAC
TTCATAATACCTTGTGTTGTGTGGGGTCATACATCATCGTGTTTGTAAGA
GAAGATGGTCATTTTATTCTCTGTATAAAACTTAGCTCTAAAGCAGAAAC
TAAAGCAGCAAATGCAGGAAGGCTGTCTCGCCATCCTCAAGACTCAGCAG
CTCTCATTCTCCAGTGGTGAGCACACCATTTGTGCTGCTGCTGTTGTCGT
GAAATATAATAACAGTGGAAGTCACAAAAATGTCCCCTGCCCAGCCCCCT
CGCCGCCCTTGACCTCCTGCAGGCCATGTGTGTATTACTTGTCTAGTGAT
GTCCTCTCAAAGTGCTGTACGCGAGCTCGGCGCCACCTCCGCCTCCCTTT
CAGAGCCTGCTCCCCGCCCTCTCTGCTCGCTGCATTGTGGTGTTCTCTTC
TCAAGGCTTTGAAATCTCCCCTTGCACTGAGATTAGTCGTCAGATCTCTC
CCCGTCTCCCTCCCAACTTATACGACCTGATTTCCTTAGGACGGAACCGC
AGGCACCTGCGCCGGGCGTCTTACTCCCGCTGCTTGTTCTGTCCCCTCCC
TCGGACCAAACAGTGCTCATGCTTCAGGACCTTGTTTGTCGAAGATGTTG
GTTTCCCTTTCTCTGTTATTTATATAAAAATAATTTATCAAAAGGATATT
TTAAAAAAGCTAGTCTGTCTTGAAACTTGTTTACCTTAAAATTATCAGAA
TCTCAGTGTTTGAAAGTACTGAAGCACAAACATATATCATCTCTGTACCA
TTCTGTACTAAAGCACTTGAGTCTAATAAATAAAGAAATCAGCACCCCT

NM 001374312.2 Homo sapiens syntaxin binding protein 1 (STXBP1), transcript variant 9, mRNA (SEQ ID NO: 8)

AGTCGGTCCCTAGCGCGGCTGCGGGGCGGAGAGCTGCGGCTGGCCCAGCG
CGCCCACCTGAGGAGGCGGCGGGGTCCGCAGGCGTCGCGGGACGAGGAGA
TCGGAGCCGGGAGACTCGCGCAGCGCCATGGCCCCCATTGGCCTCAAAGC
TGTTGTCGGAGAGAGAAAGAGGAAGACTACCTAGACATGTTGCTCCAGAG
CAGAATTTCCAGCAGTAGATTGAACCTTCGGAGGCCGAGTCCCAGCCCAG
TGTAAACTGGTAAAGAAGATTATGCATGATGTGATAAAGAAGGTCAAGAA
GAAGGGGGAATGGAAGGTGCTGGTGGTGGATCAGTTAAGCATGAGGATGC
TGTCCTCCTGCTGCAAGATGACAGACATCATGACCGAGGGCATAACGATT
GTGGAAGATATCAATAAGCGCAGAGAGCCGCTCCCCAGCCTGGAGGCTGT
GTATCTCATCACTCCATCCGAGAAGTCCGTCCACTCTCTCATCAGTGACT
TTAAGGACCCGCCGACTGCTAAATACCGGGCTGCACACGTCTTCTTCACT
GACTCTTGTCCAGATGCCCTGTTTAATGAACTGGTAAAATCCCGAGCAGC
CAAAGTCATCAAAACTCTGACGGAAATCAATATTGCATTTCTCCCGTATG
AATCCCAGGTCTATTCCTTGGACTCTGCTGACTCTTTCCAAAGCTTCTAC
AGTCCCCACAAGGCTCAGATGAAGAATCCTATACTGGAGCGCCTGGCAGA
GCAGATCGCGACCCTTTGTGCCACCCTGAAGGAGTACCCGGCTGTGCGGT
ATCGGGGGGAATACAAGGACAATGCCCTGCTGGCTCAGCTAATCCAGGAC
AAGCTCGATGCCTATAAAGCTGATGATCCAACAATGGGGGAGGGCCCAGA
CAAGGCACGCTCCCAGCTCCTGATCCTGGATCGAGGCTTTGACCCCAGCT
CCCCTGTGCTCCATGAATTGACTTTTCAGGCTATGAGTTATGATCTGCTG
CCTATCGAAAATGATGTATACAAGTATGAGACCAGCGGCATCGGGGAGGC
ACGGGTGAAGGAGGTGCTCCTGGACGAGGACGACGACCTGTGGATAGCAC
TGCGCCACAAGCACATCGCAGAGGTGTCCCAGGAAGTCACCCGGTCTCTG
AAAGATTTTTCTTCTAGCAAGAGAATGAATACTGGAGAGAAGACCACCAT
GCGGGACCTGTCCCAGATGCTGAAGAAGATGCCTCAGTACCAGAAAGAGC
TCAGCAAGTACTCCACCCACCTGCACCTTGCTGAGGACTGTATGAAGCAT
TACCAAGGCACCGTAGACAAACTCTGCCGAGTGGAGCAGGACCTGGCCAT
GGGCACAGATGCTGAGGGAGAGAAGATCAAGGACCCTATGCGAGCCATCG
TCCCCATTCTGCTGGATGCCAATGTCAGCACTTATGACAAAATCCGCATC
ATCCTTCTCTACATCTTTTTGAAGAATGGCATCACGGAGGAAAACCTGAA
CAAACTGATCCAGCACGCCCAGATACCCCCGGAGGATAGTGAGATCATCA
CCAACATGGCTCACCTCGGCGTGCCCATCGTCACCGATTCCACGCTGCGT
CGCCGGAGCAAGCCGGAGCGGAAGGAACGCATCAGCGAGCAGACCTACCA
GCTCTCACGGTGGACTCCGATTATCAAGGACATCATGGAGGACACTATTG
AGGACAAACTTGACACCAAACACTACCCTTATATCTCTACCCGTTCCTCT
GCCTCCTTCAGCACCACCGCCGTCAGCGCCCGCTATGGGCACTGGCATAA
GAACAAGGCCCCAGGCGAGTACCGCAGTGGCCCCCGCCTCATCATTTTCA
TCCTTGGGGGTGTGAGCCTGAATGAGATGCGCTGCGCCTACGAGGTGACC
CAGGCCAACGGAAAGTGGGAGGTGCTGATAGGATCCACACACATCCTCAC
CCCACAGAAACTGCTGGACACACTGAAGAAACTGAATAAAACAGATGAAG
AAATAAGCAGTTAAAAAAATAAGTCGCCCCTCCAAAACACGCCCCCATCC
CACAGCGCTCCGCAGCTTCCCACCACCGCCCGCCTCAGTTCCTTTGCGTC
TGTTGCCTCCCCAGCCCTGCACGCCCTGGCTGGCACTGTTGCCGCTGCAT
TCTCGTGTTCAGTGATGCCCTCTTCTTGTTTGAAACAAAAGAAAATAATG
CATTGTGTTTTTTAAAAAGAGTATCTTATACATGTATCCTAAAAAGAGAA
GCTCATGTGCAATTGGTGCACAGCAGGAGAAATTTCTGGACTGTTAGGAT
GAATGGACGCCTTCTCCCCGTTATTTAAGATTTGTGACCTTGTACATAAC
CCTGGGTGACGTGCACATTGCTTGGGTATGGAACGGTAGAAATTTGGGTG
TTTTTAAAACCTTGTTTGGGGTTGTTCCTGTCCTTGTTGAGAATCATAGA
GATGTCTGTGTTCTTGGAGTATTTCACACTGAGGACTAATCTGCTATCTT
CATTCCAGTCCCTACCCCTCAGTGCCTGCTCTCATCCAAATAACCTGGGA
GGTGACAATCAGGATATCTCAGGAGGTCCAAGGTGGAACAGACCTCTTTG
CCTTTCCCAGCGTCTCATACCCCCGGTAGTGCAGCTGTGGGTGGAGGCTG
GGGTGTCTGCACGAAGTCAGGCCAGCGTCCTCCTCCACAGCCTGTCACTG
CCCCCTCCCCAGCCTGTGTCCACAGTGCTGTGATCCCGAGGGAAGTCCTC
CAGTCTAAGTCACAGTGCCCTGACAGGTGAGAAGCAAACTCCCGCTGGAA
GCCTCCATCTCTTTGGAAAAACAGTTAGTCTGGAGCCTGTGGCCCAGGCC
CTTCTGTCCCCAGGCATCATCCCAACAGCTCATTTTCCCTAGTCCGCCTT
CGTTCAAGGGTCAGGAATGGACCAGAACAGATGGGTTCTGGAGGCCCCTG
AACAGAGGGCTATGGCTGTGGAGAAGGTTCTTGGCCCGTTGGACTCACAC
AGACCCTGTACCCTCTCGGCAAGCATCTTCAGTCAGATTATCCTCAGTTT
CAGATACTTCATAATACCTTGTGTTGTGTGGGGTCATACATCATCGTGTT
TGTAAGAGAAGATGGTCATTTTATTCTCTGTATAAAACTTAGCTCTAAAG
CAGAAACTAAAGCAGCAAATGCAGGAAGGCTGTCTCGCCATCCTCAAGAC
TCAGCAGCTCTCATTCTCCAGTGGTGAGCACACCATTTGTGCTGCTGCTG
TTGTCGTGAAATATAATAACAGTGGAAGTCACAAAAATGTCCCCTGCCCA
GCCCCCTCGCCGCCCTTGACCTCCTGCAGGCCATGTGTGTATTACTTGTC
TAGTGATGTCCTCTCAAAGTGCTGTACGCGAGCTCGGCGCCACCTCCGCC
TCCCTTTCAGAGCCTGCTCCCCGCCCTCTCTGCTCGCTGCATTGTGGTGT
TCTCTTCTCAAGGCTTTGAAATCTCCCCTTGCACTGAGATTAGTCGTCAG
ATCTCTCCCCGTCTCCCTCCCAACTTATACGACCTGATTTCCTTAGGACG
GAACCGCAGGCACCTGCGCCGGGCGTCTTACTCCCGCTGCTTGTTCTGTC
CCCTCCCTCGGACCAAACAGTGCTCATGCTTCAGGACCTTGTTTGTCGAA
GATGTTGGTTTCCCTTTCTCTGTTATTTATATAAAAATAATTTATCAAAA
GGATATTTTAAAAAAGCTAGTCTGTCTTGAAACTTGTTTACCTTAAAATT
ATCAGAATCTCAGTGTTTGAAAGTACTGAAGCACAAACATATATCATCTC
TGTACCATTCTGTACTAAAGCACTTGAGTCTAATAAATAAAGAAATCAGC
ACCCCT

NM 001374313.2 Homo sapiens syntaxin binding protein 1 (STXBP1), transcript variant 10, mRNA (SEQ ID NO: 9)

AGTCGGTCCCTAGCGCGGCTGCGGGGCGGAGAGCTGCGGCTGGCCCAGCG
CGCCCACCTGAGGAGGCGGCGGGGTCCGCAGGCGTCGCGGGACGAGGAGA
TCGGAGCCGGGAGACTCGCGCAGCGCCATGGCCCCCATTGGCCTCAAAGC
TGTTGTCGGAGAGAAGATTATGCATGATGTGATAAAGAAGGTCAAGAAGA
AGGGGGAATGGAAGGTGCTGGTGGTGGATCAGTTAAGCATGAGGATGCTG
TCCTCCTGCTGCAAGATGACAGACATCATGACCGAGGGCATAACGATTGT
GGAAGATATCAATAAGCGCAGAGAGCCGCTCCCCAGCCTGGAGGCTGTGT
ATCTCATCACTCCATCCGAGAAGTCCGTCCACTCTCTCATCAGTGACTTT
AAGGACCCGCCGACTGCTAAATACCGGGCTGCACACGTCTTCTTCACTGA
CTCTTGTCCAGATGCCCTGTTTAATGAACTGGTAAAATCCCGAGCAGCCA
AAGTCATCAAAACTCTGACGGAAATCAATATTGCATTTCTCCCGTATGAA
TCCCAGGTCTATTCCTTGGACTCTGCTGACTCTTTCCAAAGCTTCTACAG
TCCCCACAAGGCTCAGATGAAGAATCCTATACTGGAGCGCCTGGCAGAGC
AGATCGCGACCCTTTGTGCCACCCTGAAGGAGTACCCGGCTGTGCGGTAT
CGGGGGGAATACAAGGACAATGCCCTGCTGGCTCAGCTAATCCAGGACAA
GCTCGATGCCTATAAAGCTGATGATCCAACAATGGGGGAGGGCCCAGACA
AGGCACGCTCCCAGCTCCTGATCCTGGATCGAGGCTTTGACCCCAGCTCC
CCTGTGCTCCATGAATTGACTTTTCAGGCTATGAGTTATGATCTGCTGCC
TATCGAAAATGATGTATACAAGTATGAGACCAGCGGCATCGGGGAGGCAC
GGGTGAAGGAGGTGCTCCTGGACGAGGACGACGACCTGTGGATAGCACTG
CGCCACAAGCACATCGCAGAGGTGTCCCAGGAAGTCACCCGGTCTCTGAA
AGATTTTTCTTCTAGCAAGAGAATGAATACTGGAGAGAAGACCACCATGC
GGGACCTGTCCCAGATGCTGAAGAAGATGCCTCAGTACCAGAAAGAGCTC
AGCAAGTACTCCACCCACCTGCACCTTGCTGAGGACTGTATGAAGCATTA
CCAAGGCACCGTAGACAAACTCTGCCGAGTGGAGCAGGACCTGGCCATGG
GCACAGATGCTGAGGGAGAGAAGATCAAGGACCCTATGCGAGCCATCGTC
CCCATTCTGCTGGATGCCAATGTCAGCACTTATGACAAAATCCGCATCAT
CCTTCTCTACATCTTTTTGAAGAATGGCATCACGGAGGAAAACCTGAACA
AACTGATCCAGCACGCCCAGATACCCCCGGAGGATAGTGAGATCATCACC
AACATGGCTCACCTCGGCGTGCCCATCGTCACCGATTCCACGCTGCGTCG
CCGGAGCAAGCCGGAGCGGAAGGAACGCATCAGCGAGCAGACCTACCAGC
TCTCACGGTGGACTCCGATTATCAAGGACATCATGGAGGACACTATTGAG
GACAAACTTGACACCAAACACTACCCTTATATCTCTACCCGTTCCTCTGC
CTCCTTCAGCACCACCGCCGTCAGCGCCCGCTATGGGCACTGGCATAAGA
ACAAGGCCCCAGGCGAGTACCGCAGTGGCCCCCGCCTCATCATTTTCATC
CTTGGGGGTGTGAGCCTGAATGAGATGCGCTGCGCCTACGAGGTGACCCA
GGCCAACGGAAAGTGGGAGGTGCTGATAGGGGAATGGCCCTTCAGAACCT
TGGCTAGGTGACATCAGAAGCTGCTTGTGTATGTAAGGAAAATGGGGCTT
CCTCCTAAGGATCCACACACATCCTCACCCCACAGAAACTGCTGGACACA
CTGAAGAAACTGAATAAAACAGATGAAGAAATAAGCAGTTAAAAAAATAA
GTCGCCCCTCCAAAACACGCCCCCATCCCACAGCGCTCCGCAGCTTCCCA
CCACCGCCCGCCTCAGTTCCTTTGCGTCTGTTGCCTCCCCAGCCCTGCAC
GCCCTGGCTGGCACTGTTGCCGCTGCATTCTCGTGTTCAGTGATGCCCTC
TTCTTGTTTGAAACAAAAGAAAATAATGCATTGTGTTTTTTAAAAAGAGT
ATCTTATACATGTATCCTAAAAAGAGAAGCTCATGTGCAATTGGTGCACA
GCAGGAGAAATTTCTGGACTGTTAGGATGAATGGACGCCTTCTCCCCGTT
ATTTAAGATTTGTGACCTTGTACATAACCCTGGGTGACGTGCACATTGCT
TGGGTATGGAACGGTAGAAATTTGGGTGTTTTTAAAACCTTGTTTGGGGT
TGTTCCTGTCCTTGTTGAGAATCATAGAGATGTCTGTGTTCTTGGAGTAT
TTCACACTGAGGACTAATCTGCTATCTTCATTCCAGTCCCTACCCCTCAG
TGCCTGCTCTCATCCAAATAACCTGGGAGGTGACAATCAGGATATCTCAG
GAGGTCCAAGGTGGAACAGACCTCTTTGCCTTTCCCAGCGTCTCATACCC
CCGGTAGTGCAGCTGTGGGTGGAGGCTGGGGTGTCTGCACGAAGTCAGGC
CAGCGTCCTCCTCCACAGCCTGTCACTGCCCCCTCCCCAGCCTGTGTCCA
CAGTGCTGTGATCCCGAGGGAAGTCCTCCAGTCTAAGTCACAGTGCCCTG
ACAGGTGAGAAGCAAACTCCCGCTGGAAGCCTCCATCTCTTTGGAAAAAC
AGTTAGTCTGGAGCCTGTGGCCCAGGCCCTTCTGTCCCCAGGCATCATCC
CAACAGCTCATTTTCCCTAGTCCGCCTTCGTTCAAGGGTCAGGAATGGAC
CAGAACAGATGGGTTCTGGAGGCCCCTGAACAGAGGGCTATGGCTGTGGA
GAAGGTTCTTGGCCCGTTGGACTCACACAGACCCTGTACCCTCTCGGCAA
GCATCTTCAGTCAGATTATCCTCAGTTTCAGATACTTCATAATACCTTGT
GTTGTGTGGGGTCATACATCATCGTGTTTGTAAGAGAAGATGGTCATTTT
ATTCTCTGTATAAAACTTAGCTCTAAAGCAGAAACTAAAGCAGCAAATGC
AGGAAGGCTGTCTCGCCATCCTCAAGACTCAGCAGCTCTCATTCTCCAGT
GGTGAGCACACCATTTGTGCTGCTGCTGTTGTCGTGAAATATAATAACAG
TGGAAGTCACAAAAATGTCCCCTGCCCAGCCCCCTCGCCGCCCTTGACCT
CCTGCAGGCCATGTGTGTATTACTTGTCTAGTGATGTCCTCTCAAAGTGC
TGTACGCGAGCTCGGCGCCACCTCCGCCTCCCTTTCAGAGCCTGCTCCCC
GCCCTCTCTGCTCGCTGCATTGTGGTGTTCTCTTCTCAAGGCTTTGAAAT
CTCCCCTTGCACTGAGATTAGTCGTCAGATCTCTCCCCGTCTCCCTCCCA
ACTTATACGACCTGATTTCCTTAGGACGGAACCGCAGGCACCTGCGCCGG
GCGTCTTACTCCCGCTGCTTGTTCTGTCCCCTCCCTCGGACCAAACAGTG
CTCATGCTTCAGGACCTTGTTTGTCGAAGATGTTGGTTTCCCTTTCTCTG
TTATTTATATAAAAATAATTTATCAAAAGGATATTTTAAAAAAGCTAGTC
TGTCTTGAAACTTGTTTACCTTAAAATTATCAGAATCTCAGTGTTTGAAA
GTACTGAAGCACAAACATATATCATCTCTGTACCATTCTGTACTAAAGCA
CTTGAGTCTAATAAATAAAGAAATCAGCACCCCT

NM_001374314.1 Homo sapiens syntaxin binding protein 1 (STXBP1), transcript variant 11, mRNA (SEQ ID NO: 10)

AGTCGGTCCCTAGCGCGGCTGCGGGGCGGAGAGCTGCGGCTGGCCCAGCG
CGCCCACCTGAGGAGGCGGCGGGGTCCGCAGGCGTCGCGGGACGAGGAGA
TCGGAGCCGGGAGACTCGCGCAGCGCCATGGCCCCCATTGGCCTCAAAGC
TGTTGTCGGAGAGAAGATTATGCATGATGTGATAAAGAAGGTCAAGAAGA
AGGGGGAATGGAAGGTGCTGGTGGTGGATCAGTTAAGCATGAGGATGCTG
TCCTCCTGCTGCAAGATGACAGACATCATGACCGAGGGCATAACGATTGT
GGAAGATATCAATAAGCGCAGAGAGCCGCTCCCCAGCCTGGAGGCTGTGT
ATCTCATCACTCCATCCGAGAAGTCCGTCCACTCTCTCATCAGTGACTTT
AAGGACCCGCCGACTGCTAAATACCGGGCTGCACACGTCTTCTTCACTGA
CTCTTGTCCAGATGCCCTGTTTAATGAACTGGTAAAATCCCGAGCAGCCA
AAGTCATCAAAACTCTGACGGAAATCAATATTGCATTTCTCCCGTATGAA
TCCCAGGTCTATTCCTTGGACTCTGCTGACTCTTTCCAAAGCTTCTACAG
TCCCCACAAGGCTCAGATGAAGAATCCTATACTGGAGCGCCTGGCAGAGC
AGATCGCGACCCTTTGTGCCACCCTGAAGGAGTACCCGGCTGTGCGGTAT
CGGGGGGAATACAAGGACAATGCCCTGCTGGCTCAGCTAATCCAGGACAA
GCTCGATGCCTATAAAGCTGATGATCCAACAATGGGGGAGGGCCCAGACA
AGGCACGCTCCCAGCTCCTGATCCTGGATCGAGGCTTTGACCCCAGCTCC
CCTGTGCTCCATGAATTGACTTTTCAGGCTATGAGTTATGATCTGCTGCC
TATCGAAAATGATGTATACAAGTATGAGACCAGCGGCATCGGGGAGGCAC
GGGTGAAGGAGGTGCTCCTGGACGAGGACGACGACCTGTGGATAGCACTG
CGCCACAAGCACATCGCAGAGGTGTCCCAGGAAGTCACCCGGTCTCTGAA
AGATTTTTCTTCTAGCAAGAGAATGAATACTGGAGAGAAGACCACCATGC
GGGACCTGTCCCAGATGCTGAAGAAGATGCCTCAGTACCAGAAAGAGCTC
AGCAAGTACTCCACCCACCTGCACCTTGCTGAGGACTGTATGAAGCATTA
CCAAGGCACCGTAGACAAACTCTGCCGAGTGGAGCAGGACCTGGCCATGG
GCACAGATGCTGAGGGAGAGAAGATCAAGGACCCTATGCGAGCCATCGTC
CCCATTCTGCTGGATGCCAATGTCAGCACTTATGACAAAATCCGCATCAT
CCTTCTCTACATCTTTTTGAAGAATGGCATCACGGAGGAAAACCTGAACA
AACTGATCCAGCACGCCCAGATACCCCCGGAGGATAGTGAGATCATCACC
AACATGGCTCACCTCGGCGTGCCCATCGTCACCGATTCCACGCTGCGTCG
CCGGAGCAAGCCGGAGCGGAAGGAACGCATCAGCGAGCAGACCTACCAGC
TCTCACGGTGGACTCCGATTATCAAGGACATCATGGAGGACACTATTGAG
GACAAACTTGACACCAAACACTACCCTTATATCTCTACCCGTTCCTCTGC
CTCCTTCAGCACCACCGCCGTCAGCGCCCGCTATGGGCACTGGCATAAGA
ACAAGGCCCCAGGCGAGTACCGCAGTGGCCCCCGCCTCATCATTTTCATC
CTTGGGGGTGTGAGCCTGAATGAGATGCGCTGCGCCTACGAGGTGACCCA
GGCCAACGGAAAGTGGGAGGTGCTGATAGTGCCCGTGGAGGCGGGAAGCT
GAGCCCGGCTCCCAGGAAGCACAGTGAGGGCTCAGCCATTGATGATGATG
ATGATGATGATGATGATGATGATGATGATGGTGATGATGATGATGCTGCT
GCTGTTGACGATGAGTCCAAGCCAAGCCTGGGGAGAAAAGAATAAATAGT
CTCATAAATTTAAAAAAGCCCAATGCTCATCAAGAAGAGAACAGATAAAC
TGTGTATTCACCCAATGGTGAGTTTAAAGCAGTTAAAAATGCATGACACA
CAGTTGCATATGTCAACATGAATAGATCTCAGACACTTAACAATGAGTGA
AAAGAGCACATTATGGGAGGACACAGACAGGATAGTGCATTTATATAAAG
CTTCAATGTTGCAGGCTGAAAGAGTGAGGGTCGTGATCAACTCAGTATCC
TGGAGGCTACATGGGTAAACAGCAAACTGTTCTCATAAATGCAGAATGTT
GGCAAACTGACAAACTGCATCTGCCGCCCAGAAGGAATGCGGAGGGCAGC
CACTCCCTAAGCGCAGTTTTCTTGTGATTAGGTACATCTGAAGCCTGTTA
GCAATAATGTGAACCTGTGATCAATTAAGCAGCTGACCAATCATTACCTC
CTCTTCCCTGCTCTTTCTACCCAGTAAATACAAAGGGCTGTAGAAGCTCA
GAGCTGCTGCTTTTGCTCAGTAGAAGCAGGGAGTCCTCTTCTTCTTCCCC
CGACCCCTTCTTTTAAAACAGTTTCTTTTAAGTTTTCATTTCTGCGTTCA
TCCTCCTTCATTCAGTCCCGTAGTAACCGTGGCAAACCACGGCACTTCAA
AACCTCATAAAAAGCACCACATATTGCTTATTTGGATACATATGTTGTAA
ATGTGTGAAAACATGCATGGGAATGACAAATACCAAAGTCAGGATAGTGA
CTGGCCATGGGGAAGGGATGGTTATTATTGTCGTCTTATATCTTCAATTC
CAAATGAGCCACGGGCTTAAAAGTACTTCATTGAATAAAAAAAAACCAAC
AATGTATTAATGCAA

NM_001374315.2 Homo sapiens syntaxin binding protein 1 (STXBP1), transcript variant 12, mRNA (SEQ ID NO: 11)

AGTCGGTCCCTAGCGCGGCTGCGGGGCGGAGAGCTGCGGCTGGCCCAGCG
CGCCCACCTGAGGAGGCGGCGGGGTCCGCAGGCGTCGCGGGACGAGGAGA
TCGGAGCCGGGAGACTCGCGCAGCGCCATGGCCCCCATTGGCCTCAAAGC
TGTTGTCGGAGAGAAGATTATGCATGATGTGATAAAGAAGGTCAAGAAGA
AGGGGGAATGGAAGGTGCTGGTGGTGGATCAGTTAAGCATGAGGATGCTG
TCCTCCTGCTGCAAGATGACAGACATCATGACCGAGGGCATAACGATTGT
GGAAGATATCAATAAGCGCAGAGAGCCGCTCCCCAGCCTGGAGGCTGTGT
ATCTCATCACTCCATCCGAGAAGTCCGTCCACTCTCTCATCAGTGACTTT
AAGGACCCGCCGACTGCTAAATACCGGGCTGCACACGTCTTCTTCACTGA
CTCTTGTCCAGATGCCCTGTTTAATGAACTGGTAAAATCCCGAGCAGCCA
AAGTCATCAAAACTCTGACGGAAATCAATATTGCATTTCTCCCGTATGAA
TCCCAGGTCTATTCCTTGGACTCTGCTGACTCTTTCCAAAGCTTCTACAG
TCCCCACAAGGCTCAGATGAAGAATCCTATACTGGAGCGCCTGGCAGAGC
AGATCGCGACCCTTTGTGCCACCCTGAAGGAGTACCCGGCTGTGCGGTAT
CGGGGGGAATACAAGGACAATGCCCTGCTGGCTCAGCTAATCCAGGACAA
GCTCGATGCCTATAAAGCTGATGATCCAACAATGGGGGAGGGCCCAGACA
AGGCACGCTCCCAGCTCCTGATCCTGGATCGAGGCTTTGACCCCAGCTCC
CCTGTGCTCCATGAATTGACTTTTCAGGCTATGAGTTATGATCTGCTGCC
TATCGAAAATGATGTATACAAGGAAGTCACCCGGTCTCTGAAAGATTTTT
CTTCTAGCAAGAGAATGAATACTGGAGAGAAGACCACCATGCGGGACCTG
TCCCAGATGCTGAAGAAGATGCCTCAGTACCAGAAAGAGCTCAGCAAGTA
CTCCACCCACCTGCACCTTGCTGAGGACTGTATGAAGCATTACCAAGGCA
CCGTAGACAAACTCTGCCGAGTGGAGCAGGACCTGGCCATGGGCACAGAT
GCTGAGGGAGAGAAGATCAAGGACCCTATGCGAGCCATCGTCCCCATTCT
GCTGGATGCCAATGTCAGCACTTATGACAAAATCCGCATCATCCTTCTCT
ACATCTTTTTGAAGAATGGCATCACGGAGGAAAACCTGAACAAACTGATC
CAGCACGCCCAGATACCCCCGGAGGATAGTGAGATCATCACCAACATGGC
TCACCTCGGCGTGCCCATCGTCACCGATTCCACGCTGCGTCGCCGGAGCA
AGCCGGAGCGGAAGGAACGCATCAGCGAGCAGACCTACCAGCTCTCACGG
TGGACTCCGATTATCAAGGACATCATGGAGGACACTATTGAGGACAAACT
TGACACCAAACACTACCCTTATATCTCTACCCGTTCCTCTGCCTCCTTCA
GCACCACCGCCGTCAGCGCCCGCTATGGGCACTGGCATAAGAACAAGGCC
CCAGGCGAGTACCGCAGTGGCCCCCGCCTCATCATTTTCATCCTTGGGGG
TGTGAGCCTGAATGAGATGCGCTGCGCCTACGAGGTGACCCAGGCCAACG
GAAAGTGGGAGGTGCTGATAGGTTCTACTCACATTCTTACTCCCACCAAA
TTTCTCATGGACCTGAGACACCCCGACTTCAGGGAGTCCTCTAGGGTATC
TTTTGAGGATCAGGCTCCAACAATGGAGTGAGAGCCAAAGAAACAAAGAT
CCACACACATCCTCACCCCACAGAAACTGCTGGACACACTGAAGAAACTG
AATAAAACAGATGAAGAAATAAGCAGTTAAAAAAATAAGTCGCCCCTCCA
AAACACGCCCCCATCCCACAGCGCTCCGCAGCTTCCCACCACCGCCCGCC
TCAGTTCCTTTGCGTCTGTTGCCTCCCCAGCCCTGCACGCCCTGGCTGGC
ACTGTTGCCGCTGCATTCTCGTGTTCAGTGATGCCCTCTTCTTGTTTGAA
ACAAAAGAAAATAATGCATTGTGTTTTTTAAAAAGAGTATCTTATACATG
TATCCTAAAAAGAGAAGCTCATGTGCAATTGGTGCACAGCAGGAGAAATT
TCTGGACTGTTAGGATGAATGGACGCCTTCTCCCCGTTATTTAAGATTTG
TGACCTTGTACATAACCCTGGGTGACGTGCACATTGCTTGGGTATGGAAC
GGTAGAAATTTGGGTGTTTTTAAAACCTTGTTTGGGGTTGTTCCTGTCCT
TGTTGAGAATCATAGAGATGTCTGTGTTCTTGGAGTATTTCACACTGAGG
ACTAATCTGCTATCTTCATTCCAGTCCCTACCCCTCAGTGCCTGCTCTCA
TCCAAATAACCTGGGAGGTGACAATCAGGATATCTCAGGAGGTCCAAGGT
GGAACAGACCTCTTTGCCTTTCCCAGCGTCTCATACCCCCGGTAGTGCAG
CTGTGGGTGGAGGCTGGGGTGTCTGCACGAAGTCAGGCCAGCGTCCTCCT
CCACAGCCTGTCACTGCCCCCTCCCCAGCCTGTGTCCACAGTGCTGTGAT
CCCGAGGGAAGTCCTCCAGTCTAAGTCACAGTGCCCTGACAGGTGAGAAG
CAAACTCCCGCTGGAAGCCTCCATCTCTTTGGAAAAACAGTTAGTCTGGA
GCCTGTGGCCCAGGCCCTTCTGTCCCCAGGCATCATCCCAACAGCTCATT
TTCCCTAGTCCGCCTTCGTTCAAGGGTCAGGAATGGACCAGAACAGATGG
GTTCTGGAGGCCCCTGAACAGAGGGCTATGGCTGTGGAGAAGGTTCTTGG
CCCGTTGGACTCACACAGACCCTGTACCCTCTCGGCAAGCATCTTCAGTC
AGATTATCCTCAGTTTCAGATACTTCATAATACCTTGTGTTGTGTGGGGT
CATACATCATCGTGTTTGTAAGAGAAGATGGTCATTTTATTCTCTGTATA
AAACTTAGCTCTAAAGCAGAAACTAAAGCAGCAAATGCAGGAAGGCTGTC
TCGCCATCCTCAAGACTCAGCAGCTCTCATTCTCCAGTGGTGAGCACACC
ATTTGTGCTGCTGCTGTTGTCGTGAAATATAATAACAGTGGAAGTCACAA
AAATGTCCCCTGCCCAGCCCCCTCGCCGCCCTTGACCTCCTGCAGGCCAT
GTGTGTATTACTTGTCTAGTGATGTCCTCTCAAAGTGCTGTACGCGAGCT
CGGCGCCACCTCCGCCTCCCTTTCAGAGCCTGCTCCCCGCCCTCTCTGCT
CGCTGCATTGTGGTGTTCTCTTCTCAAGGCTTTGAAATCTCCCCTTGCAC
TGAGATTAGTCGTCAGATCTCTCCCCGTCTCCCTCCCAACTTATACGACC
TGATTTCCTTAGGACGGAACCGCAGGCACCTGCGCCGGGCGTCTTACTCC
CGCTGCTTGTTCTGTCCCCTCCCTCGGACCAAACAGTGCTCATGCTTCAG
GACCTTGTTTGTCGAAGATGTTGGTTTCCCTTTCTCTGTTATTTATATAA
AAATAATTTATCAAAAGGATATTTTAAAAAAGCTAGTCTGTCTTGAAACT
TGTTTACCTTAAAATTATCAGAATCTCAGTGTTTGAAAGTACTGAAGCAC
AAACATATATCATCTCTGTACCATTCTGTACTAAAGCACTTGAGTCTAAT
AAATAAAGAAATCAGCACCCCT

NM 003165.6 Homo sapiens syntaxin binding protein 1 (STXBP1), transcript variant 1, mRNA (SEQ ID NO: 12)

AGTCGGTCCCTAGCGCGGCTGCGGGGCGGAGAGCTGCGGCTGGCCCAGCG
CGCCCACCTGAGGAGGCGGCGGGGTCCGCAGGCGTCGCGGGACGAGGAGA
TCGGAGCCGGGAGACTCGCGCAGCGCCATGGCCCCCATTGGCCTCAAAGC
TGTTGTCGGAGAGAAGATTATGCATGATGTGATAAAGAAGGTCAAGAAGA
AGGGGGAATGGAAGGTGCTGGTGGTGGATCAGTTAAGCATGAGGATGCTG
TCCTCCTGCTGCAAGATGACAGACATCATGACCGAGGGCATAACGATTGT
GGAAGATATCAATAAGCGCAGAGAGCCGCTCCCCAGCCTGGAGGCTGTGT
ATCTCATCACTCCATCCGAGAAGTCCGTCCACTCTCTCATCAGTGACTTT
AAGGACCCGCCGACTGCTAAATACCGGGCTGCACACGTCTTCTTCACTGA
CTCTTGTCCAGATGCCCTGTTTAATGAACTGGTAAAATCCCGAGCAGCCA
AAGTCATCAAAACTCTGACGGAAATCAATATTGCATTTCTCCCGTATGAA
TCCCAGGTCTATTCCTTGGACTCTGCTGACTCTTTCCAAAGCTTCTACAG
TCCCCACAAGGCTCAGATGAAGAATCCTATACTGGAGCGCCTGGCAGAGC
AGATCGCGACCCTTTGTGCCACCCTGAAGGAGTACCCGGCTGTGCGGTAT
CGGGGGGAATACAAGGACAATGCCCTGCTGGCTCAGCTAATCCAGGACAA
GCTCGATGCCTATAAAGCTGATGATCCAACAATGGGGGAGGGCCCAGACA
AGGCACGCTCCCAGCTCCTGATCCTGGATCGAGGCTTTGACCCCAGCTCC
CCTGTGCTCCATGAATTGACTTTTCAGGCTATGAGTTATGATCTGCTGCC
TATCGAAAATGATGTATACAAGTATGAGACCAGCGGCATCGGGGAGGCAC
GGGTGAAGGAGGTGCTCCTGGACGAGGACGACGACCTGTGGATAGCACTG
CGCCACAAGCACATCGCAGAGGTGTCCCAGGAAGTCACCCGGTCTCTGAA
AGATTTTTCTTCTAGCAAGAGAATGAATACTGGAGAGAAGACCACCATGC
GGGACCTGTCCCAGATGCTGAAGAAGATGCCTCAGTACCAGAAAGAGCTC
AGCAAGTACTCCACCCACCTGCACCTTGCTGAGGACTGTATGAAGCATTA
CCAAGGCACCGTAGACAAACTCTGCCGAGTGGAGCAGGACCTGGCCATGG
GCACAGATGCTGAGGGAGAGAAGATCAAGGACCCTATGCGAGCCATCGTC
CCCATTCTGCTGGATGCCAATGTCAGCACTTATGACAAAATCCGCATCAT
CCTTCTCTACATCTTTTTGAAGAATGGCATCACGGAGGAAAACCTGAACA
AACTGATCCAGCACGCCCAGATACCCCCGGAGGATAGTGAGATCATCACC
AACATGGCTCACCTCGGCGTGCCCATCGTCACCGATTCCACGCTGCGTCG
CCGGAGCAAGCCGGAGCGGAAGGAACGCATCAGCGAGCAGACCTACCAGC
TCTCACGGTGGACTCCGATTATCAAGGACATCATGGAGGACACTATTGAG
GACAAACTTGACACCAAACACTACCCTTATATCTCTACCCGTTCCTCTGC
CTCCTTCAGCACCACCGCCGTCAGCGCCCGCTATGGGCACTGGCATAAGA
ACAAGGCCCCAGGCGAGTACCGCAGTGGCCCCCGCCTCATCATTTTCATC
CTTGGGGGTGTGAGCCTGAATGAGATGCGCTGCGCCTACGAGGTGACCCA
GGCCAACGGAAAGTGGGAGGTGCTGATAGGTTCTACTCACATTCTTACTC
CCACCAAATTTCTCATGGACCTGAGACACCCCGACTTCAGGGAGTCCTCT
AGGGTATCTTTTGAGGATCAGGCTCCAACAATGGAGTGAGAGCCAAAGAA
ACAAAGATCCACACACATCCTCACCCCACAGAAACTGCTGGACACACTGA
AGAAACTGAATAAAACAGATGAAGAAATAAGCAGTTAAAAAAATAAGTCG
CCCCTCCAAAACACGCCCCCATCCCACAGCGCTCCGCAGCTTCCCACCAC
CGCCCGCCTCAGTTCCTTTGCGTCTGTTGCCTCCCCAGCCCTGCACGCCC
TGGCTGGCACTGTTGCCGCTGCATTCTCGTGTTCAGTGATGCCCTCTTCT
TGTTTGAAACAAAAGAAAATAATGCATTGTGTTTTTTAAAAAGAGTATCT
TATACATGTATCCTAAAAAGAGAAGCTCATGTGCAATTGGTGCACAGCAG
GAGAAATTTCTGGACTGTTAGGATGAATGGACGCCTTCTCCCCGTTATTT
AAGATTTGTGACCTTGTACATAACCCTGGGTGACGTGCACATTGCTTGGG
TATGGAACGGTAGAAATTTGGGTGTTTTTAAAACCTTGTTTGGGGTTGTT
CCTGTCCTTGTTGAGAATCATAGAGATGTCTGTGTTCTTGGAGTATTTCA
CACTGAGGACTAATCTGCTATCTTCATTCCAGTCCCTACCCCTCAGTGCC
TGCTCTCATCCAAATAACCTGGGAGGTGACAATCAGGATATCTCAGGAGG
TCCAAGGTGGAACAGACCTCTTTGCCTTTCCCAGCGTCTCATACCCCCGG
TAGTGCAGCTGTGGGTGGAGGCTGGGGTGTCTGCACGAAGTCAGGCCAGC
GTCCTCCTCCACAGCCTGTCACTGCCCCCTCCCCAGCCTGTGTCCACAGT
GCTGTGATCCCGAGGGAAGTCCTCCAGTCTAAGTCACAGTGCCCTGACAG
GTGAGAAGCAAACTCCCGCTGGAAGCCTCCATCTCTTTGGAAAAACAGTT
AGTCTGGAGCCTGTGGCCCAGGCCCTTCTGTCCCCAGGCATCATCCCAAC
AGCTCATTTTCCCTAGTCCGCCTTCGTTCAAGGGTCAGGAATGGACCAGA
ACAGATGGGTTCTGGAGGCCCCTGAACAGAGGGCTATGGCTGTGGAGAAG
GTTCTTGGCCCGTTGGACTCACACAGACCCTGTACCCTCTCGGCAAGCAT
CTTCAGTCAGATTATCCTCAGTTTCAGATACTTCATAATACCTTGTGTTG
TGTGGGGTCATACATCATCGTGTTTGTAAGAGAAGATGGTCATTTTATTC
TCTGTATAAAACTTAGCTCTAAAGCAGAAACTAAAGCAGCAAATGCAGGA
AGGCTGTCTCGCCATCCTCAAGACTCAGCAGCTCTCATTCTCCAGTGGTG
AGCACACCATTTGTGCTGCTGCTGTTGTCGTGAAATATAATAACAGTGGA
AGTCACAAAAATGTCCCCTGCCCAGCCCCCTCGCCGCCCTTGACCTCCTG
CAGGCCATGTGTGTATTACTTGTCTAGTGATGTCCTCTCAAAGTGCTGTA
CGCGAGCTCGGCGCCACCTCCGCCTCCCTTTCAGAGCCTGCTCCCCGCCC
TCTCTGCTCGCTGCATTGTGGTGTTCTCTTCTCAAGGCTTTGAAATCTCC
CCTTGCACTGAGATTAGTCGTCAGATCTCTCCCCGTCTCCCTCCCAACTT
ATACGACCTGATTTCCTTAGGACGGAACCGCAGGCACCTGCGCCGGGCGT
CTTACTCCCGCTGCTTGTTCTGTCCCCTCCCTCGGACCAAACAGTGCTCA
TGCTTCAGGACCTTGTTTGTCGAAGATGTTGGTTTCCCTTTCTCTGTTAT
TTATATAAAAATAATTTATCAAAAGGATATTTTAAAAAAGCTAGTCTGTC
TTGAAACTTGTTTACCTTAAAATTATCAGAATCTCAGTGTTTGAAAGTAC
TGAAGCACAAACATATATCATCTCTGTACCATTCTGTACTAAAGCACTTG
AGTCTAATAAATAAAGAAATCAGCACCCCT

NM 001348250.2 Homo sapiens solute carrier family 6 member 1 (SLC6A1), transcript variant 2, mRNA (SEQ ID NO: 13)

GCCCTCGGAAGACCGAGACAGCGGAGAGGTTGCGGGTGAGCTGCGCTGAG
CCCAGGAGCCGAGGAGTCGGGAGCGCAGTAGCGCTGAGCCCGAGCCCGAG
CGGCCCCGCGTCCCGAGCGCATCGGAGCGGCCGAGCCGCCCGGATGCAGC
GCCTGTCCCGGGCAGCGCAGCCCCGGCCGCAGGATCTCACCCAGGGTGGC
AGAAGGAGGCCTTCTGGAGCTGACCCACCCCCGACGACCATCAGGGTGCC
CTTGAGCCGCAAAACTGCTGTCCACGTGGACCGGGGGTGACATCGCACGT
CCATCTGCCAGGACCCCTGCGTCCAAATTCCGAGACATGGCGACCAACGG
CAGCAAGGTGGCCGACGGGCAGATCTCCACCGAGGTCAGCGAGGCCCCTG
TGGCCAATGACAAGCCCAAAACCTTGGTGGTCAAGGTGCAGAAGAAGGCG
GCAGACCTCCCCGACCGGGACACGTGGAAGGGCCGCTTCGACTTCCTCAT
GTCCTGTGTGGGCTATGCCATCGGCCTGGGCAACGTCTGGAGGTTCCCCT
ATCTCTGCGGGAAAAATGGTGGGGGAGCCTTCCTGATCCCCTATTTCCTG
ACACTCATCTTTGCGGGGGTCCCACTCTTCCTGCTGGAGTGCTCCCTGGG
CCAGTACACCTCCATCGGGGGGCTAGGGGTATGGAAGCTGGCTCCTATGT
TCAAGGGCGTGGGCCTTGCGGCTGCTGTGCTATCATTCTGGCTGAACATC
TACTACATCGTCATCATCTCCTGGGCCATTTACTACCTGTACAACTCCTT
CACCACGACACTGCCGTGGAAACAGTGCGACAACCCCTGGAACACAGACC
GCTGCTTCTCCAACTACAGCATGGTCAACACTACCAACATGACCAGCGCT
GTGGTGGAGTTCTGGGAGCGCAACATGCATCAGATGACGGACGGGCTGGA
TAAGCCAGGTCAGATCCGCTGGCCACTGGCCATCACGCTGGCCATCGCCT
GGATCCTTGTGTATTTCTGTATCTGGAAGGGTGTTGGCTGGACTGGAAAG
GTGGTCTACTTTTCAGCCACATACCCCTACATCATGCTGATCATCCTGTT
CTTCCGTGGAGTGACGCTGCCCGGGGCCAAGGAGGGCATCCTCTTCTACA
TCACACCCAACTTCCGCAAGCTGTCTGACTCCGAGGTGTGGCTGGATGCG
GCAACCCAGATCTTCTTCTCATACGGGCTGGGCCTGGGGTCCCTGATCGC
TCTCGGGAGCTACAACTCTTTCCACAACAATGTCTACAGGGACTCCATCA
TCGTCTGCTGCATCAATTCGTGCACCAGCATGTTCGCAGGATTCGTCATC
TTCTCCATCGTGGGCTTCATGGCCCATGTCACCAAGAGGTCCATTGCTGA
TGTGGCGGCCTCAGGCCCCGGGCTGGCGTTCCTGGCATACCCAGAGGCGG
TGACCCAGCTGCCTATCTCCCCACTCTGGGCCATCCTCTTCTTCTCCATG
CTGTTGATGCTGGGCATTGACAGCCAGTTCTGCACTGTGGAGGGCTTCAT
CACAGCCCTGGTGGATGAGTACCCCAGGCTCCTCCGCAACCGCAGAGAGC
TCTTCATTGCTGCTGTCTGCATCATCTCCTACCTGATCGGTCTCTCTAAC
ATCACTCAGGGGGGTATTTATGTCTTCAAACTCTTTGACTACTACTCTGC
CAGTGGCATGAGCCTGCTGTTCCTCGTGTTCTTTGAATGTGTCTCTATTT
CCTGGTTTTACGGTGTCAACCGATTCTATGACAATATCCAAGAGATGGTT
GGATCCAGGCCCTGCATCTGGTGGAAACTCTGCTGGTCTTTCTTCACACC
AATCATTGTGGCGGGCGTGTTCATTTTCAGTGCTGTGCAGATGACGCCAC
TCACCATGGGAAACTATGTTTTCCCCAAGTGGGGCCAGGGTGTGGGCTGG
CTGATGGCTCTGTCTTCCATGGTCCTCATCCCCGGGTACATGGCCTACAT
GTTCCTCACCTTAAAGGGCTCCCTGAAGCAGCGCATCCAAGTCATGGTCC
AGCCCAGCGAAGACATCGTTCGCCCAGAGAATGGTCCTGAGCAGCCCCAG
GCGGGCAGCTCCACCAGCAAGGAGGCCTACATCTAGGGTGGGGGCCACTC
ACCGACCCGACACTCTCACCCCCCGACCTGGCTGAGTGCGACCACCACTT
GATGTCTGAGGATACCTTCCATCTCAACCTACCTCGAGTGGCGAGTCCAG
ACACCATCACCACGCAGAGAGGGGAGGTGGGAGGACAGTTAGACCCCTGG
GTGGGCCCTGCCGTGGGCAAGGATACCCGGTGGCTTCTGGCACCTGGCGG
GCTGGTGACCTTTTTAATCCAGGCCCCATCAGCATCCCACGATCGGCCTT
GGTAACCGCCGCGGTAGATCATTTTTATCCCGCCAGGGAGTGTGATGCAG
GAAGACCACATGCGCTCCTGGCTTTTAAACCTGTTCCTGACTGTTCTCTT
ACTGCCGAAACCCTTGACTGTTATCTCGGACTTTGCAGGAGTTCCTTTCC
CTCCGAACGCTGCTCCATGCACAGGAAAAGGGCATTTTGTACAATGGGGA
CTTCCCGGGAACGCTTGCTCTTAAGTACCAGAAGCCGGCGGAGCTCTGGC
TTTCGTGTTTTTGGTTTTCTCCTTCCCAAGGCAGCTGGATTGAAAAAACA
AAACAAAACAAAAAAACCCAGGGGCGTCAGTCGATATTCCCAGGGCCGCT
TCTCCTGCAGTCTGTGGAGCGTCCTTGTCCCCGCCGCCGGAATGAATGAG
CATTCTGCAGCCCGATGTCCCTGTCCCCTCCTCGCCGGGCCATTCTGATT
GGACCTGGCCCAGTGCAATCTGTCCAGACAAGCCCTGCTTGCTGGAAAAC
TGCCACAAGCACAATTGATCTCTTTTTATCGCCATTCCAGGGGCCTCAGG
TCCTACTGGGGAAACTTCCTATACCGGAGCTCCAGTTTCTCTTAAGCTGC
CCAATTTCACAGAGTACAAAATAGTTGTAGGGGAAATCAAGGTGAAGGAT
CTGTCCGACAGTCAAGACGGATCCACAGTAATCTTTCGGTCTCCTTAAAC
TACCACCCTCGCTGCCACCCACCCCAAGCTGCTGCCGCCTCACCTTCCTT
GAAATTTCTCAGCGGGAGTCTCCTCACTGCCACTAAAATCCACCCAGCCC
ACTAACTGAGGAGCTAGTGTTAATCCAGAGAACCCCCCGCAATGTGCTTC
CGAGATTCAGACTGCTTCATTGGGAAGTATGATTTGTTCCTTTCTGGAAT
TGGGCTCCGTGGTGGCGGCGGCACTTCAAGCAAAGACAGTTTCTTGCAAG
CTCCAGTAGCTCCGCGTGTCTCATTTGCCAGGAAGATGGGTTCCCACGTA
GCAAATCGTACATTGTGCCCTGTAGCTCCTTAGCTAGTTAGCTCACAAGC
CGTGTTTTATGACTAATCCTTAATAACTATGGTAAATAACTGTGACTGTG
GGGTTTTTAATCTCTTGTCATTCTCATCCAAAAGTGACCAGCATACCAGT
TCTTGCAATAAGATATTACCCTCAGAATATTAAGCACATTATTGTAGAGA
AAAAAAAATATGTGTACACATATGAACGCACAACATGCACATTCATCCTC
ACATGTGGCACGTAAGGTCTCATTTGATATTGTGTAGGAAATCTGAAGCC
TTTTCCTGAGGTCATCTGTAAAATAGTCTCATTGCCAAGGCATCCCCAGT
GCCAGCTGGTGAATCCATGATCAAAATGCATACGTATTGTTAAATGATAA
GGTTTAGAATGACAGGAACCCATCACTGTGTCTCATGGTCCCACTTCCCC
ATCTGTGTGTGAATTCCTTTAGACTAAGGGCAGGAAGACTTCCAGCTTTC
TCTTTGTTCTTCAATGTGAAACTGAGACCAAGTCTCTCTAAGACAAATGC
AGTGTATTTAATGTTTGTAAGCAATTCTAAGTGAGATGTTTGGCAAGAAA
TCCCCTAACTGATTTCCATCCAAACCTACCTTATAGAGCACAATATTAAG
TGTTGTACAATTACTGTGAGAACTGTGAATATGTGTAACTTTTTTTTAGT
ATTTGCCCGGGGGGAAAAAGATATTGTATTATCATATATGCTTTTTTGCA
ATAAGGATTTATTCTCAGAACACCAAGTAAATCTATCTCTATATAAAAAA
TATATGTAATATATACATATTCAAAGTATATACAGAGCCTGTTTTAAAAA
ATACAGTATTATTTAGTAAAATTATCTGTTCTATGGACCAAATGTAAAAT
ATTTATAAATGAAGATGCATTTTAAATGTCTATAAATGGTGTCATAACTA
GAGCACGGGCGTTATGTAAGTTTCTAAGAATTTAGAGGATAAATAATAAA
GGTTCTATGATATACAA

NM 001348251.2 Homo sapiens solute carrier family 6 member 1 (SLC6A1), transcript variant 3, mRNA (SEQ ID NO: 14)

GCCCTCGGAAGACCGAGACAGCGGAGAGGTTGCGGGTGAGCTGCGCTGAG
CCCAGGAGCCGAGGAGTCGGGAGCGCAGTAGCGCTGAGCCCGAGCCCGAG
CGGCCCCGCGTCCCGAGCGCATCGGAGCGGCCGAGCCGCCCGGATGCAGC
GCCTGTCCCGGGCAGCGCAGCCCCGGCCGCAGGATCTCACCCAGGGTGGC
AGAAGGAGGCCTTCTGGAGCTGACCCACCCCCGACGACCATCAGGGTGCC
CTTGAGCCGCAAAACTGCTGTCCACGTGGACCGGGGGTGACATCGCACGT
CCATCTGCCAGGACCCCTGCGTCCAAATTCCGAGACATGGCGACCAACGG
CAGCAAGGTGGCCGACGGGCAGATCTCCACCGAGGAGCCTTCCTGATCCC
CTATTTCCTGACACTCATCTTTGCGGGGGTCCCACTCTTCCTGCTGGAGT
GCTCCCTGGGCCAGTACACCTCCATCGGGGGGCTAGGGGTATGGAAGCTG
GCTCCTATGTTCAAGGGCGTGGGCCTTGCGGCTGCTGTGCTATCATTCTG
GCTGAACATCTACTACATCGTCATCATCTCCTGGGCCATTTACTACCTGT
ACAACTCCTTCACCACGACACTGCCGTGGAAACAGTGCGACAACCCCTGG
AACACAGACCGCTGCTTCTCCAACTACAGCATGGTCAACACTACCAACAT
GACCAGCGCTGTGGTGGAGTTCTGGGAGCGCAACATGCATCAGATGACGG
ACGGGCTGGATAAGCCAGGTCAGATCCGCTGGCCACTGGCCATCACGCTG
GCCATCGCCTGGATCCTTGTGTATTTCTGTATCTGGAAGGGTGTTGGCTG
GACTGGAAAGGTGGTCTACTTTTCAGCCACATACCCCTACATCATGCTGA
TCATCCTGTTCTTCCGTGGAGTGACGCTGCCCGGGGCCAAGGAGGGCATC
CTCTTCTACATCACACCCAACTTCCGCAAGCTGTCTGACTCCGAGGTGTG
GCTGGATGCGGCAACCCAGATCTTCTTCTCATACGGGCTGGGCCTGGGGT
CCCTGATCGCTCTCGGGAGCTACAACTCTTTCCACAACAATGTCTACAGG
GACTCCATCATCGTCTGCTGCATCAATTCGTGCACCAGCATGTTCGCAGG
ATTCGTCATCTTCTCCATCGTGGGCTTCATGGCCCATGTCACCAAGAGGT
CCATTGCTGATGTGGCGGCCTCAGGCCCCGGGCTGGCGTTCCTGGCATAC
CCAGAGGCGGTGACCCAGCTGCCTATCTCCCCACTCTGGGCCATCCTCTT
CTTCTCCATGCTGTTGATGCTGGGCATTGACAGCCAGTTCTGCACTGTGG
AGGGCTTCATCACAGCCCTGGTGGATGAGTACCCCAGGCTCCTCCGCAAC
CGCAGAGAGCTCTTCATTGCTGCTGTCTGCATCATCTCCTACCTGATCGG
TCTCTCTAACATCACTCAGGGGGGTATTTATGTCTTCAAACTCTTTGACT
ACTACTCTGCCAGTGGCATGAGCCTGCTGTTCCTCGTGTTCTTTGAATGT
GTCTCTATTTCCTGGTTTTACGGTGTCAACCGATTCTATGACAATATCCA
AGAGATGGTTGGATCCAGGCCCTGCATCTGGTGGAAACTCTGCTGGTCTT
TCTTCACACCAATCATTGTGGCGGGCGTGTTCATTTTCAGTGCTGTGCAG
ATGACGCCACTCACCATGGGAAACTATGTTTTCCCCAAGTGGGGCCAGGG
TGTGGGCTGGCTGATGGCTCTGTCTTCCATGGTCCTCATCCCCGGGTACA
TGGCCTACATGTTCCTCACCTTAAAGGGCTCCCTGAAGCAGCGCATCCAA
GTCATGGTCCAGCCCAGCGAAGACATCGTTCGCCCAGAGAATGGTCCTGA
GCAGCCCCAGGCGGGCAGCTCCACCAGCAAGGAGGCCTACATCTAGGGTG
GGGGCCACTCACCGACCCGACACTCTCACCCCCCGACCTGGCTGAGTGCG
ACCACCACTTGATGTCTGAGGATACCTTCCATCTCAACCTACCTCGAGTG
GCGAGTCCAGACACCATCACCACGCAGAGAGGGGAGGTGGGAGGACAGTT
AGACCCCTGGGTGGGCCCTGCCGTGGGCAAGGATACCCGGTGGCTTCTGG
CACCTGGCGGGCTGGTGACCTTTTTAATCCAGGCCCCATCAGCATCCCAC
GATCGGCCTTGGTAACCGCCGCGGTAGATCATTTTTATCCCGCCAGGGAG
TGTGATGCAGGAAGACCACATGCGCTCCTGGCTTTTAAACCTGTTCCTGA
CTGTTCTCTTACTGCCGAAACCCTTGACTGTTATCTCGGACTTTGCAGGA
GTTCCTTTCCCTCCGAACGCTGCTCCATGCACAGGAAAAGGGCATTTTGT
ACAATGGGGACTTCCCGGGAACGCTTGCTCTTAAGTACCAGAAGCCGGCG
GAGCTCTGGCTTTCGTGTTTTTGGTTTTCTCCTTCCCAAGGCAGCTGGAT
TGAAAAAACAAAACAAAACAAAAAAACCCAGGGGCGTCAGTCGATATTCC
CAGGGCCGCTTCTCCTGCAGTCTGTGGAGCGTCCTTGTCCCCGCCGCCGG
AATGAATGAGCATTCTGCAGCCCGATGTCCCTGTCCCCTCCTCGCCGGGC
CATTCTGATTGGACCTGGCCCAGTGCAATCTGTCCAGACAAGCCCTGCTT
GCTGGAAAACTGCCACAAGCACAATTGATCTCTTTTTATCGCCATTCCAG
GGGCCTCAGGTCCTACTGGGGAAACTTCCTATACCGGAGCTCCAGTTTCT
CTTAAGCTGCCCAATTTCACAGAGTACAAAATAGTTGTAGGGGAAATCAA
GGTGAAGGATCTGTCCGACAGTCAAGACGGATCCACAGTAATCTTTCGGT
CTCCTTAAACTACCACCCTCGCTGCCACCCACCCCAAGCTGCTGCCGCCT
CACCTTCCTTGAAATTTCTCAGCGGGAGTCTCCTCACTGCCACTAAAATC
CACCCAGCCCACTAACTGAGGAGCTAGTGTTAATCCAGAGAACCCCCCGC
AATGTGCTTCCGAGATTCAGACTGCTTCATTGGGAAGTATGATTTGTTCC
TTTCTGGAATTGGGCTCCGTGGTGGCGGCGGCACTTCAAGCAAAGACAGT
TTCTTGCAAGCTCCAGTAGCTCCGCGTGTCTCATTTGCCAGGAAGATGGG
TTCCCACGTAGCAAATCGTACATTGTGCCCTGTAGCTCCTTAGCTAGTTA
GCTCACAAGCCGTGTTTTATGACTAATCCTTAATAACTATGGTAAATAAC
TGTGACTGTGGGGTTTTTAATCTCTTGTCATTCTCATCCAAAAGTGACCA
GCATACCAGTTCTTGCAATAAGATATTACCCTCAGAATATTAAGCACATT
ATTGTAGAGAAAAAAAAATATGTGTACACATATGAACGCACAACATGCAC
ATTCATCCTCACATGTGGCACGTAAGGTCTCATTTGATATTGTGTAGGAA
ATCTGAAGCCTTTTCCTGAGGTCATCTGTAAAATAGTCTCATTGCCAAGG
CATCCCCAGTGCCAGCTGGTGAATCCATGATCAAAATGCATACGTATTGT
TAAATGATAAGGTTTAGAATGACAGGAACCCATCACTGTGTCTCATGGTC
CCACTTCCCCATCTGTGTGTGAATTCCTTTAGACTAAGGGCAGGAAGACT
TCCAGCTTTCTCTTTGTTCTTCAATGTGAAACTGAGACCAAGTCTCTCTA
AGACAAATGCAGTGTATTTAATGTTTGTAAGCAATTCTAAGTGAGATGTT
TGGCAAGAAATCCCCTAACTGATTTCCATCCAAACCTACCTTATAGAGCA
CAATATTAAGTGTTGTACAATTACTGTGAGAACTGTGAATATGTGTAACT
TTTTTTTAGTATTTGCCCGGGGGGAAAAAGATATTGTATTATCATATATG
CTTTTTTGCAATAAGGATTTATTCTCAGAACACCAAGTAAATCTATCTCT
ATATAAAAAATATATGTAATATATACATATTCAAAGTATATACAGAGCCT
GTTTTAAAAAATACAGTATTATTTAGTAAAATTATCTGTTCTATGGACCA
AATGTAAAATATTTATAAATGAAGATGCATTTTAAATGTCTATAAATGGT
GTCATAACTAGAGCACGGGCGTTATGTAAGTTTCTAAGAATTTAGAGGAT
AAATAATAAAGGTTCTATGATATACAA

NM 001348252.2 Homo sapiens solute carrier family 6 member 1 (SLC6A1), transcript variant 4, mRNA (SEQ ID NO: 15)

GCCCTCGGAAGACCGAGACAGCGGAGAGGTTGCGGGTGAGCTGCGCTGAG
CCCAGGAGCCGAGGAGTCGGGAGCGCAGTAGCGCTGAGCCCGAGCCCGAG
CGGCCCCGCGTCCCGAGCGCATCGGAGCGGCCGAGCCGCCCGGATGCAGC
GCCTGTCCCGGGCAGCGCAGCCCCGGCCGCAGGATCTCACCCAGGGTGGC
AGAAGGAGGCCTTCTGGAGCTGACCCACCCCCGACGACCATCAGGGTGAG
GCAACTCCAAGGTCCTACTCTCTTTCTGTGCCTGTTACCCACCCCGTCCT
CCTAGGGTGCCCTTGAGCCGCAAAACTGCTGTCCACGTGGACCGGGGGTG
ACATCGCACGTCCATCTGCCAGGACCCCTGCGTCCAAATTCCGAGACATG
GCGACCAACGGCAGCAAGGTGGCCGACGGGCAGATCTCCACCGAGGCGTG
GGCCTTGCGGCTGCTGTGCTATCATTCTGGCTGAACATCTACTACATCGT
CATCATCTCCTGGGCCATTTACTACCTGTACAACTCCTTCACCACGACAC
TGCCGTGGAAACAGTGCGACAACCCCTGGAACACAGACCGCTGCTTCTCC
AACTACAGCATGGTCAACACTACCAACATGACCAGCGCTGTGGTGGAGTT
CTGGGAGCGCAACATGCATCAGATGACGGACGGGCTGGATAAGCCAGGTC
AGATCCGCTGGCCACTGGCCATCACGCTGGCCATCGCCTGGATCCTTGTG
TATTTCTGTATCTGGAAGGGTGTTGGCTGGACTGGAAAGGTGGTCTACTT
TTCAGCCACATACCCCTACATCATGCTGATCATCCTGTTCTTCCGTGGAG
TGACGCTGCCCGGGGCCAAGGAGGGCATCCTCTTCTACATCACACCCAAC
TTCCGCAAGCTGTCTGACTCCGAGGTGTGGCTGGATGCGGCAACCCAGAT
CTTCTTCTCATACGGGCTGGGCCTGGGGTCCCTGATCGCTCTCGGGAGCT
ACAACTCTTTCCACAACAATGTCTACAGGGACTCCATCATCGTCTGCTGC
ATCAATTCGTGCACCAGCATGTTCGCAGGATTCGTCATCTTCTCCATCGT
GGGCTTCATGGCCCATGTCACCAAGAGGTCCATTGCTGATGTGGCGGCCT
CAGGCCCCGGGCTGGCGTTCCTGGCATACCCAGAGGCGGTGACCCAGCTG
CCTATCTCCCCACTCTGGGCCATCCTCTTCTTCTCCATGCTGTTGATGCT
GGGCATTGACAGCCAGTTCTGCACTGTGGAGGGCTTCATCACAGCCCTGG
TGGATGAGTACCCCAGGCTCCTCCGCAACCGCAGAGAGCTCTTCATTGCT
GCTGTCTGCATCATCTCCTACCTGATCGGTCTCTCTAACATCACTCAGGG
GGGTATTTATGTCTTCAAACTCTTTGACTACTACTCTGCCAGTGGCATGA
GCCTGCTGTTCCTCGTGTTCTTTGAATGTGTCTCTATTTCCTGGTTTTAC
GGTGTCAACCGATTCTATGACAATATCCAAGAGATGGTTGGATCCAGGCC
CTGCATCTGGTGGAAACTCTGCTGGTCTTTCTTCACACCAATCATTGTGG
CGGGCGTGTTCATTTTCAGTGCTGTGCAGATGACGCCACTCACCATGGGA
AACTATGTTTTCCCCAAGTGGGGCCAGGGTGTGGGCTGGCTGATGGCTCT
GTCTTCCATGGTCCTCATCCCCGGGTACATGGCCTACATGTTCCTCACCT
TAAAGGGCTCCCTGAAGCAGCGCATCCAAGTCATGGTCCAGCCCAGCGAA
GACATCGTTCGCCCAGAGAATGGTCCTGAGCAGCCCCAGGCGGGCAGCTC
CACCAGCAAGGAGGCCTACATCTAGGGTGGGGGCCACTCACCGACCCGAC
ACTCTCACCCCCCGACCTGGCTGAGTGCGACCACCACTTGATGTCTGAGG
ATACCTTCCATCTCAACCTACCTCGAGTGGCGAGTCCAGACACCATCACC
ACGCAGAGAGGGGAGGTGGGAGGACAGTTAGACCCCTGGGTGGGCCCTGC
CGTGGGCAAGGATACCCGGTGGCTTCTGGCACCTGGCGGGCTGGTGACCT
TTTTAATCCAGGCCCCATCAGCATCCCACGATCGGCCTTGGTAACCGCCG
CGGTAGATCATTTTTATCCCGCCAGGGAGTGTGATGCAGGAAGACCACAT
GCGCTCCTGGCTTTTAAACCTGTTCCTGACTGTTCTCTTACTGCCGAAAC
CCTTGACTGTTATCTCGGACTTTGCAGGAGTTCCTTTCCCTCCGAACGCT
GCTCCATGCACAGGAAAAGGGCATTTTGTACAATGGGGACTTCCCGGGAA
CGCTTGCTCTTAAGTACCAGAAGCCGGCGGAGCTCTGGCTTTCGTGTTTT
TGGTTTTCTCCTTCCCAAGGCAGCTGGATTGAAAAAACAAAACAAAACAA
AAAAACCCAGGGGCGTCAGTCGATATTCCCAGGGCCGCTTCTCCTGCAGT
CTGTGGAGCGTCCTTGTCCCCGCCGCCGGAATGAATGAGCATTCTGCAGC
CCGATGTCCCTGTCCCCTCCTCGCCGGGCCATTCTGATTGGACCTGGCCC
AGTGCAATCTGTCCAGACAAGCCCTGCTTGCTGGAAAACTGCCACAAGCA
CAATTGATCTCTTTTTATCGCCATTCCAGGGGCCTCAGGTCCTACTGGGG
AAACTTCCTATACCGGAGCTCCAGTTTCTCTTAAGCTGCCCAATTTCACA
GAGTACAAAATAGTTGTAGGGGAAATCAAGGTGAAGGATCTGTCCGACAG
TCAAGACGGATCCACAGTAATCTTTCGGTCTCCTTAAACTACCACCCTCG
CTGCCACCCACCCCAAGCTGCTGCCGCCTCACCTTCCTTGAAATTTCTCA
GCGGGAGTCTCCTCACTGCCACTAAAATCCACCCAGCCCACTAACTGAGG
AGCTAGTGTTAATCCAGAGAACCCCCCGCAATGTGCTTCCGAGATTCAGA
CTGCTTCATTGGGAAGTATGATTTGTTCCTTTCTGGAATTGGGCTCCGTG
GTGGCGGCGGCACTTCAAGCAAAGACAGTTTCTTGCAAGCTCCAGTAGCT
CCGCGTGTCTCATTTGCCAGGAAGATGGGTTCCCACGTAGCAAATCGTAC
ATTGTGCCCTGTAGCTCCTTAGCTAGTTAGCTCACAAGCCGTGTTTTATG
ACTAATCCTTAATAACTATGGTAAATAACTGTGACTGTGGGGTTTTTAAT
CTCTTGTCATTCTCATCCAAAAGTGACCAGCATACCAGTTCTTGCAATAA
GATATTACCCTCAGAATATTAAGCACATTATTGTAGAGAAAAAAAAATAT
GTGTACACATATGAACGCACAACATGCACATTCATCCTCACATGTGGCAC
GTAAGGTCTCATTTGATATTGTGTAGGAAATCTGAAGCCTTTTCCTGAGG
TCATCTGTAAAATAGTCTCATTGCCAAGGCATCCCCAGTGCCAGCTGGTG
AATCCATGATCAAAATGCATACGTATTGTTAAATGATAAGGTTTAGAATG
ACAGGAACCCATCACTGTGTCTCATGGTCCCACTTCCCCATCTGTGTGTG
AATTCCTTTAGACTAAGGGCAGGAAGACTTCCAGCTTTCTCTTTGTTCTT
CAATGTGAAACTGAGACCAAGTCTCTCTAAGACAAATGCAGTGTATTTAA
TGTTTGTAAGCAATTCTAAGTGAGATGTTTGGCAAGAAATCCCCTAACTG
ATTTCCATCCAAACCTACCTTATAGAGCACAATATTAAGTGTTGTACAAT
TACTGTGAGAACTGTGAATATGTGTAACTTTTTTTTAGTATTTGCCCGGG
GGGAAAAAGATATTGTATTATCATATATGCTTTTTTGCAATAAGGATTTA
TTCTCAGAACACCAAGTAAATCTATCTCTATATAAAAAATATATGTAATA
TATACATATTCAAAGTATATACAGAGCCTGTTTTAAAAAATACAGTATTA
TTTAGTAAAATTATCTGTTCTATGGACCAAATGTAAAATATTTATAAATG
AAGATGCATTTTAAATGTCTATAAATGGTGTCATAACTAGAGCACGGGCG
TTATGTAAGTTTCTAAGAATTTAGAGGATAAATAATAAAGGTTCTATGAT
ATACAA

NM 001348253.2 Homo sapiens solute carrier family 6 member 1 (SLC6A1), transcript variant 5, mRNA (SEQ ID NO: 16)

GCCCTCGGAAGACCGAGACAGCGGAGAGGTTGCGGGTGAGCTGCGCTGAG
CCCAGGAGCCGAGGAGTCGGGAGCGCAGTAGCGCTGAGCCCGAGCCCGAG
CGGCCCCGCGTCCCGAGCGCATCGGAGCGGCCGAGCCGCCCGGATGCAGC
GCCTGTCCCGGGCAGCGCAGCCCCGGCCGCAGGATCTCACCCAGGGTGGC
AGAAGGAGGCCTTCTGGAGCTGACCCACCCCCGACGACCATCAGGGTGCC
CTTGAGCCGCAAAACTGCTGTCCACGTGGACCGGGGGTGACATCGCACGT
CCATCTGCCAGGACCCCTGCGTCCAAATTCCGAGACATGGCGACCAACGG
CAGCAAGGTGGCCGACGGGCAGATCTCCACCGAGGCGTGGGCCTTGCGGC
TGCTGTGCTATCATTCTGGCTGAACATCTACTACATCGTCATCATCTCCT
GGGCCATTTACTACCTGTACAACTCCTTCACCACGACACTGCCGTGGAAA
CAGTGCGACAACCCCTGGAACACAGACCGCTGCTTCTCCAACTACAGCAT
GGTCAACACTACCAACATGACCAGCGCTGTGGTGGAGTTCTGGGAGCGCA
ACATGCATCAGATGACGGACGGGCTGGATAAGCCAGGTCAGATCCGCTGG
CCACTGGCCATCACGCTGGCCATCGCCTGGATCCTTGTGTATTTCTGTAT
CTGGAAGGGTGTTGGCTGGACTGGAAAGGTGGTCTACTTTTCAGCCACAT
ACCCCTACATCATGCTGATCATCCTGTTCTTCCGTGGAGTGACGCTGCCC
GGGGCCAAGGAGGGCATCCTCTTCTACATCACACCCAACTTCCGCAAGCT
GTCTGACTCCGAGGTGTGGCTGGATGCGGCAACCCAGATCTTCTTCTCAT
ACGGGCTGGGCCTGGGGTCCCTGATCGCTCTCGGGAGCTACAACTCTTTC
CACAACAATGTCTACAGGGACTCCATCATCGTCTGCTGCATCAATTCGTG
CACCAGCATGTTCGCAGGATTCGTCATCTTCTCCATCGTGGGCTTCATGG
CCCATGTCACCAAGAGGTCCATTGCTGATGTGGCGGCCTCAGGCCCCGGG
CTGGCGTTCCTGGCATACCCAGAGGCGGTGACCCAGCTGCCTATCTCCCC
ACTCTGGGCCATCCTCTTCTTCTCCATGCTGTTGATGCTGGGCATTGACA
GCCAGTTCTGCACTGTGGAGGGCTTCATCACAGCCCTGGTGGATGAGTAC
CCCAGGCTCCTCCGCAACCGCAGAGAGCTCTTCATTGCTGCTGTCTGCAT
CATCTCCTACCTGATCGGTCTCTCTAACATCACTCAGGGGGGTATTTATG
TCTTCAAACTCTTTGACTACTACTCTGCCAGTGGCATGAGCCTGCTGTTC
CTCGTGTTCTTTGAATGTGTCTCTATTTCCTGGTTTTACGGTGTCAACCG
ATTCTATGACAATATCCAAGAGATGGTTGGATCCAGGCCCTGCATCTGGT
GGAAACTCTGCTGGTCTTTCTTCACACCAATCATTGTGGCGGGCGTGTTC
ATTTTCAGTGCTGTGCAGATGACGCCACTCACCATGGGAAACTATGTTTT
CCCCAAGTGGGGCCAGGGTGTGGGCTGGCTGATGGCTCTGTCTTCCATGG
TCCTCATCCCCGGGTACATGGCCTACATGTTCCTCACCTTAAAGGGCTCC
CTGAAGCAGCGCATCCAAGTCATGGTCCAGCCCAGCGAAGACATCGTTCG
CCCAGAGAATGGTCCTGAGCAGCCCCAGGCGGGCAGCTCCACCAGCAAGG
AGGCCTACATCTAGGGTGGGGGCCACTCACCGACCCGACACTCTCACCCC
CCGACCTGGCTGAGTGCGACCACCACTTGATGTCTGAGGATACCTTCCAT
CTCAACCTACCTCGAGTGGCGAGTCCAGACACCATCACCACGCAGAGAGG
GGAGGTGGGAGGACAGTTAGACCCCTGGGTGGGCCCTGCCGTGGGCAAGG
ATACCCGGTGGCTTCTGGCACCTGGCGGGCTGGTGACCTTTTTAATCCAG
GCCCCATCAGCATCCCACGATCGGCCTTGGTAACCGCCGCGGTAGATCAT
TTTTATCCCGCCAGGGAGTGTGATGCAGGAAGACCACATGCGCTCCTGGC
TTTTAAACCTGTTCCTGACTGTTCTCTTACTGCCGAAACCCTTGACTGTT
ATCTCGGACTTTGCAGGAGTTCCTTTCCCTCCGAACGCTGCTCCATGCAC
AGGAAAAGGGCATTTTGTACAATGGGGACTTCCCGGGAACGCTTGCTCTT
AAGTACCAGAAGCCGGCGGAGCTCTGGCTTTCGTGTTTTTGGTTTTCTCC
TTCCCAAGGCAGCTGGATTGAAAAAACAAAACAAAACAAAAAAACCCAGG
GGCGTCAGTCGATATTCCCAGGGCCGCTTCTCCTGCAGTCTGTGGAGCGT
CCTTGTCCCCGCCGCCGGAATGAATGAGCATTCTGCAGCCCGATGTCCCT
GTCCCCTCCTCGCCGGGCCATTCTGATTGGACCTGGCCCAGTGCAATCTG
TCCAGACAAGCCCTGCTTGCTGGAAAACTGCCACAAGCACAATTGATCTC
TTTTTATCGCCATTCCAGGGGCCTCAGGTCCTACTGGGGAAACTTCCTAT
ACCGGAGCTCCAGTTTCTCTTAAGCTGCCCAATTTCACAGAGTACAAAAT
AGTTGTAGGGGAAATCAAGGTGAAGGATCTGTCCGACAGTCAAGACGGAT
CCACAGTAATCTTTCGGTCTCCTTAAACTACCACCCTCGCTGCCACCCAC
CCCAAGCTGCTGCCGCCTCACCTTCCTTGAAATTTCTCAGCGGGAGTCTC
CTCACTGCCACTAAAATCCACCCAGCCCACTAACTGAGGAGCTAGTGTTA
ATCCAGAGAACCCCCCGCAATGTGCTTCCGAGATTCAGACTGCTTCATTG
GGAAGTATGATTTGTTCCTTTCTGGAATTGGGCTCCGTGGTGGCGGCGGC
ACTTCAAGCAAAGACAGTTTCTTGCAAGCTCCAGTAGCTCCGCGTGTCTC
ATTTGCCAGGAAGATGGGTTCCCACGTAGCAAATCGTACATTGTGCCCTG
TAGCTCCTTAGCTAGTTAGCTCACAAGCCGTGTTTTATGACTAATCCTTA
ATAACTATGGTAAATAACTGTGACTGTGGGGTTTTTAATCTCTTGTCATT
CTCATCCAAAAGTGACCAGCATACCAGTTCTTGCAATAAGATATTACCCT
CAGAATATTAAGCACATTATTGTAGAGAAAAAAAAATATGTGTACACATA
TGAACGCACAACATGCACATTCATCCTCACATGTGGCACGTAAGGTCTCA
TTTGATATTGTGTAGGAAATCTGAAGCCTTTTCCTGAGGTCATCTGTAAA
ATAGTCTCATTGCCAAGGCATCCCCAGTGCCAGCTGGTGAATCCATGATC
AAAATGCATACGTATTGTTAAATGATAAGGTTTAGAATGACAGGAACCCA
TCACTGTGTCTCATGGTCCCACTTCCCCATCTGTGTGTGAATTCCTTTAG
ACTAAGGGCAGGAAGACTTCCAGCTTTCTCTTTGTTCTTCAATGTGAAAC
TGAGACCAAGTCTCTCTAAGACAAATGCAGTGTATTTAATGTTTGTAAGC
AATTCTAAGTGAGATGTTTGGCAAGAAATCCCCTAACTGATTTCCATCCA
AACCTACCTTATAGAGCACAATATTAAGTGTTGTACAATTACTGTGAGAA
CTGTGAATATGTGTAACTTTTTTTTAGTATTTGCCCGGGGGGAAAAAGAT
ATTGTATTATCATATATGCTTTTTTGCAATAAGGATTTATTCTCAGAACA
CCAAGTAAATCTATCTCTATATAAAAAATATATGTAATATATACATATTC
AAAGTATATACAGAGCCTGTTTTAAAAAATACAGTATTATTTAGTAAAAT
TATCTGTTCTATGGACCAAATGTAAAATATTTATAAATGAAGATGCATTT
TAAATGTCTATAAATGGTGTCATAACTAGAGCACGGGCGTTATGTAAGTT
TCTAAGAATTTAGAGGATAAATAATAAAGGTTCTATGATATACAA

NM 003042.4 Homo sapiens solute carrier family 6 member 1 (SLC6A1), transcript variant 1, mRNA (SEQ ID NO: 17)

GCCCTCGGAAGACCGAGACAGCGGAGAGGTTGCGGGTGAGCTGCGCTGAG
CCCAGGAGCCGAGGAGTCGGGAGCGCAGTAGCGCTGAGCCCGAGCCCGAG
CGGCCCCGCGTCCCGAGCGCATCGGAGCGGCCGAGCCGCCCGGATGCAGC
GCCTGTCCCGGGCAGCGCAGCCCCGGCCGCAGGATCTCACCCAGGGTGGC
AGAAGGAGGCCTTCTGGAGCTGACCCACCCCCGACGACCATCAGGGTGAG
GCAACTCCAAGGTCCTACTCTCTTTCTGTGCCTGTTACCCACCCCGTCCT
CCTAGGGTGCCCTTGAGCCGCAAAACTGCTGTCCACGTGGACCGGGGGTG
ACATCGCACGTCCATCTGCCAGGACCCCTGCGTCCAAATTCCGAGACATG
GCGACCAACGGCAGCAAGGTGGCCGACGGGCAGATCTCCACCGAGGTCAG
CGAGGCCCCTGTGGCCAATGACAAGCCCAAAACCTTGGTGGTCAAGGTGC
AGAAGAAGGCGGCAGACCTCCCCGACCGGGACACGTGGAAGGGCCGCTTC
GACTTCCTCATGTCCTGTGTGGGCTATGCCATCGGCCTGGGCAACGTCTG
GAGGTTCCCCTATCTCTGCGGGAAAAATGGTGGGGGAGCCTTCCTGATCC
CCTATTTCCTGACACTCATCTTTGCGGGGGTCCCACTCTTCCTGCTGGAG
TGCTCCCTGGGCCAGTACACCTCCATCGGGGGGCTAGGGGTATGGAAGCT
GGCTCCTATGTTCAAGGGCGTGGGCCTTGCGGCTGCTGTGCTATCATTCT
GGCTGAACATCTACTACATCGTCATCATCTCCTGGGCCATTTACTACCTG
TACAACTCCTTCACCACGACACTGCCGTGGAAACAGTGCGACAACCCCTG
GAACACAGACCGCTGCTTCTCCAACTACAGCATGGTCAACACTACCAACA
TGACCAGCGCTGTGGTGGAGTTCTGGGAGCGCAACATGCATCAGATGACG
GACGGGCTGGATAAGCCAGGTCAGATCCGCTGGCCACTGGCCATCACGCT
GGCCATCGCCTGGATCCTTGTGTATTTCTGTATCTGGAAGGGTGTTGGCT
GGACTGGAAAGGTGGTCTACTTTTCAGCCACATACCCCTACATCATGCTG
ATCATCCTGTTCTTCCGTGGAGTGACGCTGCCCGGGGCCAAGGAGGGCAT
CCTCTTCTACATCACACCCAACTTCCGCAAGCTGTCTGACTCCGAGGTGT
GGCTGGATGCGGCAACCCAGATCTTCTTCTCATACGGGCTGGGCCTGGGG
TCCCTGATCGCTCTCGGGAGCTACAACTCTTTCCACAACAATGTCTACAG
GGACTCCATCATCGTCTGCTGCATCAATTCGTGCACCAGCATGTTCGCAG
GATTCGTCATCTTCTCCATCGTGGGCTTCATGGCCCATGTCACCAAGAGG
TCCATTGCTGATGTGGCGGCCTCAGGCCCCGGGCTGGCGTTCCTGGCATA
CCCAGAGGCGGTGACCCAGCTGCCTATCTCCCCACTCTGGGCCATCCTCT
TCTTCTCCATGCTGTTGATGCTGGGCATTGACAGCCAGTTCTGCACTGTG
GAGGGCTTCATCACAGCCCTGGTGGATGAGTACCCCAGGCTCCTCCGCAA
CCGCAGAGAGCTCTTCATTGCTGCTGTCTGCATCATCTCCTACCTGATCG
GTCTCTCTAACATCACTCAGGGGGGTATTTATGTCTTCAAACTCTTTGAC
TACTACTCTGCCAGTGGCATGAGCCTGCTGTTCCTCGTGTTCTTTGAATG
TGTCTCTATTTCCTGGTTTTACGGTGTCAACCGATTCTATGACAATATCC
AAGAGATGGTTGGATCCAGGCCCTGCATCTGGTGGAAACTCTGCTGGTCT
TTCTTCACACCAATCATTGTGGCGGGCGTGTTCATTTTCAGTGCTGTGCA
GATGACGCCACTCACCATGGGAAACTATGTTTTCCCCAAGTGGGGCCAGG
GTGTGGGCTGGCTGATGGCTCTGTCTTCCATGGTCCTCATCCCCGGGTAC
ATGGCCTACATGTTCCTCACCTTAAAGGGCTCCCTGAAGCAGCGCATCCA
AGTCATGGTCCAGCCCAGCGAAGACATCGTTCGCCCAGAGAATGGTCCTG
AGCAGCCCCAGGCGGGCAGCTCCACCAGCAAGGAGGCCTACATCTAGGGT
GGGGGCCACTCACCGACCCGACACTCTCACCCCCCGACCTGGCTGAGTGC
GACCACCACTTGATGTCTGAGGATACCTTCCATCTCAACCTACCTCGAGT
GGCGAGTCCAGACACCATCACCACGCAGAGAGGGGAGGTGGGAGGACAGT
TAGACCCCTGGGTGGGCCCTGCCGTGGGCAAGGATACCCGGTGGCTTCTG
GCACCTGGCGGGCTGGTGACCTTTTTAATCCAGGCCCCATCAGCATCCCA
CGATCGGCCTTGGTAACCGCCGCGGTAGATCATTTTTATCCCGCCAGGGA
GTGTGATGCAGGAAGACCACATGCGCTCCTGGCTTTTAAACCTGTTCCTG
ACTGTTCTCTTACTGCCGAAACCCTTGACTGTTATCTCGGACTTTGCAGG
AGTTCCTTTCCCTCCGAACGCTGCTCCATGCACAGGAAAAGGGCATTTTG
TACAATGGGGACTTCCCGGGAACGCTTGCTCTTAAGTACCAGAAGCCGGC
GGAGCTCTGGCTTTCGTGTTTTTGGTTTTCTCCTTCCCAAGGCAGCTGGA
TTGAAAAAACAAAACAAAACAAAAAAACCCAGGGGCGTCAGTCGATATTC
CCAGGGCCGCTTCTCCTGCAGTCTGTGGAGCGTCCTTGTCCCCGCCGCCG
GAATGAATGAGCATTCTGCAGCCCGATGTCCCTGTCCCCTCCTCGCCGGG
CCATTCTGATTGGACCTGGCCCAGTGCAATCTGTCCAGACAAGCCCTGCT
TGCTGGAAAACTGCCACAAGCACAATTGATCTCTTTTTATCGCCATTCCA
GGGGCCTCAGGTCCTACTGGGGAAACTTCCTATACCGGAGCTCCAGTTTC
TCTTAAGCTGCCCAATTTCACAGAGTACAAAATAGTTGTAGGGGAAATCA
AGGTGAAGGATCTGTCCGACAGTCAAGACGGATCCACAGTAATCTTTCGG
TCTCCTTAAACTACCACCCTCGCTGCCACCCACCCCAAGCTGCTGCCGCC
TCACCTTCCTTGAAATTTCTCAGCGGGAGTCTCCTCACTGCCACTAAAAT
CCACCCAGCCCACTAACTGAGGAGCTAGTGTTAATCCAGAGAACCCCCCG
CAATGTGCTTCCGAGATTCAGACTGCTTCATTGGGAAGTATGATTTGTTC
CTTTCTGGAATTGGGCTCCGTGGTGGCGGCGGCACTTCAAGCAAAGACAG
TTTCTTGCAAGCTCCAGTAGCTCCGCGTGTCTCATTTGCCAGGAAGATGG
GTTCCCACGTAGCAAATCGTACATTGTGCCCTGTAGCTCCTTAGCTAGTT
AGCTCACAAGCCGTGTTTTATGACTAATCCTTAATAACTATGGTAAATAA
CTGTGACTGTGGGGTTTTTAATCTCTTGTCATTCTCATCCAAAAGTGACC
AGCATACCAGTTCTTGCAATAAGATATTACCCTCAGAATATTAAGCACAT
TATTGTAGAGAAAAAAAAATATGTGTACACATATGAACGCACAACATGCA
CATTCATCCTCACATGTGGCACGTAAGGTCTCATTTGATATTGTGTAGGA
AATCTGAAGCCTTTTCCTGAGGTCATCTGTAAAATAGTCTCATTGCCAAG
GCATCCCCAGTGCCAGCTGGTGAATCCATGATCAAAATGCATACGTATTG
TTAAATGATAAGGTTTAGAATGACAGGAACCCATCACTGTGTCTCATGGT
CCCACTTCCCCATCTGTGTGTGAATTCCTTTAGACTAAGGGCAGGAAGAC
TTCCAGCTTTCTCTTTGTTCTTCAATGTGAAACTGAGACCAAGTCTCTCT
AAGACAAATGCAGTGTATTTAATGTTTGTAAGCAATTCTAAGTGAGATGT
TTGGCAAGAAATCCCCTAACTGATTTCCATCCAAACCTACCTTATAGAGC
ACAATATTAAGTGTTGTACAATTACTGTGAGAACTGTGAATATGTGTAAC
TTTTTTTTAGTATTTGCCCGGGGGGAAAAAGATATTGTATTATCATATAT
GCTTTTTTGCAATAAGGATTTATTCTCAGAACACCAAGTAAATCTATCTC
TATATAAAAAATATATGTAATATATACATATTCAAAGTATATACAGAGCC
TGTTTTAAAAAATACAGTATTATTTAGTAAAATTATCTGTTCTATGGACC
AAATGTAAAATATTTATAAATGAAGATGCATTTTAAATGTCTATAAATGG
TGTCATAACTAGAGCACGGGCGTTATGTAAGTTTCTAAGAATTTAGAGGA
TAAATAATAAAGGTTCTATGATATACAA

NM 006772.3 Homo sapiens synaptic Ras GTPase activating protein 1 (SYNGAP1), transcript variant 1, mRNA (SEQ ID NO: 18)

CTCTCTCTCGGCTGCCGCTGCTGCCGTTGGCTCTTATTCTCCTCCTCCTC
CTCCTCTCTCCTCCTCTCTGCTTCTCTCTGCTCCTCTCTCCTCCTCTCTC
CTCCTCCTCCTCCTCCACCTCCTCCTCCTTCTCCCCCTCTTTCTCCCCCT
CTTTCTCTCTTCTTTCTCCCCCGTCCCCCCGCCCCCTCCCCCCAGGCCTG
ATGAGCAGGTCTCGAGCCTCCATCCATCGGGGGAGCATCCCCGCGATGTC
CTATGCCCCCTTCAGAGATGTACGGGGACCCTCTATGCACCGAACCCAAT
ACGTTCATTCCCCGTATGATCGTCCTGGTTGGAACCCTCGGTTCTGCATC
ATCTCGGGGAACCAGCTGCTCATGCTGGATGAGGATGAGATACACCCCCT
ACTGATCCGGGACCGGAGGAGCGAGTCCAGTCGCAACAAACTGCTGAGAC
GCACAGTCTCCGTGCCGGTGGAGGGGCGGCCCCACGGCGAGCATGAATAC
CACTTGGGTCGCTCGAGGAGGAAGAGTGTCCCAGGGGGGAAGCAGTACAG
CATGGAGGGTGCCCCTGCTGCGCCCTTCCGGCCCTCGCAAGGCTTCCTGA
GCCGACGGCTAAAAAGCTCCATCAAACGAACGAAGTCACAACCCAAACTT
GACCGGACCAGCAGCTTTCGCCAGATCCTGCCTCGCTTCCGAAGTGCTGA
CCATGACCGGGCCCGGCTGATGCAAAGCTTTAAGGAGTCACACTCTCATG
AGTCCTTGCTGAGTCCTAGCAGTGCAGCTGAGGCATTGGAGCTCAACTTG
GATGAAGATTCCATTATCAAGCCAGTGCACAGCTCCATCCTGGGCCAGGA
GTTCTGTTTTGAGGTAACAACTTCATCAGGAACAAAATGCTTTGCCTGTC
GGTCTGCGGCCGAAAGAGACAAATGGATTGAGAATCTGCAGCGGGCAGTA
AAGCCCAACAAGGACAACAGCCGCCGGGTAGACAATGTGCTAAAGCTGTG
GATCATAGAGGCCCGGGAGCTGCCCCCCAAGAAGCGGTACTACTGTGAGC
TCTGCCTGGATGACATGCTGTATGCACGCACCACCTCCAAGCCCCGCTCT
GCCTCTGGGGACACCGTCTTCTGGGGCGAGCACTTCGAGTTTAACAACCT
GCCGGCTGTCCGTGCCCTGCGGCTGCATCTGTACCGTGACTCAGACAAAA
AGCGCAAGAAGGACAAGGCAGGCTATGTCGGCCTGGTGACTGTGCCAGTG
GCCACCCTGGCTGGGCGCCACTTCACAGAGCAGTGGTACCCTGTAACCCT
GCCAACAGGCAGTGGGGGATCTGGGGGCATGGGTTCGGGAGGGGGAGGGG
GCTCGGGGGGTGGCTCAGGGGGCAAGGGCAAAGGAGGTTGCCCGGCTGTG
CGGCTGAAAGCACGTTACCAGACAATGAGCATCTTGCCCATGGAGCTATA
TAAAGAGTTTGCAGAGTATGTCACCAACCATTATCGGATGCTGTGTGCAG
TCTTGGAGCCCGCCCTGAATGTCAAAGGCAAGGAGGAGGTTGCCAGTGCA
CTAGTTCACATCCTGCAGAGTACAGGCAAGGCCAAGGACTTCCTTTCAGA
CATGGCCATGTCTGAGGTAGACCGGTTCATGGAACGGGAGCACCTCATAT
TCCGCGAGAACACGCTTGCCACTAAAGCCATAGAAGAGTATATGAGACTG
ATTGGTCAGAAATACCTCAAGGATGCCATTGGAGAATTCATCCGTGCTCT
GTATGAATCTGAGGAAAACTGCGAGGTAGACCCTATCAAGTGCACAGCAT
CCAGTTTGGCAGAGCACCAGGCCAACCTGCGAATGTGCTGTGAGTTGGCC
CTGTGCAAGGTGGTCAACTCCCACTGCGTGTTCCCGAGGGAGCTGAAGGA
GGTGTTTGCTTCGTGGCGGCTGCGCTGCGCAGAGCGAGGCCGGGAGGACA
TCGCAGACAGGCTTATCAGCGCCTCACTCTTCCTGCGCTTCCTCTGCCCA
GCGATTATGTCGCCCAGTCTCTTTGGGCTTATGCAGGAGTACCCAGATGA
GCAGACCTCACGAACCCTCACCCTCATTGCCAAGGTCATCCAGAACCTGG
CCAACTTTTCCAAGTTTACCTCAAAGGAGGACTTTCTGGGCTTCATGAAT
GAGTTTCTGGAGCTGGAATGGGGTTCCATGCAGCAGTTTTTGTATGAGAT
CTCCAATCTGGACACGCTAACCAACAGCAGTAGCTTTGAGGGTTACATCG
ACTTGGGCCGAGAGCTCTCCACACTGCATGCCCTACTCTGGGAGGTGCTG
CCCCAGCTCAGCAAGGAAGCCCTCCTGAAGCTGGGTCCACTGCCCCGGCT
CCTCAACGACATCAGCACAGCTCTGAGGAACCCCAACATCCAAAGGCAGC
CAAGCCGCCAGAGTGAGCGGCCCCGGCCTCAGCCTGTGGTACTGCGGGGG
CCATCGGCTGAGATGCAGGGCTACATGATGCGGGACCTCAACAGCTCCAT
CGACCTTCAGTCCTTCATGGCTCGAGGCCTCAACAGCTCTATGGACATGG
CTCGCCTCCCCTCCCCAACCAAGGAAAAGCCACCCCCACCACCGCCTGGT
GGTGGTAAAGACCTGTTCTATGTAAGCCGTCCACCCCTGGCCCGTTCCTC
ACCAGCATACTGCACGAGCAGCTCGGACATCACAGAGCCAGAGCAGAAGA
TGCTGAGTGTCAACAAGAGTGTGTCCATGCTGGACTTACAGGGTGATGGG
CCTGGTGGCCGCCTCAACAGCAGCAGTGTTTCGAACCTGGCGGCCGTAGG
GGACCTGCTGCACTCAAGCCAGGCCTCGCTGACAGCAGCCTTGGGGCTAC
GGCCTGCGCCTGCCGGACGCCTCTCCCAGGGGAGTGGCTCATCCATCACG
GCGGCTGGCATGCGCCTCAGCCAGATGGGTGTCACCACAGACGGTGTCCC
TGCCCAGCAACTGCGAATCCCCCTCTCCTTCCAGAACCCTCTCTTCCACA
TGGCTGCTGATGGGCCAGGTCCCCCAGGCGGCCATGGAGGGGGCGGTGGC
CATGGCCCACCTTCCTCCCATCACCACCACCACCACCATCACCACCACCG
AGGTGGAGAGCCCCCTGGGGACACCTTTGCCCCATTCCATGGCTATAGCA
AGAGTGAGGACCTCTCTTCCGGGGTCCCCAAGCCCCCTGCTGCCTCCATC
CTTCATAGCCACAGCTACAGTGATGAGTTTGGACCCTCTGGCACTGACTT
CACCCGTCGGCAGCTTTCACTCCAGGACAACCTGCAGCACATGCTGTCCC
CTCCCCAGATCACCATTGGTCCCCAGAGGCCAGCCCCCTCAGGGCCTGGA
GGTGGGAGCGGTGGGGGCAGCGGTGGGGGTGGCGGGGGCCAGCCGCCTCC
ATTGCAGAGGGGCAAGTCTCAGCAGTTGACAGTCAGCGCAGCCCAGAAAC
CCCGGCCATCCAGCGGGAATCTATTGCAGTCCCCAGAGCCAAGTTATGGC
CCCGCCCGTCCACGGCAACAGAGCCTCAGCAAGGAGGGCAGCATTGGGGG
CAGCGGGGGCAGCGGTGGCGGAGGGGGTGGGGGGCTGAAGCCCTCCATCA
CCAAGCAGCATTCTCAGACACCATCCACATTGAACCCCACAATGCCAGCC
TCTGAGCGGACAGTGGCCTGGGTCTCCAACATGCCTCACCTGTCGGCTGA
CATCGAGAGTGCCCACATCGAGCGGGAAGAGTACAAGCTCAAGGAGTACT
CAAAATCGATGGATGAGAGCCGGCTGGATAGGGTGAAGGAGTACGAGGAG
GAGATTCACTCACTGAAAGAGCGGCTGCACATGTCCAACCGGAAGCTGGA
AGAGTATGAGCGGAGGCTGCTGTCCCAGGAAGAACAAACCAGCAAAATCC
TGATGCAGTATCAGGCCCGACTGGAGCAGAGTGAGAAGAGGCTAAGGCAG
CAGCAGGCAGAGAAGGATTCCCAGATCAAGAGCATCATTGGCAGGCTGAT
GCTGGTGGAGGAGGAGCTGCGCCGGGACCACCCCGCCATGGCTGAGCCGC
TGCCAGAACCCAAGAAGAGGCTGCTCGACGCTCAGGAGAGGCAGCTTCCC
CCCTTGGGTCCAACAAACCCGCGTGTGACGCTGGCCCCACCGTGGAATGG
CCTGGCCCCCCCAGCCCCACCACCCCCACCCCGGCTGCAGATTACGGAGA
ACGGCGAGTTCCGAAACACCGCAGACCACTAGCCCACCCAGCATCAGAGA
CCTTCTCTTCCTTTCCTGTGCACCCCACCCTGTAACAGCACCAACCACCA
GGATTGGACATCACCGAGGAACAGCGGGATTGCCTCCCCGAATGCCTCCC
TGGGAGGCACACTGATTGCCCACCCCCACCACTGCACCATTTCCAGGAGG
GAGAGTGGGGACCCTCAGCCGCCCCCTTTTCCTTCCCATTGGGGTGCTGC
CCTCTCTTTGACCCCCAGGGACCCTTGCCCCAGGACACCGCCTACCCCGT
ACAGACCCCTTCACTCCGGGGTGCTATCCCCATCCTCTGCCTCATCGTTC
CCCTGAGCACTGGGGGACAGACCCTCACCCCCACCCTGGGGGTGTGGCAC
CTCCAAACTTTCAACTTCAGGGTGATTTTTTTAGCAGTAACCAGAGCTGA
CAATCTAACTCCCCTCCACCGCCCCATTTTGGCCTCCCCTGCCCCCCTTG
TTATGGGGAGGGGACCCCGGGTGAGGGGGCCCTATTACCCCTTGATTTCT
CAGGAGCGTCTGGGGGGGCTCAGCACGCACAAACTCCTTCTCCTTCTACC
ACTCTTAAATTTACTCCCTCCCCACCCAGAACCCAGATGGGGTGGAGGGG
GCCACCGGGGCAGGGAGGGGGCGGCAAGGGGGGAATGGGAGTTGTCTCCC
CTTCTCCCCACACCTGATCTGCTCTCGGCTGGTCCCAGAGCGGGGTGAGG
GGGCTTATGCCCCCCCCTCCCCCAGTGTGTTGGGTGGGGTGGAATTGAGG
TTAGGGTGAGGGGTCAGGGTTTAGGAGGGTGTGTATGTTGGGAGGACAGG
CTAGTTGATCTGTCCTACTCTGACACACAGTCCCCTCTGCCCCTTCCTTC
TCTCTTCTTGGTCTCTACTCCCAGGGGGAGGGGGGAACTTACTCTAGGAA
AAGCCATGTCTCTCTCCCCCAGGGTGGGGGGACCTGTGTTGGAGGAGGGG
TGTTGGGGGGCCCCCTTCCATGACTCTGTCCCCTGGGGGAGGTAGGACAG
GGCTGGGCTTCCCTCTCATCCTCCCCCTCCCAATCTCCTTCCACCTCCCT
CCCTCCCGCCAGCTCCACGATTTTTCGGTGTTTCTCTGTACATAGTTTTC
TGGCGGGATAGGGGAGGTAGGATGGATGGGGTTTGGGGTGGGTAGGCCAT
GGGAGGGGAGAAGCCCCTCCTTGGCACCCCCTCTTCCCTGACTGCTGTCC
CCTACCCAGCCTTGCCCCCTTCATCCTTTTGCGTTTGGTATTGAGACTCT
CCTAGACTCTACTCCTCTTTCTTTTGTATGGACAGTTCCCCTTCAGTCCC
ATCCCCCTACACATACACCCAGCCGGGGCCAAATTTATACTTATATAAAA
GTTGTAAATATGTGAAATTTTATCCCTGTGCCCTTTCCCCACCTCAGGCC
CTACCCCTGGACCCTCCCCAACCTTCCTTCTCTCTTCTTTGGCTGTTGTA
ATTATCTGGGGTTTGTACTGTACATATCCGGGGTGTGTGTGTGTGGGCTG
GGGGCAACCCTTCTGTACAGAGCTTCCTGGCCCCCTCCCCCCCCGCCCCT
CTGCTTCCCTCCCCACCCACCACCTCAAGGGTAGGGAGTTGCTCTTCCTA
CCTGTTTTATTTTGTTTTCTCGTTCTCCCTCCCCACCCCACTCCCAGCCT
TATCTATCCCCCCTCACTGTCCCCTTTTCTCCACTCCCAGCCCCATTTCC
TTTTTTTCTGGAGTGTGTGGTGAAACAGAAAAAAACATGTTTAATAAACG
GAGATTGTTCTTTTA

NM 001130066.2 Homo sapiens synaptic Ras GTPase activating protein 1 (SYNGAP1), transcript variant 2, mRNA (SEQ ID NO: 19)

CTCTCTCTCGGCTGCCGCTGCTGCCGTTGGCTCTTATTCTCCTCCTCCTCCTCCTCTCTCCTCCTCTCTGCTTCTCT
CTGCTCCTCTCTCCTCCTCTCTCCTCCTCCTCCTCCTCCACCTCCTCCTCCTTCTCCCCCTCTTTCTCCCCCTCTTT
CTCTCTTCTTTCTCCCCCGTCCCCCCGCCCCCTCCCCCCAGGCCTGATGAGCAGGTCTCGAGCCTCCATCCATCGGG
GGAGCATCCCCGCGATGTCCTATGCCCCCTTCAGAGATGTACGGGGACCCTCTATGCACCGAACCCAATACGTTCAT
TCCCCGTATGATCGTCCTGGTTGGAACCCTCGGTTCTGCATCATCTCGGGGAACCAGCTGCTCATGCTGGATGAGGA
TGAGATACACCCCCTACTGATCCGGGACCGGAGGAGCGAGTCCAGTCGCAACAAACTGCTGAGACGCACAGTCTCCG
TGCCGGTGGAGGGGCGGCCCCACGGCGAGCATGAATACCACTTGGGTCGCTCGAGGAGGAAGAGTGTCCCAGGGGGG
AAGCAGTACAGCATGGAGGGTGCCCCTGCTGCGCCCTTCCGGCCCTCGCAAGGCTTCCTGAGCCGACGGCTAAAAAG
CTCCATCAAACGAACGAAGTCACAACCCAAACTTGACCGGACCAGCAGCTTTCGCCAGATCCTGCCTCGCTTCCGAA
GTGCTGACCATGACCGGGCCCGGCTGATGCAAAGCTTTAAGGAGTCACACTCTCATGAGTCCTTGCTGAGTCCTAGC
AGTGCAGCTGAGGCATTGGAGCTCAACTTGGATGAAGATTCCATTATCAAGCCAGTGCACAGCTCCATCCTGGGCCA
GGAGTTCTGTTTTGAGGTAACAACTTCATCAGGAACAAAATGCTTTGCCTGTCGGTCTGCGGCCGAAAGAGACAAAT
GGATTGAGAATCTGCAGCGGGCAGTAAAGCCCAACAAGGACAACAGCCGCCGGGTAGACAATGTGCTAAAGCTGTGG
ATCATAGAGGCCCGGGAGCTGCCCCCCAAGAAGCGGTACTACTGTGAGCTCTGCCTGGATGACATGCTGTATGCACG
CACCACCTCCAAGCCCCGCTCTGCCTCTGGGGACACCGTCTTCTGGGGCGAGCACTTCGAGTTTAACAACCTGCCGG
CTGTCCGTGCCCTGCGGCTGCATCTGTACCGTGACTCAGACAAAAAGCGCAAGAAGGACAAGGCAGGCTATGTCGGC
CTGGTGACTGTGCCAGTGGCCACCCTGGCTGGGCGCCACTTCACAGAGCAGTGGTACCCTGTAACCCTGCCAACAGG
CAGTGGGGGATCTGGGGGCATGGGTTCGGGAGGGGGAGGGGGCTCGGGGGGTGGCTCAGGGGGCAAGGGCAAAGGAG
GTTGCCCGGCTGTGCGGCTGAAAGCACGTTACCAGACAATGAGCATCTTGCCCATGGAGCTATATAAAGAGTTTGCA
GAGTATGTCACCAACCATTATCGGATGCTGTGTGCAGTCTTGGAGCCCGCCCTGAATGTCAAAGGCAAGGAGGAGGT
TGCCAGTGCACTAGTTCACATCCTGCAGAGTACAGGCAAGGCCAAGGACTTCCTTTCAGACATGGCCATGTCTGAGG
TAGACCGGTTCATGGAACGGGAGCACCTCATATTCCGCGAGAACACGCTTGCCACTAAAGCCATAGAAGAGTATATG
AGACTGATTGGTCAGAAATACCTCAAGGATGCCATTGGAGAATTCATCCGTGCTCTGTATGAATCTGAGGAAAACTG
CGAGGTAGACCCTATCAAGTGCACAGCATCCAGTTTGGCAGAGCACCAGGCCAACCTGCGAATGTGCTGTGAGTTGG
CCCTGTGCAAGGTGGTCAACTCCCACTGCGTGTTCCCGAGGGAGCTGAAGGAGGTGTTTGCTTCGTGGCGGCTGCGC
TGCGCAGAGCGAGGCCGGGAGGACATCGCAGACAGGCTTATCAGCGCCTCACTCTTCCTGCGCTTCCTCTGCCCAGC
GATTATGTCGCCCAGTCTCTTTGGGCTTATGCAGGAGTACCCAGATGAGCAGACCTCACGAACCCTCACCCTCATTG
CCAAGGTCATCCAGAACCTGGCCAACTTTTCCAAGTTTACCTCAAAGGAGGACTTTCTGGGCTTCATGAATGAGTTT
CTGGAGCTGGAATGGGGTTCCATGCAGCAGTTTTTGTATGAGATCTCCAATCTGGACACGCTAACCAACAGCAGTAG
CTTTGAGGGTTACATCGACTTGGGCCGAGAGCTCTCCACACTGCATGCCCTACTCTGGGAGGTGCTGCCCCAGCTCA
GCAAGGAAGCCCTCCTGAAGCTGGGTCCACTGCCCCGGCTCCTCAACGACATCAGCACAGCTCTGAGGAACCCCAAC
ATCCAAAGGCAGCCAAGCCGCCAGAGTGAGCGGCCCCGGCCTCAGCCTGTGGTACTGCGGGGGCCATCGGCTGAGAT
GCAGGGCTACATGATGCGGGACCTCAACAGCTCTATGGACATGGCTCGCCTCCCCTCCCCAACCAAGGAAAAGCCAC
CCCCACCACCGCCTGGTGGTGGTAAAGACCTGTTCTATGTAAGCCGTCCACCCCTGGCCCGTTCCTCACCAGCATAC
TGCACGAGCAGCTCGGACATCACAGAGCCAGAGCAGAAGATGCTGAGTGTCAACAAGAGTGTGTCCATGCTGGACTT
ACAGGGTGATGGGCCTGGTGGCCGCCTCAACAGCAGCAGTGTTTCGAACCTGGCGGCCGTAGGGGACCTGCTGCACT
CAAGCCAGGCCTCGCTGACAGCAGCCTTGGGGCTACGGCCTGCGCCTGCCGGACGCCTCTCCCAGGGGAGTGGCTCA
TCCATCACGGCGGCTGGCATGCGCCTCAGCCAGATGGGTGTCACCACAGACGGTGTCCCTGCCCAGCAACTGCGAAT
CCCCCTCTCCTTCCAGAACCCTCTCTTCCACATGGCTGCTGATGGGCCAGGTCCCCCAGGCGGCCATGGAGGGGGCG
GTGGCCATGGCCCACCTTCCTCCCATCACCACCACCACCACCATCACCACCACCGAGGTGGAGAGCCCCCTGGGGAC
ACCTTTGCCCCATTCCATGGCTATAGCAAGAGTGAGGACCTCTCTTCCGGGGTCCCCAAGCCCCCTGCTGCCTCCAT
CCTTCATAGCCACAGCTACAGTGATGAGTTTGGACCCTCTGGCACTGACTTCACCCGTCGGCAGCTTTCACTCCAGG
ACAACCTGCAGCACATGCTGTCCCCTCCCCAGATCACCATTGGTCCCCAGAGGCCAGCCCCCTCAGGGCCTGGAGGT
GGGAGCGGTGGGGGCAGCGGTGGGGGTGGCGGGGGCCAGCCGCCTCCATTGCAGAGGGGCAAGTCTCAGCAGTTGAC
AGTCAGCGCAGCCCAGAAACCCCGGCCATCCAGCGGGAATCTATTGCAGTCCCCAGAGCCAAGTTATGGCCCCGCCC
GTCCACGGCAACAGAGCCTCAGCAAGGAGGGCAGCATTGGGGGCAGCGGGGGCAGCGGTGGCGGAGGGGGTGGGGGG
CTGAAGCCCTCCATCACCAAGCAGCATTCTCAGACACCATCCACATTGAACCCCACAATGCCAGCCTCTGAGCGGAC
AGTGGCCTGGGTCTCCAACATGCCTCACCTGTCGGCTGACATCGAGAGTGCCCACATCGAGCGGGAAGAGTACAAGC
TCAAGGAGTACTCAAAATCGATGGATGAGAGCCGGCTGGATAGGGAGTACGAGGAGGAGATTCACTCACTGAAAGAG
CGGCTGCACATGTCCAACCGGAAGCTGGAAGAGTATGAGCGGAGGCTGCTGTCCCAGGAAGAACAAACCAGCAAAAT
CCTGATGCAGTATCAGGCCCGACTGGAGCAGAGTGAGAAGAGGCTAAGGCAGCAGCAGGCAGAGAAGGATTCCCAGA
TCAAGAGCATCATTGGCAGGCTGATGCTGGTGGAGGAGGAGCTGCGCCGGGACCACCCCGCCATGGCTGAGCCGCTG
CCAGAACCCAAGAAGAGGCTGCTCGACGCTCAGAGAGGCAGCTTCCCCCCTTGGGTCCAACAAACCCGCGTGTGACG
CTGGCCCCACCGTGGAATGGCCTGGCCCCCCCAGCCCCACCACCCCCACCCCGGCTGCAGATTACGGAGAACGGCGA
GTTCCGAAACACCGCAGACCACTAGCCCACCCAGCATCAGAGACCTTCTCTTCCTTTCCTGTGCACCCCACCCTGTA
ACAGCACCAACCACCAGGATTGGACATCACCGAGGAACAGCGGGATTGCCTCCCCGAATGCCTCCCTGGGAGGCACA
CTGATTGCCCACCCCCACCACTGCACCATTTCCAGGAGGGAGAGTGGGGACCCTCAGCCGCCCCCTTTTCCTTCCCA
TTGGGGTGCTGCCCTCTCTTTGACCCCCAGGGACCCTTGCCCCAGGACACCGCCTACCCCGTACAGACCCCTTCACT
CCGGGGTGCTATCCCCATCCTCTGCCTCATCGTTCCCCTGAGCACTGGGGGACAGACCCTCACCCCCACCCTGGGGG
TGTGGCACCTCCAAACTTTCAACTTCAGGGTGATTTTTTTAGCAGTAACCAGAGCTGACAATCTAACTCCCCTCCAC
CGCCCCATTTTGGCCTCCCCTGCCCCCCTTGTTATGGGGAGGGGACCCCGGGTGAGGGGGCCCTATTACCCCTTGAT
TTCTCAGGAGCGTCTGGGGGGGCTCAGCACGCACAAACTCCTTCTCCTTCTACCACTCTTAAATTTACTCCCTCCCC
ACCCAGAACCCAGATGGGGTGGAGGGGGCCACCGGGGCAGGGAGGGGGCGGCAAGGGGGGAATGGGAGTTGTCTCCC
CTTCTCCCCACACCTGATCTGCTCTCGGCTGGTCCCAGAGCGGGGTGAGGGGGCTTATGCCCCCCCCTCCCCCAGTG
TGTTGGGTGGGGTGGAATTGAGGTTAGGGTGAGGGGTCAGGGTTTAGGAGGGTGTGTATGTTGGGAGGACAGGCTAG
TTGATCTGTCCTACTCTGACACACAGTCCCCTCTGCCCCTTCCTTCTCTCTTCTTGGTCTCTACTCCCAGGGGGAGG
GGGGAACTTACTCTAGGAAAAGCCATGTCTCTCTCCCCCAGGGTGGGGGGACCTGTGTTGGAGGAGGGGTGTTGGGG
GGCCCCCTTCCATGACTCTGTCCCCTGGGGGAGGTAGGACAGGGCTGGGCTTCCCTCTCATCCTCCCCCTCCCAATC
TCCTTCCACCTCCCTCCCTCCCGCCAGCTCCACGATTTTTCGGTGTTTCTCTGTACATAGTTTTCTGGCGGGATAGG
GGAGGTAGGATGGATGGGGTTTGGGGTGGGTAGGCCATGGGAGGGGAGAAGCCCCTCCTTGGCACCCCCTCTTCCCT
GACTGCTGTCCCCTACCCAGCCTTGCCCCCTTCATCCTTTTGCGTTTGGTATTGAGACTCTCCTAGACTCTACTCCT
CTTTCTTTTGTATGGACAGTTCCCCTTCAGTCCCATCCCCCTACACATACACCCAGCCGGGGCCAAATTTATACTTA
TATAAAAGTTGTAAATATGTGAAATTTTATCCCTGTGCCCTTTCCCCACCTCAGGCCCTACCCCTGGACCCTCCCCA
ACCTTCCTTCTCTCTTCTTTGGCTGTTGTAATTATCTGGGGTTTGTACTGTACATATCCGGGGTGTGTGTGTGTGGG
CTGGGGGCAACCCTTCTGTACAGAGCTTCCTGGCCCCCTCCCCCCCCGCCCCTCTGCTTCCCTCCCCACCCACCACC
TCAAGGGTAGGGAGTTGCTCTTCCTACCTGTTTTATTTTGTTTTCTCGTTCTCCCTCCCCACCCCACTCCCAGCCTT
ATCTATCCCCCCTCACTGTCCCCTTTTCTCCACTCCCAGCCCCATTTCCTTTTTTTCTGGAGTGTGTGGTGAAACAG
AAAAAAACATGTTTAATAAACGGAGATTGTTCTTTTA

Primers or probes can be designed so that they hybridize under stringent conditions to mutant nucleotide sequences of STXBP1, SYNGAP1, or SLC6A1, but not to the respective wild-type nucleotide sequences. Primers or probes can also be prepared that are complementary and specific for the wild-type nucleotide sequence of STXBP1, SYNGAP1, or SLC6A1, but not to any of the corresponding mutant nucleotide sequences. In some embodiments, the mutant nucleotide sequences of STXBP1, SYNGAP1, or SLC6A1 may be a frameshift mutation, a missense mutation, a deletion, an insertion, a nonsense mutation, an inversion, or a translocation, that results in the loss of expression and/or activity of STXBP1, SYNGAP1, or SLC6A1 (i.e., loss of function mutations).

In some embodiments, detection can occur through any of a variety of mobility dependent analytical techniques based on the differential rates of migration between different nucleic acid sequences. Exemplary mobility-dependent analysis techniques include electrophoresis, chromatography, mass spectroscopy, sedimentation, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described in Published PCT Applications WO04/46344 and WO01/92579. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003).

It is also understood that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. In some embodiments, unlabeled reaction products may be detected using mass spectrometry.

### NGS Platforms

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLID sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454™ GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added poly A tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLID™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

### Pharmaceutical Compositions Including Phenylbutyrate or Pharmaceutically Acceptable Salts Thereof

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Modes of Administration and Effective Dosages

Any method known to those in the art for contacting a cell, organ or tissue with one or more compositions including phenylbutyrate or a pharmaceutically acceptable salt thereof disclosed herein may be employed. Suitable methods include in vitro, ex vivo, or in vivo methods. In vivo methods typically include the administration of one or more compositions including phenylbutyrate or a pharmaceutically acceptable salt thereof to a mammal, suitably a human. When used in vivo for therapy, the one or more compositions including phenylbutyrate or a pharmaceutically acceptable salt thereof as described herein are administered to the subject in effective amounts (i.e., amounts that have desired therapeutic effect). The dose and dosage regimen will depend upon the degree of the disease state of the subject, the characteristics of phenylbutyrate or the particular pharmaceutically acceptable salt thereof used, e.g., its therapeutic index, and the subject's history.

The effective amount may be determined during pre-clinical trials and clinical trials by methods familiar to physicians and clinicians. An effective amount of one or more compositions including phenylbutyrate or a pharmaceutically acceptable salt thereof useful in the methods may be administered to a mammal in need thereof by any of a number of well-known methods for administering pharmaceutical compounds. The compositions including phenylbutyrate or a pharmaceutically acceptable salt thereof may be administered systemically or locally.

The one or more compositions including phenylbutyrate or a pharmaceutically acceptable salt thereof described herein can be incorporated into pharmaceutical compositions for administration, singly or in combination, to a subject for the treatment or prevention of a disease or condition described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more compositions including phenylbutyrate or a pharmaceutically acceptable salt thereof disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34 (7-8): 915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly a-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8): 915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4 (3): 201-9 (1994); and Gregoriadis, “Engineering Liposomes for Drug Delivery: Progress and Problems,” Trends Biotechnol., 13 (12): 527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more compositions including phenylbutyrate or a pharmaceutically acceptable salt thereof disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more compositions including phenylbutyrate (or a pharmaceutically acceptable salt thereof) concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more compositions including phenylbutyrate or a pharmaceutically acceptable salt thereof may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Therapeutic Methods of the Present Technology

In one aspect, the present disclosure provides a method for selecting a patient diagnosed with or at risk for STXBP1 encephalopathy (STXBP1-E) for treatment with a composition including phenylbutyrate or a pharmaceutically acceptable salt thereof comprising detecting the presence of a STXBP1 haploinsufficient allele in a biological sample obtained from the patient; and administering an effective amount of a composition including phenylbutyrate or a pharmaceutically acceptable salt thereof to the patient. In another aspect, the present disclosure provides a method for treating STXBP1 encephalopathy (STXBP1-E) in a patient in need thereof comprising administering an effective amount of a composition including phenylbutyrate or a pharmaceutically acceptable salt thereof to the patient, wherein the patient comprises a STXBP1 haploinsufficient allele.

In one aspect, the present disclosure provides a method for selecting a patient diagnosed with or at risk for SYNGAP1 encephalopathy for treatment with a composition including phenylbutyrate or a pharmaceutically acceptable salt thereof comprising detecting the presence of a SYNGAP1 haploinsufficient allele in a biological sample obtained from the patient; and administering an effective amount of a composition including phenylbutyrate or a pharmaceutically acceptable salt thereof to the patient. In another aspect, the present disclosure provides a method for treating SYNGAP1 encephalopathy in a patient in need thereof comprising administering an effective amount of a composition including phenylbutyrate or a pharmaceutically acceptable salt thereof to the patient, wherein the patient comprises a SYNGAP1 haploinsufficient allele.

The STXBP1 or SYNGAP1 haploinsufficient allele may comprise a nonsense mutation, a missense mutation, a deletion, an inversion or a frameshift mutation. In certain embodiments, the STXBP1 haploinsufficient allele is S311FfsX3, R292P, or R190W. In some embodiments, the SYNGAP1 haploinsufficient allele is R579X, K1185*, R621*, Y928*, or C576*. Additionally or alternatively, in some embodiments, the STXBP1 or SYNGAP1 haploinsufficient allele is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). In any and all of the preceding embodiments, the composition comprises glycerol phenylbutyrate.

Additionally or alternatively, in certain embodiments, the patient does not comprise a dominant-negative STXBP1 mutation, optionally wherein the dominant-negative STXBP1 mutation is selected from the group consisting of V84D, C180Y, L183R, P335L, R406C, R406H, M443R, P480L, G544D, G544V, R551C, C552R, and T574R. In some embodiments, the patient does not comprise one or more STXBP1 mutations selected from among P139L, E283K, and Q229*. In some embodiments, the patient does not comprise one or more SYNGAP1 mutations selected from among S190Cysfs*2 or S225HfsX26. In some embodiments, the patient does not comprise an IRF2BPL mutation comprising A101ThrfsTer18.

In any of the preceding embodiments of the methods disclosed herein, the STXBP1-E presents as infantile spasms, epilepsy of infancy migrating focal seizure, Dravet syndrome or non-syndromic epilepsy. Additionally or alternatively, in certain embodiments, the patient does not exhibit active epileptic spasms. In any and all embodiments of the methods disclosed herein, the patient exhibits one or more of autism, low tone, movement disorders (including ataxia or bruxism), abnormal EEGs (>60% with focal or multifocal epileptiform discharges), or abnormal MRI brain imaging (including atrophy, thin corpus callosum, or delayed myelination).

In one aspect, the present disclosure provides a method for treating developmental and epileptic encephalopathy (DEE) in a patient in need thereof comprising administering an effective amount of a composition including phenylbutyrate or a pharmaceutically acceptable salt thereof to the patient, wherein the patient does not exhibit active epileptic spasms. The patient may comprise a STXBP1 haploinsufficient allele, a SYNGAP1 haploinsufficient allele, and/or a SLC6A1 haploinsufficient allele. Additionally or alternatively, in some embodiments, the STXBP1 haploinsufficient allele, SYNGAP1 haploinsufficient allele, or SLC6A1 haploinsufficient allele comprises a nonsense mutation, a missense mutation, a deletion, an inversion or a frameshift mutation. Examples of STXBP1 haploinsufficient alleles include, but are not limited to, S311FfsX3, R292P, or R190W. Examples of SYNGAP1 haploinsufficient allelles include, but are not limited to, R579X, K1185*, R621*, Y928Ter, or C576*. Examples of SLC6A1 haploinsufficient alleles include, but are not limited to D52N, L408WfsX26, A288V, G297R, A305T, V125M, S295L, G362R, D410E, L460R and W495X.

Additionally or alternatively, in certain embodiments, the patient does not comprise one or more STXBP1 mutations selected from the group consisting of V84D, C180Y, L183R, P335L, R406C, R406H, M443R, P480L, G544D, G544V, R551C, C552R, T574R, P139L, E283K, and Q229*. In some embodiments, the patient does not comprise one or more SYNGAP1 mutations selected from among S190Cysfs*2 or S225HfsX26. In some embodiments, the patient does not comprise an IRF2BPL mutation comprising A101ThrfsTer18. In any and all of the preceding embodiments, the composition comprises glycerol phenylbutyrate.

In any and all embodiments of the methods disclosed herein, the patient is an infant, a child or an adolescent. Additionally or alternatively, in some embodiments of the methods disclosed herein, the effective amount of the composition including phenylbutyrate or the pharmaceutically acceptable salt thereof is about 5 g/m²/day to about 12.5 g/m²/day. In some embodiments, the effective amount of the composition including phenylbutyrate or the pharmaceutically acceptable salt thereof is about 5.0 g/m²/day, about 5.1 g/m²/day, about 5.2 g/m²/day, about 5.3 g/m²/day, about 5.4 g/m²/day, about 5.5 g/m²/day, about 5.6 g/m²/day, about 5.7 g/m²/day, about 5.8 g/m²/day, about 5.9 g/m²/day, about 6.0 g/m²/day, about 6.1 g/m²/day, about 6.2 g/m²/day, about 6.3 g/m²/day, about 6.4 g/m²/day, about 6.5 g/m²/day, about 6.6 g/m²/day, about 6.7 g/m²/day, about 6.8 g/m²/day, about 6.9 g/m²/day, about 7.0 g/m²/day, about 7.1 g/m²/day, about 7.2 g/m²/day, about 7.3 g/m²/day, about 7.4 g/m²/day, about 7.5 g/m²/day, about 7.6 g/m²/day, about 7.7 g/m²/day, about 7.8 g/m²/day, about 7.9 g/m²/day, about 8.0 g/m²/day, about 8.1 g/m²/day, about 8.2 g/m²/day, about 8.3 g/m²/day, about 8.4 g/m²/day, about 8.5 g/m²/day, about 8.6 g/m²/day, about 8.7 g/m²/day, about 8.8 g/m²/day, about 8.9 g/m²/day, about 9.0 g/m²/day, about 9.1 g/m²/day, about 9.2 g/m²/day, about 9.3 g/m²/day, about 9.4 g/m²/day, about 9.5 g/m²/day, about 9.6 g/m²/day, about 9.7 g/m²/day, about 9.8 g/m²/day, about 9.9 g/m²/day, about 10.0 g/m²/day, about 10.1 g/m²/day, about 10.2 g/m²/day, about 10.3 g/m²/day, about 10.4 g/m²/day, about 10.5 g/m²/day, about 10.6 g/m²/day, about 10.7 g/m²/day, about 10.8 g/m²/day, about 10.9 g/m²/day, about 11.0 g/m²/day, about 11.1 g/m²/day, about 11.2 g/m²/day, about 11.3 g/m²/day, about 11.4 g/m²/day, about 11.5 g/m²/day, about 11.6 g/m²/day, about 11.7 g/m²/day, about 11.8 g/m²/day, about 11.9 g/m²/day, about 12.0 g/m²/day, about 12.1 g/m²/day, about 12.2 g/m²/day, about 12.3 g/m²/day, about 12.4 g/m²/day, or about 11.5 g/m²/day.

For therapeutic and/or prophylactic applications, a composition comprising phenylbutyrate or a pharmaceutically acceptable salt thereof is administered to the subject. In some embodiments, the composition is administered one, two, three, four, or five times per day. In some embodiments, the composition is administered more than five times per day. Additionally or alternatively, in some embodiments, the composition is administered every day, every other day, every third day, every fourth day, every fifth day, or every sixth day. In some embodiments, the composition is administered weekly, bi-weekly, tri-weekly, or monthly. In some embodiments, the composition is administered for a period of one, two, three, four, or five weeks. In some embodiments, the composition is administered for six weeks or more. In some embodiments, the composition is administered for twelve weeks or more. In some embodiments, the composition is administered for a period of less than one year. In some embodiments, the composition is administered for a period of more than one year. In some embodiments, the composition is administered throughout the subject's life.

In some embodiments of the methods of the present technology, the composition is administered daily for 1 week or more. In some embodiments of the methods of the present technology, the composition is administered daily for 2 weeks or more. In some embodiments of the methods of the present technology, the composition is administered daily for 3 weeks or more. In some embodiments of the methods of the present technology, the composition is administered daily for 4 weeks or more. In some embodiments of the methods of the present technology, the composition is administered daily for 6 weeks or more. In some embodiments of the methods of the present technology, the composition is administered daily for 12 weeks or more. In some embodiments, the composition is administered throughout the subject's life.

In any of the preceding embodiments of the methods disclosed herein, the composition including phenylbutyrate or the pharmaceutically acceptable salt thereof is administered enterally, optionally wherein the composition is administered via a nasogastric or gastrostomy tube. Additionally or alternatively, in some embodiments of the methods disclosed herein, the composition is administered daily for at least 6 weeks, at least 8 weeks, at least 10 weeks, at least 12 weeks or at least 14 weeks, optionally wherein the composition is administered thrice a day.

In any and all embodiments of the methods disclosed herein, administration of the composition results in amelioration of one or more of reduction in seizures, improvement in cognition, reduction in abnormal movement, improvement in sleep quality, improved gait, improved quality life, improved mood, reduction in bruxism, improvement in gross motor skills, improvement in fine motor skills, improvement in communication skills, or reduction in family burden.

Additionally or alternatively, the methods disclosed herein further comprise administering to the patient one or more of steroids, vigabatrin, or a ketogenic diet.

### EXAMPLES

The present technology is further illustrated by the following Examples, which should not be construed as limiting in any way. The examples herein are provided to illustrate advantages of the present technology and to further assist a person of ordinary skill in the art with preparing or using the methods of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects, or embodiments of the present technology.

### Example 1: Study of Glycerol Phenylbutyrate

Glycerol phenylbutyrate (trade name “Ravicti”) is an FDA approved medication for adjunctive therapy in children and adults with urea cycle disorders, and provides clinical benefit for cholestasis and thalassemia. It crosses the blood-brain barrier in mice and non-human primates (via the monocarboxylate transporter 1 mechanism) and has been studied for human neurological conditions such as spinal muscular atrophy, brain tumors (NCT00006450), and Huntington's disease (also NCT00212316). In addition, animal data suggest phenylbutyrate may be therapeutic for dementia, Parkinson's disease, and ischemic stroke.

For the approved indication of urea cycle disorders, glycerol phenylbutyrate is hydrolyzed to phenylbutyrate (PBA) through pancreatic lipases. Phenylbutyrate is then metabolized into the major metabolite phenylacetate (PAA), which is the active moiety that conjugates with glutamine via acetylation to form phenylacetylglutamine (PAGN) in the liver and kidneys, which is excreted by the kidneys. This helps to reduce the levels of ammonia in patients with urea cycle disorders.

The mechanism of action of phenylbutyrate in the treatment of STXBP1-E, based on the preclinical research, is expected to be mediated by PBA. Since patients with STXBP1-E do not have a urea cycle disorder, PAA may deplete glutamate levels, resulting in a low level of PAGN and higher levels of PAA.

Glycerol phenylbutyrate has an established side effect profile. Based on Lexicomp, the side effects with reported frequency include headache (14%), diarrhea (16%), flatulence (14%), fatigue (7%), abdominal pain (7%), decreased appetite (7%), vomiting (7%), dyspepsia (5%), and nausea (2%). Additional side effects with frequency not well defined include dizziness, peripheral neuropathy, seizure, and tremor. Side effects with less than 1% or from postmarketing surveillance and case reports include body odor, burning sensation in mouth, dysgeusia, gagging, and retching. Other side effects that were actively monitored for, based on review of other literature, included amenorrhea/menstrual dysfunction, decreased appetite, constipation, rectal bleeding, pancreatitis, aplastic anemia, arrhythmia, renal tubular acidosis, and rash. In addition, phenylbutyrate may reduce the effectiveness of midazolam, which is often used as an anti-seizure medication.

### Methods

### Participants

After institutional review board approval, 20 participants were enrolled in a single treatment group, multiple-dose, open-label, pilot study to evaluate the safety, tolerability, pharmacokinetics (PK) and pharmacodynamics (PD) of glycerol phenylbutyrate in children with STXBP1-E and SLC6A1-NDD.

Participants in the study were patients aged 2 months to 17 years with STXBP1-E and seizures or with SLC6A1-NDD. People with SLC6A1 typically experience seizures later in life (typically middle of 1st decade) and so seizures were not an entry criterion for participants with SLC6A1. During enrollment preference was given to patients between 2 months and 3 years old (target of 5 patients) and patients between 4 and 10 years old (target 5 patients). This is because younger patients up to 3 years old may be more sensitive to possible pharmacodynamics and efficacy endpoints and, to a lesser extent, the same may be true for patients 4 to 10 years, as compared to older patients. The study enrolled 11 patients, including 4 children between 2 months and 2 years.

The enrollment period and the study duration were February 2021 through the end of January 2023. Two facilities enrolled participants, Weill Cornell Medicine and Children's Hospital Colorado.

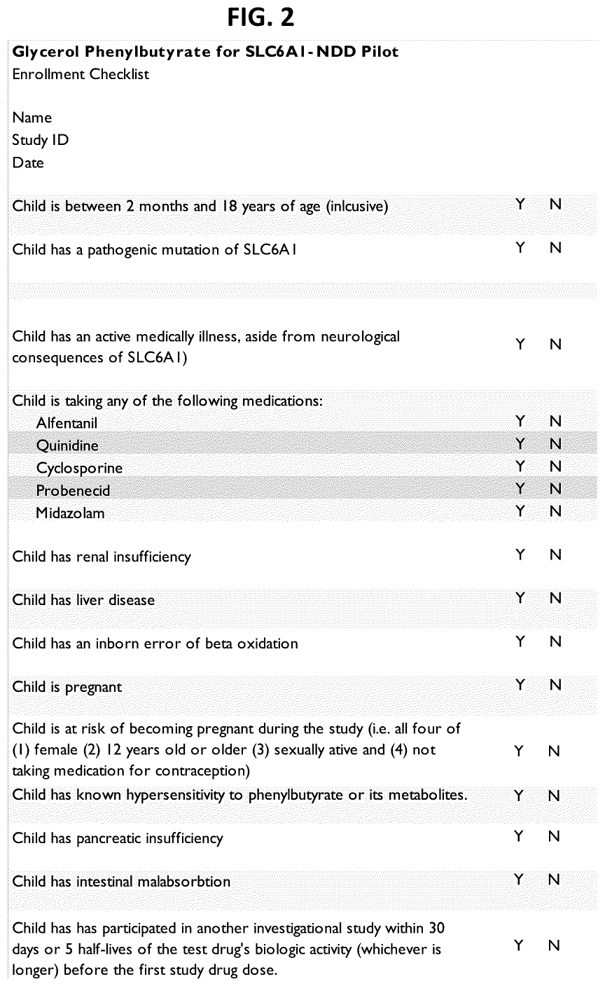

FIG. 2 is an enrollment checklist for enrollment in this study. The participants were eligible for the study if they satisfied all of the following criteria at the time of screening for enrollment. The participants must have been diagnosed with STXBP1-E or SLC6A1-NDD; confirmed by laboratory report (i.e., a genetic test with a pathogenic or likely pathogenic mutation of STXBP1 or SLC6A1 and a clinical picture consistent with the disorder, as determined by the Investigator). Children with the appropriate clinical picture, a de novo variant of uncertain significance in STXBP1 or SLC6A1 were also eligible for enrollment, at the discretion of the Investigator. If a patient's mutation was classified as definitively non-pathogenic, the patient was not enrolled. “Appropriate clinical picture” was at the discretion of the Investigator. The participants were between 2 months and 17 years of age, inclusive. For children with STXBP1-E, the child must have had at least one seizure in the past 30 days prior to enrollment to be eligible. The participants were in general good health, aside from neurological consequences of STXBP1-E or SLC6A1-NDD, as determined by having no concurrent medical illness that, in the opinion of the site investigator, places the participant at increased risk of adverse drug reactions or will interfere with study follow-up. The participants had normal laboratory test results (≤1.5×upper limit of normal [ULN]) for serum aminotransferase (aspartate aminotransferase [AST] and alanine aminotransferase [ALT]) concentrations and ammonia. The participants had normal renal function, with estimated glomerular filtration rate >90 mL/minute/1.73 m² at screening (using the Chronic Kidney Disease Epidemiology Collaboration equation). The participants had a platelet count >150×103/μL at screening. The participants had a QT interval corrected with Fridericia's formula (QTcF)<450 msec on the Screening EKG. The participants had a parent or guardian that was able to comprehend and willing to sign an informed consent form (ICF).

Subjects were not considered eligible to participate in this study if any one of the following exclusion criteria was satisfied at screening (and prior to dosing). Subjects were not eligible if they had participated in another investigational study within 30 days or 5 half-lives of the test drug's biologic activity (whichever is longer) before the first study drug dose. Subjects were not eligible if they had a QT interval corrected with Fridericia's formula (QTcF)≥450 msec on the Screening ECG. Subjects were not eligible if they had an active medical illness that would preclude participation in the study (as determined by the Investigator). Subjects were not eligible if they had a clinical laboratory evaluation outside of the laboratory reference range, unless deemed not clinically significant by the Investigator and the Sponsor. Subjects were not eligible if they were unable to comply with the study protocol. Subjects were not eligible if they had poor venous access and/or could not tolerate venipuncture. Subjects were not eligible if they were pregnant. Subjects were not eligible if they were a female of child-bearing age (12 years old or older) and were known to be sexually active, and not taking medication for contraception. Subjects were not eligible if they had a known hypersensitivity to phenylbutyrate. Signs of hypersensitivity include wheezing, dyspnea, coughing, hypotension, flushing, nausea, and rash. Subjects were not eligible if they were taking alfentanil, quinidine, cyclosporine, or probenecid, which are known to interact with phenylbutyrate. For subjects who had taken any of these medications in the past, the last dose must have been taken at least 1 week prior to enrollment into the study. Subjects were not eligible if they had inborn errors of beta oxidation. Subjects were not eligible if they had pancreatic insufficiency or intestinal malabsorption.

### Medication and Dosing

The study agent, glycerol phenylbutyrate (trade name “Ravicti”) is an FDA approved medication used for urea cycle disorders in children and adults.

Ravicti is a clear, colorless to pale yellow liquid for oral administration. It is a prodrug of phenylbutyrate, the active ingredient in NaPBA (sodium phenylbutyrate), and a pre-prodrug of the metabolite phenylacetate. The Ravicti drug product is an undiluted and unformulated glycerol phenylbutyrate drug substance (i.e., there are no preservatives or inactive ingredients) filled into glass bottles capped under nitrogen. It is a nearly odorless and tasteless liquid that is colorless to pale yellow at room temperature. glycerol phenylbutyrate is intended for oral administration only.

A daily dose of 11.2 mL/m², equivalent to 12.4 g/m², was given, enterally (by mouth or, if necessary, by nasogastric or gastrostomy tube). The daily dose was divided into three equally divided doses, with each dose given with food.

The size of the dose was calculated using the participant's body surface area. The participant's body surface area was calculated according to the following equation:

$$BSA = \frac{\sqrt{height ( cm ) \times weight ( kg )}}{3600}$$

For example, a participant weighing 30 kg and having a height of 100 cm had a daily dose of about 10 mL/day (3.5 mL tid). Doses were rounded up to the nearest 0.5 mL. The maximum daily dose was 17.5 mL/day (19 g/day) of glycerol phenylbutyrate. In case of dosing delays or irregularities, caregivers were instructed to modify the dosing schedule on that day such that the participant received the entire daily dose. Daily doses of the study agent were administered for 6 weeks, followed by a 2-week taper. The dosing was consistent with the dosing guidelines in the FDA approved Medication Guide (www.accessdata.fda.gov/drugsatfda_docs/label/2017/203284s0051bl.pdf).

Dosing was sufficient to lead to therapeutic concentrations in the range of 0.25 mM to 1 mM in serum and CSF, based on the following observations. Doses of 3 g/m²/day of glycerol phenylbutyrate led to blood levels of phenylbutyric acid of 38 μg/mL (0.23 mM) in adults, suggesting that doses of 12 g/m²/day provided approximately concentrations of 1 mM in serum. CSF levels are approximately ½ to ¼ of serum levels, based on data from non-human primates, thus CSF levels of 0.25 to 0.5 mM were assumed.

For the first four days, participants were given a lower daily dose in order to assess for unanticipated adverse effects. On days 1 and 2, 1/9 of the full dose was administered; and on days 3 and 4, ⅓ of the full dose was administered. If an individual tolerated the medication at the lower doses but not higher doses, the daily dose may have been lowered for that individual.

### Study Objectives and Endpoints

The primary objective of the study was to evaluate the safety and tolerability of multiple doses of glycerol phenylbutyrate in patients with STXBP1-E and SLC6A1-NDD. The secondary objective of the study was to assess the pharmacokinetics of glycerol phenylbutyrate in patients with STXBP1-E and SLC6A1-NDD using peak plasma levels of phenylbutyrate. The exploratory objective of the study was to evaluate the pharmacodynamics of glycerol phenylbutyrate in patients with STXBP1-E and SLC6A1-NDD.

The primary endpoints for this pilot study were (1) safety and (2) tolerability. The safety endpoint was qualitative and included a description of the incidence, frequency, and severity of adverse events (including known side effects of Ravicti, changes in vital signs, EKG changes, EEG changes, increase in seizures, changes in clinical laboratory results, and/or changes in physical examination). The tolerability endpoint was quantitative and was measured for medication compliance (i.e. what percentage of the doses were taken).

The secondary endpoints were measuring plasma concentration of phenylbutyrate and active metabolites. Plasma levels of phenylbutyrate peaked at 2 hours after an oral dose. Thus investigators aimed to obtain levels within 90-150 minutes after the oral dose.

The exploratory endpoints were measuring the overall effect of glycerol phenylbutyrate on the following outcomes. The goal was to explore effects and roughly estimate effect sizes, without an expectation for achieving statistical significance. The outcomes measured were seizure burden, detailed description of EEG changes including quantitative analyses, qualitative description of abnormal movement captured on video, quality of life, development, movement disorder, behavior, sleep, drug compliance (adherence), and caregiver qualitative experience.

### Study Design

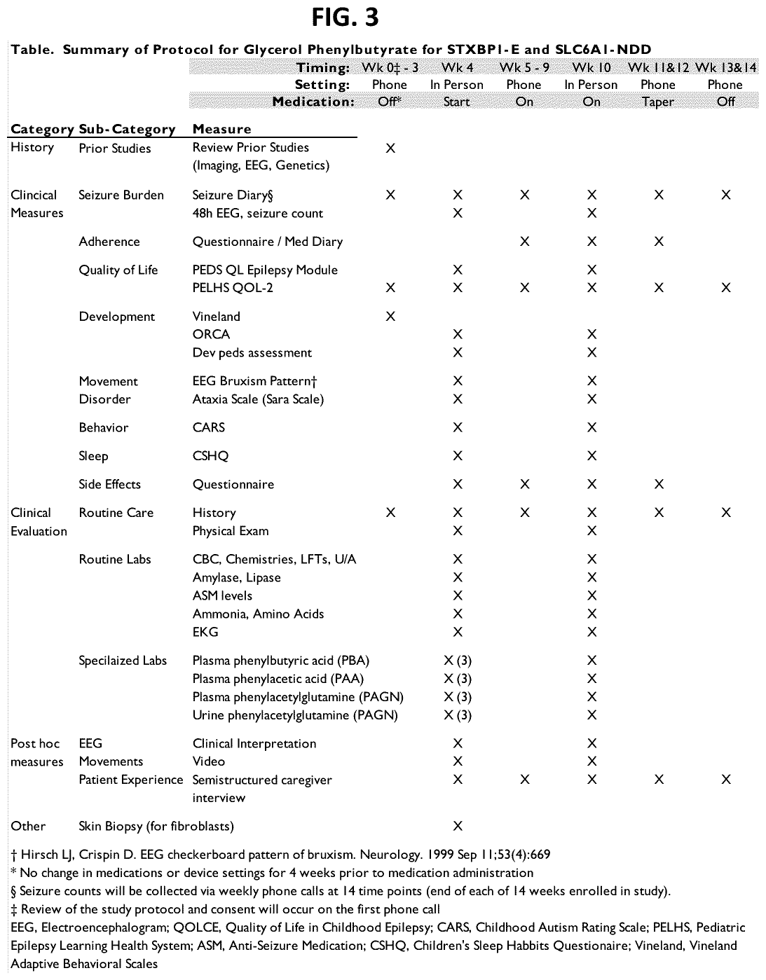

FIG. 3 is a schedule of assessments for measuring the effectiveness of glycerol phenylbutyrate to treat patients with STXBP1-E and SLC6A1-NDD. Each participant was enrolled for 14 weeks (4 weeks baseline, 8 weeks of drug exposure, and 2 weeks follow up). Participants were enrolled and followed for 4 weeks before using the medication to understand baseline characteristics (i.e. seizure frequency). The participants were then admitted for a 3-day inpatient admission for the first administration of glycerol phenylbutyrate and a battery of assessments, including video EEG. The participants were then followed for six weeks via weekly virtual visits. The participants were then admitted a second time for an overnight video EEG and additional assessments. Participants were then weaned of glycerol phenylbutyrate over two weeks and followed for two additional weeks.

If after clinical assessment by the investigator there was deemed to be clinical benefit outweighing clinical risk to the study participant, and there was a request for continued study participation by the child's caregiver, study participation may have continued for an additional 12 months.

Examples of clinical benefit include (but are not limited to) the following: reduction in seizures, improvement in cognition, reduction in abnormal movement, improvement in sleep quality, improved gait, improved quality life, improved mood, reduction in bruxism, improvement in gross motor skills, improvement in fine motor skills, improvement in communication skills, or reduction in family burden. For some children, “no clinical worsening” is an example of a clinical benefit, as the disease entities under study are often progressive.

### Study Assessments

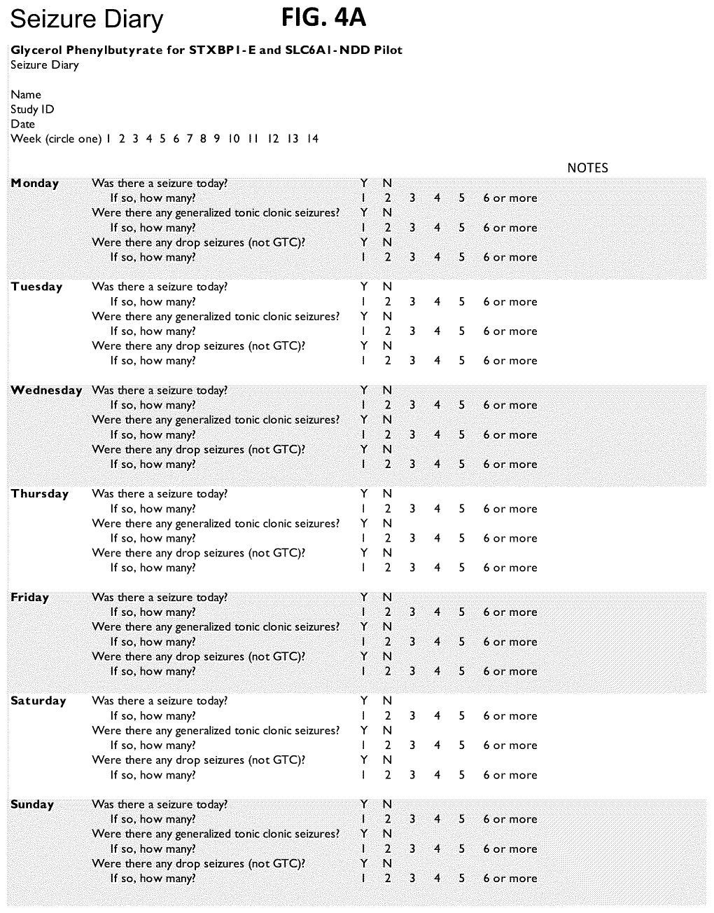

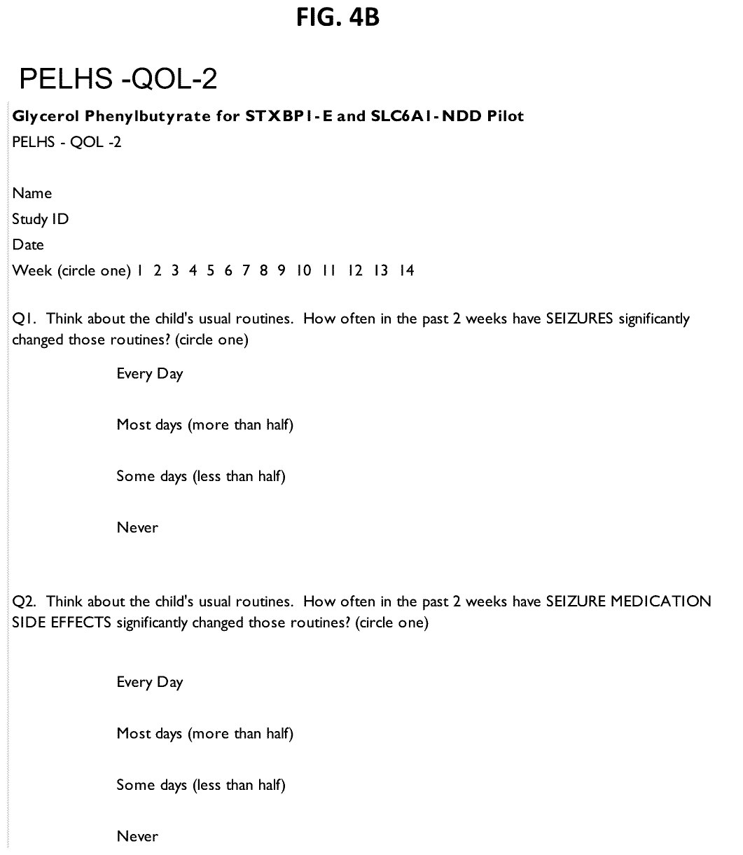

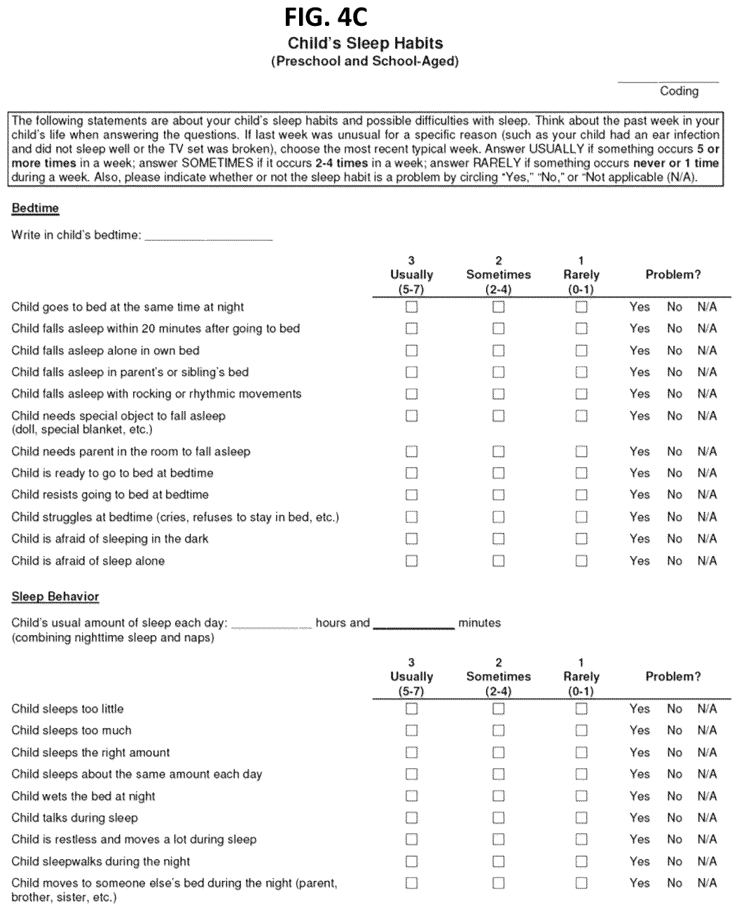

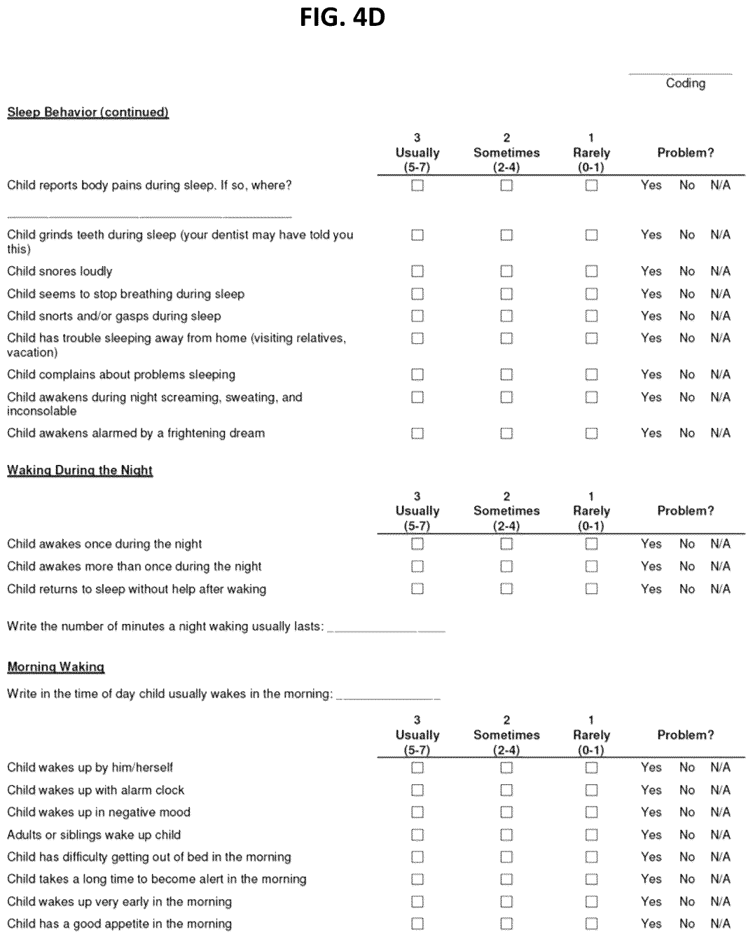

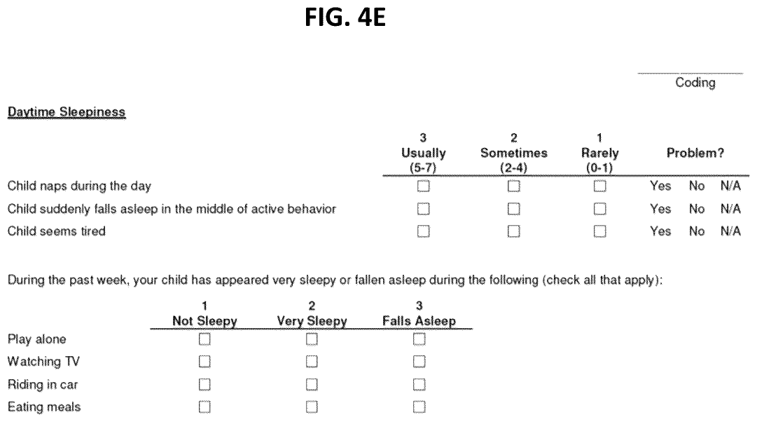

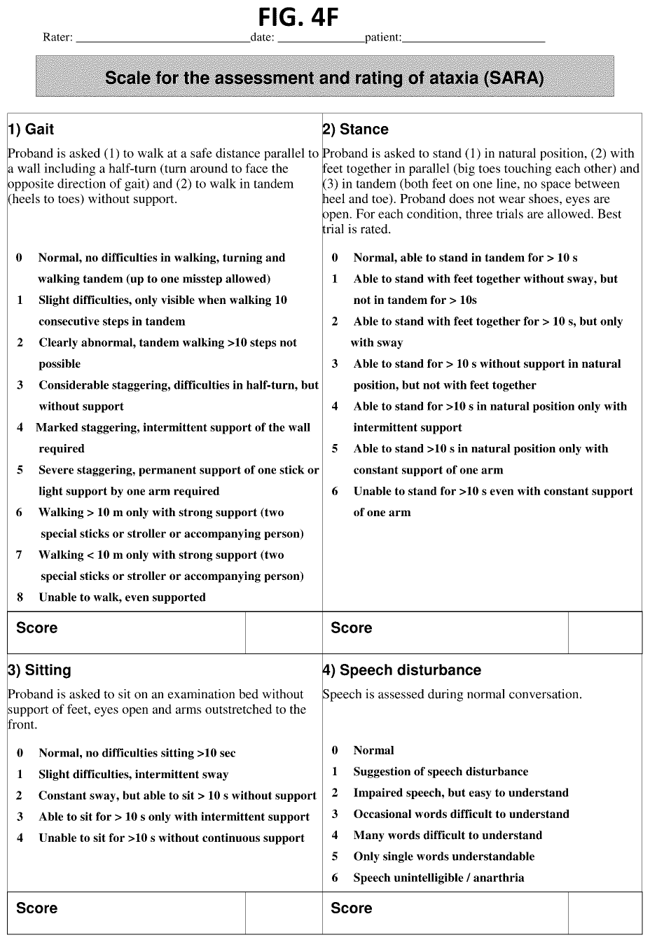

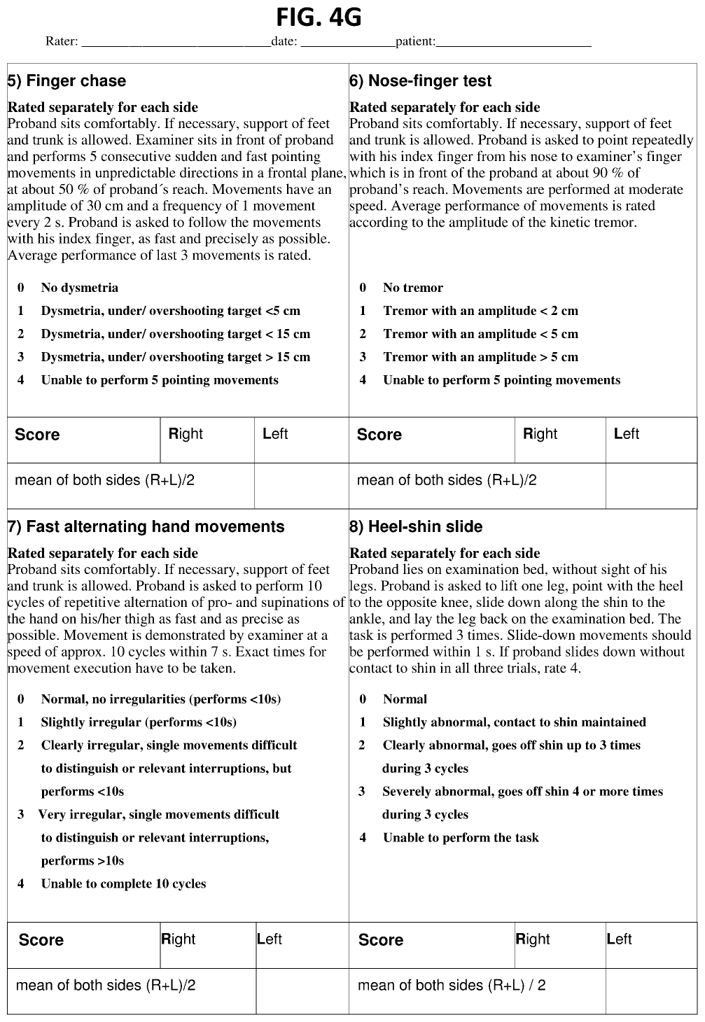

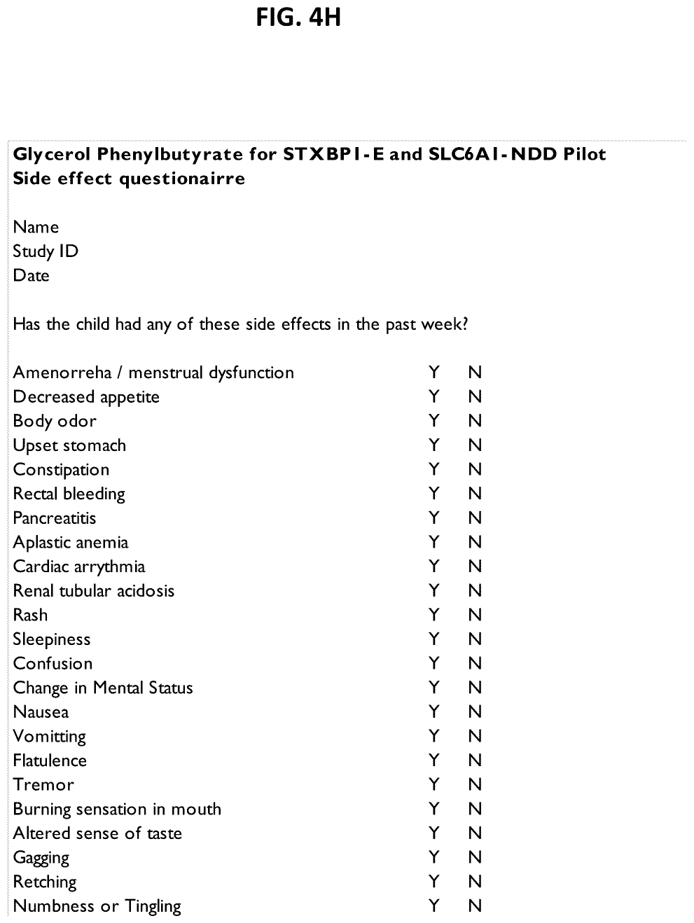

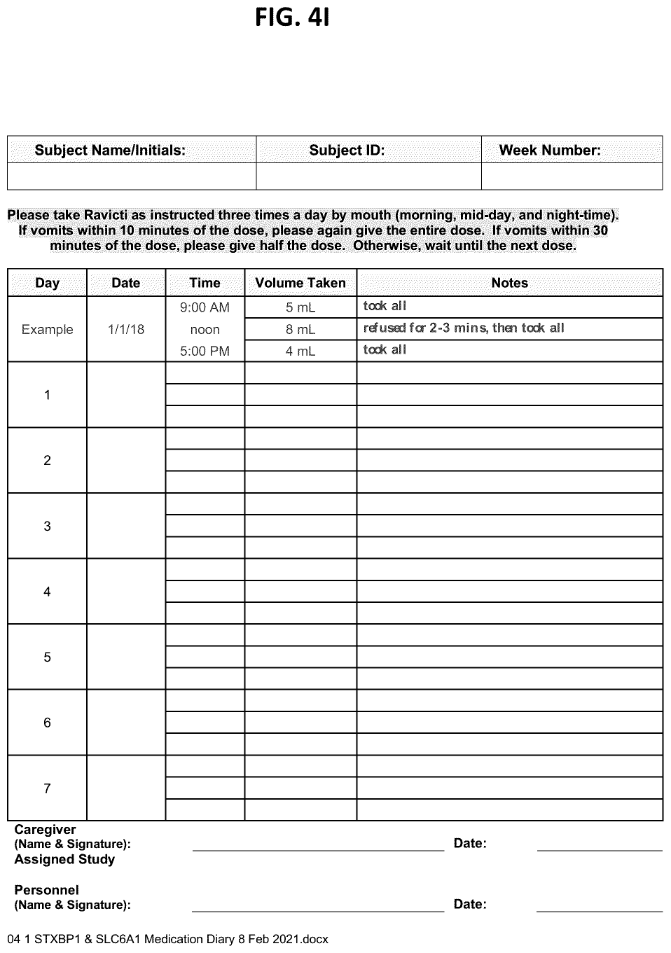

FIG. 4A is a seizure diary used by participants or their caregivers to self-report seizure incidence during the study.

Investigators noted EEG changes, including standard clinical interpretation of the EEG and video (particularly for abnormal movements).

Quality of life was assessed using the Pediatric Quality of Life Inventory (PEDSQL) epilepsy module, which is a validated scale about quality of life for children with epilepsy, and the Pediatric Epilepsy Learning Healthcare System Quality of Life survey (PELHS QOL 2), which is a set of two questions that ask how seizures and medication side effects alter usual routines. The PEDSQL is provided at eprovide.mapi-trust.org/instruments/pediatric-quality-of-life-inventory-epilepsy-module. FIG. 4B shows the PELHS-QOL-2 questionnaire.

Development was assessed using several pre-specified measures (Vineland, ORCA) as well as an assessment by a pediatric development expert (i.e. a physician, neuropsychologist, or psychologist with expertise in administering and interpreting developmental assessments). During the assessment, several additional validated measures may have been used. These may have included (but were not limited to) the Bayley Scales of Infant and Toddler Development, the Childhood Autism Rating Scale, the Child Behavior Checklist, the Behavior Rating Inventory of Executive Function, the Infant Toddler Social Emotional Assessment, the DAYC (Developmental Assessment of Young Children), or other similar validated scales.

Sleep was assessed using the Child's Sleep Habits Questionnaire (CHSQ), shown in FIGS. 4C to 4E.

Movement disorder was assessed by noting the presence or absence of bruxism in EEG data, and using the Scale for Assessment and Rating of Ataxia (SARA), which is a validated and reliable measure of ataxia, shown in FIGS. 4F-4G.

Behavior was assessed using the childhood autism rating scale (CARS), which is a validated screening measure for behaviors associated with autism. The CARS is a proprietary scale purchased from the distributor.

FIG. 4H is a side effect questionnaire that was used to assess side effects of taking the medication.

Drug compliance and adherence was assessed using parental reporting of how often the child was taking the study drug as prescribed. FIG. 4I shows the medication diary used for this assessment.

Caregiver qualitative experience was assessed by conducting 10- to 20-minute unstructured interviews with the family to understand their experiences with STXBP1-E and SLC6A1-NDD and the study drug.

### Study Procedures

FIG. 3 is a schedule of assessments for measuring the effectiveness of glycerol phenylbutyrate to treat patients with STXBP1-E and SLC6A1-NDD. Each participant had a series of visits with a study investigator over 14 weeks.

Visit 1 was a screening visit 4 weeks before the start of treatment with the medication. Visit 1 was conducted via phone or video. During the visit, the investigator reviewed with the participant prior studies (e.g., imaging, EEG, genetics), discussed keeping the seizure diary (which was started on the day of visit 1 and continued for all 14 weeks), administered the PELHS QOL-2, reviewed the participant's medical and surgical history, and instructed the participant's parent on completing the Vineland Adaptive Behavior Scales online no later than visit 4.

Visits 2-4 were a series of pre-initiation check-ins conducted approximately weekly starting the week after visit 1. During these visits, the investigator reviewed the participant's seizure diary, administered the PELHS QOL-2 to the participant, and reviewed the participant's medical history to confirm there were no changes in medication or device settings.

Visit 5 was a pre-treatment baseline visit conducted at least 28 days after visit 1 and the day before treatment initiation. This visit was conducted in person while the patient was admitted to hospital. During this visit, the investigator reviewed the participant's seizure diary and medical history. The investigator administered several assessments, including the PEDSQL Epilepsy Module, the PELHS QOL 2, the SARA Scale, the CARS, the CSHQ, and the ORCA. The participant was given a physical exam and had urine and blood samples collected for a series of laboratory tests. Laboratory tests included routine laboratory tests and specialized laboratory tests. Routine laboratory tests that were conducted included a complete blood count (CBC), chemistry panels, liver function tests (LFTs), urinalysis, amylase test, lipase test, anti-seizure medication (ASM) levels test, and electrocardiogram (EKG). Specialized laboratory tests that were conducted included plasma phenylbutyric acid test, plasma phenylacetic acid test, plasma phenylacetylglutamine test, and urine phenylacetylglutamine test. The investigator also captured a video of the participant's movements and took a skin punch biopsy from the participant. The investigator started a 48-hour long EEG of the participant. EEG data were collected using a full montage of EEG leads and the 10-20 system, and data was stored in the XLTEK digital EEG system.

During visit 5, a “substantial worsening” of seizures definition was calculated for each patient. “Substantial worsening” was defined as follows. Let x be the mean number of seizures per week in the four week observation period. A “substantial worsening” is more than x+3*sqrt (x) per week over a three week period.

During visit 5, each participant was evaluated by a developmental pediatrician, neuropsychologist, or psychologist. The evaluation included any of several validated developmental scales, such as the Bayley Scales of Infant and Toddler Development, the Childhood Autism Rating Scale, the Child Behavior Checklist, the Behavior Rating Inventory of Executive Function, the Infant Toddler Social Emotional Assessment, the DAYC (Developmental Assessment of Young Children), or other similar validated scales.

During visit 6, which was on the day after visit 5, the participant started taking the medication in the morning while still admitted to hospital. During the visit, the investigator reviewed the video of the EEG, noting the number of seizures, bruxism patterns, and clinical interpretation. The investigator also reviewed the participant's seizure diary and medical and surgical history, concomitant medications, and clinical assessment. The investigator captured another video of the participant's movements on the evening of the first day of medication administration after the day's doses were administered. Also that evening, blood and urine were collected from the participant for the specialized laboratory tests described above.

Visit 7 was the day after visit 6 and was also conducted in person. During visit 7, the investigator reviewed the video of the EEG, noting the number of seizures, bruxism patterns, and clinical interpretation. The investigator also reviewed the participant's seizure diary and conducted a clinical assessment. The participant's blood and urine were collected for routine and specialized laboratory tests. The investigator administered the side effects questionnaire and captured another video of the participant's movements.

Visits 8-13 were maintenance visits conducted approximately 0, 1, 2, 3, 4, and 5 weeks after treatment initiation, respectively. These visits were conducted by phone or video. During the visits, the investigator reviewed the participant's seizure diary, medical and surgical history, concomitant medications, and clinical assessment. The investigator administered the adherence questionnaire/medication diary, the PELHS QOL-2, and the side effects questionnaire. The investigator conducted an unstructured interview with the caregiver of the participant.

Visit 14 was a treatment evaluation visit and was conducted in person at least 40 days after the first day of medication administration. During the visits, the investigator reviewed the participant's seizure diary, medical and surgical history, concomitant medications, and clinical assessment. The investigator administered the PEDSQL Epilepsy Module, the PELHS QOL-2, the SARA Scale, the CSHQ, the Side Effects Questionnaire, the Adherence Questionnaire/Medication Diary, and the ORCA. The participant's blood and urine were collected for routine and specialized laboratory tests. The investigator captured another video of the participant's movements. The investigator started a 24-hour long EEG of the participant. The investigator conducted an unstructured interview with the caregiver of the participant. The participant was evaluated by a developmental pediatrician, neuropsychologist, or psychologist, in the same fashion as during visit 5.

Visit 15 was the day after visit 14. During visit 15, the investigator reviewed the EEG video, noting the number of seizures, bruxism patterns, and clinical interpretation. The investigator reviewed the participant's seizure diary, medical and surgical history, concomitant medications, and clinical assessment. The investigator administered the side effects questionnaire and captured another video of the participant's movements. The investigator conducted an unstructured interview with the caregiver of the participant.

Visits 16-20 were used to monitor the participant after the medication was tapered. The daily dose of medication was reduced by 10% each day over 10 days until medication administration was ended at visit 16. Visits 16-20 occurred approximately 0, 1, 2, 3, and 4 weeks after visits 14-15. These visits were conducted by phone or video. The investigator reviewed the participant's seizure diary, medical and surgical history, concomitant medications, and clinical assessment. The investigator administered the side effects questionnaire, the adherence questionnaire/medication diary, and the PELHS QOL-2. The investigator conducted an unstructured interview with the caregiver of the participant.

Some families opted to continue taking phenylbutyrate and were subject to extended follow up visits. For example, some participants continued taking the medication if symptoms worsened on discontinuation of the medication.

If a participant underwent sedation for any reason during the study period (including the “extended follow up” period), the investigators asked for additional consent to perform a lumbar puncture for a cerebrospinal fluid (CSF) sample. (For example, a child might have undergone sedation if the child needed a gastrostomy tube placed, or the child needed a sedated MRI, or the child had a tendon-release surgery for spasticity.) As part of this research protocol, the participants were not sedated for the sole purpose of a lumbar puncture. The rationale was that direct measurement of CSF concentration of phenylbutyrate was helpful for future study design—i.e., to understand if the investigators were able to achieve CSF concentrations of 0.5-1 mM as described above.

### Outcomes

### Measurement of Effect

Response to the medication was measured qualitatively and quantitatively using the following measures. Quantitative and qualitative measures included the change in the number of and percent change in the seizures per week, as recorded in the seizure diary, comparing weeks 1-4 with weeks 5-10; change and percent change in number of seizures per hour, as recorded by video EEG, comparing visit 5 (day before medication) to visit 6 (first day of medication), and comparing visit 5 to visit 14 (40 days after the first day of medication); and changes in bruxism patterns in EEG data, comparing visit 5 to visit 6 and visit 5 to visit 14. Additional measures comparing visit 5 and visit 14 included quality of life assessment, as quantified by the change in PEDSQL epilepsy module score, and by the trajectory and overall change in the PELHS QOL-2 score; qualitative analysis of the participant's development and behavior; change in developmental assessment scores; ataxia as measured by changes in SARA scale score; and sleep as measured by CSHQ score. Medication dosing adherence was also assessed, quantified as the percentage of days in which the participant received the full prescribed dose, and considering obstacles to adherence qualitatively as reported by caregivers. There were also post hoc assessments of changes in EEG, changes in movement disorder, and parents' experiences with the medication. The percentage of patients who improved in seizure frequency by 50% or more were also noted.

### Results

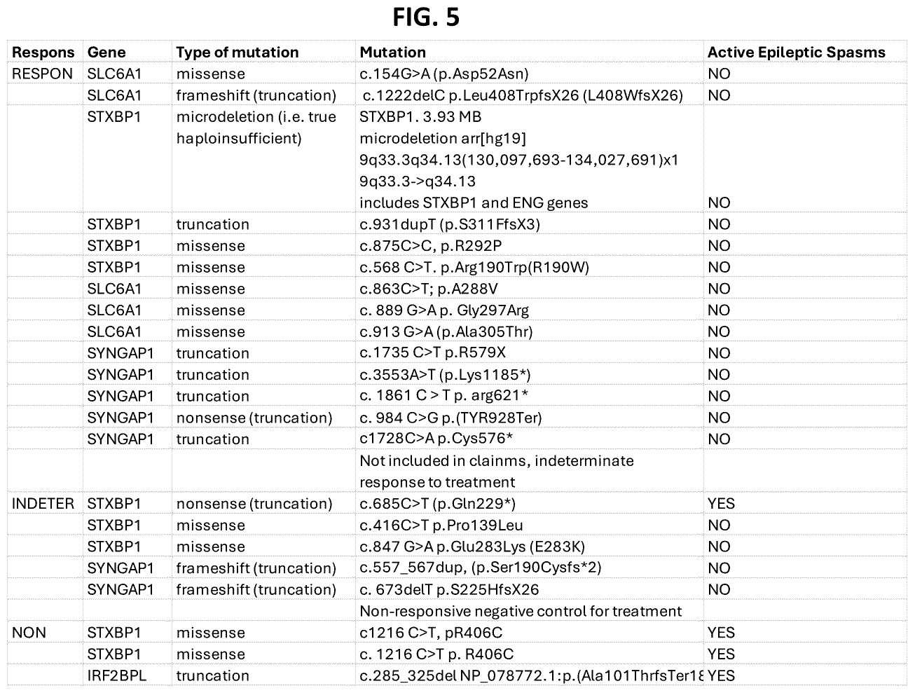

FIG. 5 is a table of the results of the study described in the Examples section below. Responders were participants that had 50% or more reduction in seizures that was clinically associated with the use of glycerol phenylbutyrate. Indeterminant response meant there was improvement, but it was either less than 50% reduction in seizures or not clinically associated with the use of glycerol phenylbutyrate. Of 20 participants, 11 participants were responders, 3 participants had an indeterminant response, 2 participants were non-responders, 4 participants had no seizures.

In terms of nonseizure outcomes, as assessed at 10 weeks of drug exposure, 8 of 11 participants had improved communication, as assessed with the ORCA. 5 of 12 participants had improved sleep, as assessed with the CSHQ. 4 of 11 participants had improved quality of life, as assessed with the PEDSQL.

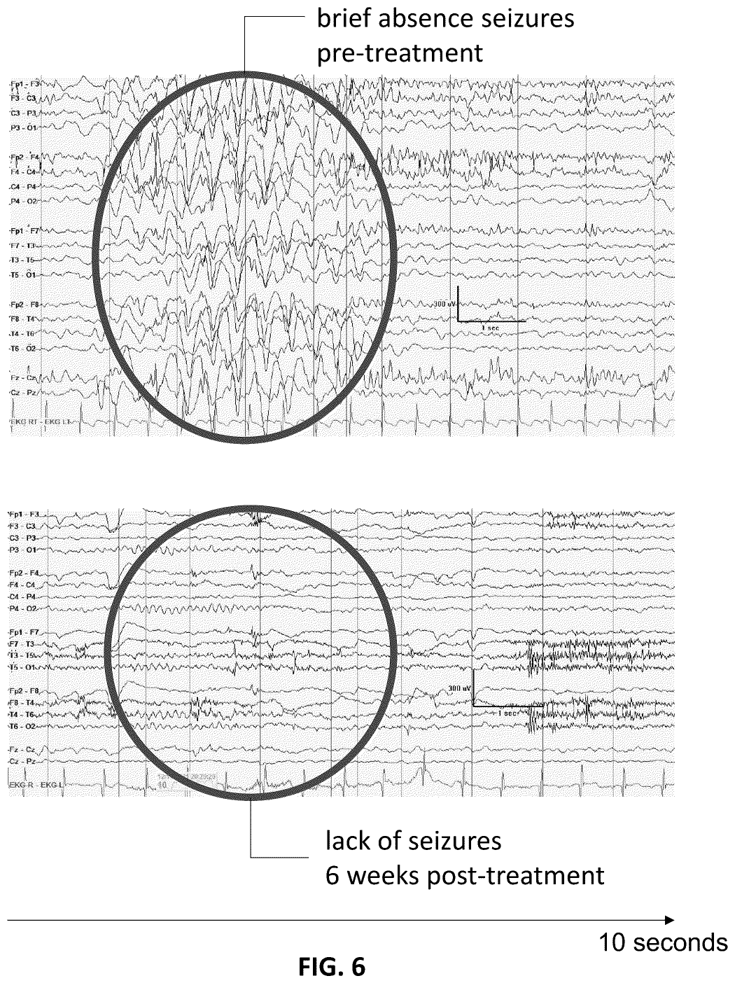

FIG. 6 show EEGs prior to treatment (top) and six weeks after treatment with glycerol phenylbutyrate (bottom). Each EEG was 10 seconds long and was collected from the same child with SLC6A1. There were substantial changes in EEGs after treatment with glycerol phenylbutyrate. The circled region in the top EEG represents a seizure, called a brief absence seizure. The EEG after treatment (bottom), did not show seizure activity.

These results demonstrate that the methods of the present technology are useful for selecting a patient diagnosed with or at risk for STXBP1 encephalopathy (STXBP1-E) for treatment with a composition including phenylbutyrate or a pharmaceutically acceptable salt thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all FIGS. and tables, to the extent they are not inconsistent with the explicit teachings of this specification.

1. A method for treating STXBP1 encephalopathy (STXBP1-E) in a patient in need thereof comprising administering an effective amount of a composition including phenylbutyrate or a pharmaceutically acceptable salt thereof to the patient, wherein the patient comprises a STXBP1 haploinsufficient allele, optionally wherein the composition comprises glycerol phenylbutyrate.

2. The method of claim 1, wherein the STXBP1 haploinsufficient allele comprises a nonsense mutation, a missense mutation, a deletion, an inversion or a frameshift mutation or wherein the STXBP1 haploinsufficient allele is S311FfsX3, R292P, or R190W.

3. A method for treating SYNGAP1 encephalopathy in a patient in need thereof comprising administering an effective amount of a composition including phenylbutyrate or a pharmaceutically acceptable salt thereof to the patient, wherein the patient comprises a SYNGAP1 haploinsufficient allele.

4. The method of claim 3, wherein the SYNGAP1 haploinsufficient allele comprises a nonsense mutation, a missense mutation, a deletion, an inversion or a frameshift mutation or wherein the SYNGAP1 haploinsufficient allele is R579X, K1185*, R621*, Y928*, or C576*.

5. The method of claim 1, wherein the patient does not comprise a dominant-negative STXBP1 mutation, optionally wherein the dominant-negative STXBP1 mutation is selected from the group consisting of V84D, C180Y, L183R, P335L, R406C, R406H, M443R, P480L, G544D, G544V, R551C, C552R, and T574R.

6. The method of claim 1, wherein the patient does not comprise: one or more STXBP1 mutations selected from among P139L, E283K, and Q229*, one or more SYNGAP 1 mutations selected from among S190Cysfs*2 or S225HfsX26, or a IRF2BPL mutation comprising A101ThrfsTer18.

7. The method of claim 1, wherein the STXBP1-E presents as infantile spasms, epilepsy of infancy migrating focal seizure, Dravet syndrome or non-syndromic epilepsy.

8. The method of claim 1, wherein the patient does not exhibit active epileptic spasms.

9. The method of claim 1, wherein the patient exhibits one or more of autism, low tone, movement disorders (including ataxia or bruxism), abnormal EEGs (>60% with focal or multifocal epileptiform discharges), or abnormal MRI brain imaging (including atrophy, thin corpus callosum, or delayed myelination).

10. A method for treating developmental and epileptic encephalopathy (DEE) in a patient in need thereof comprising administering an effective amount of a composition including phenylbutyrate or a pharmaceutically acceptable salt thereof to the patient, wherein the patient does not exhibit active epileptic spasms, optionally wherein the composition comprises glycerol phenylbutyrate.

11. The method of claim 10, wherein the patient comprises a STXBP1 haploinsufficient allele, a SYNGAP1 haploinsufficient allele, and/or a SLC6A1 haploinsufficient allele.

12. The method of claim 11, wherein the STXBP1 haploinsufficient allele, SYNGAP1 haploinsufficient allele, or SLC6A1 haploinsufficient allele comprises a nonsense mutation, a missense mutation, a deletion, an inversion or a frameshift mutation.

13. The method of claim 11, wherein the STXBP1 haploinsufficient allele is S311FfsX3, R292P, or R190W.

14. The method of claim 11, wherein the SLC6A1 haploinsufficient allele is selected from among D52N, L408WfsX26, A288V, V125M, S295L, G362R, D410E, L460R and W495X.

15. The method of claim 11, wherein the SYNGAP1 haploinsufficient allele is R579X, K1185*, R621*, Y928Ter, or C576*.

16. The method of claim 10, wherein the patient does not comprise one or more STXBP1 mutations selected from the group consisting of V84D, C180Y, L183R, P335L, R406C, R406H, M443R, P480L, G544D, G544V, R551C, C552R, T574R, P139L, E283K, and Q229*, one or more SYNGAP 1 mutations selected from among S190Cysfs*2 or S225HfsX26, or a IRF2BPL mutation comprising A101ThrfsTer18.

17. The method of claim 1, wherein the patient is an infant, a child or an adolescent.

18. The method of claim 1, wherein the composition including phenylbutyrate or the pharmaceutically acceptable salt thereof is administered enterally, optionally wherein the composition is administered via a nasogastric or gastrostomy tube.

19. The method of claim 1, wherein the composition is administered daily for at least 6 weeks, optionally wherein the composition is administered thrice a day.

20. The method of claim 1, wherein administration of the composition results in amelioration of one or more of reduction in seizures, improvement in cognition, reduction in abnormal movement, improvement in sleep quality, improved gait, improved quality life, improved mood, reduction in bruxism, improvement in gross motor skills, improvement in fine motor skills, improvement in communication skills, or reduction in family burden.


SEQUENCE LISTING (19 sequences)

SEQ ID NO: 1 - DNA, 3754 bp, Homo sapiens
  source 1..3754: mol_type = genomic DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 2 - DNA, 3745 bp, Homo sapiens
  source 1..3745: mol_type = genomic DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 3 - DNA, 3975 bp, Homo sapiens
  source 1..3975: mol_type = genomic DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagagaaagaggaagactacctagacatgttgctccagagcagaatttccagcagtagattgaaccttcggaggccgagtcccagcccagtgtaaactgagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggttctactcacattcttactcccaccaaatttctcatggacctgagacaccccgacttcagggagtcctctagggtatcttttgaggatcaggctccaacaatggagtgagagccaaagaaacaaagatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 4 - DNA, 4070 bp, Homo sapiens
  source 1..4070: mol_type = genomic DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagagatatatacctaggaatggagttgctggatctctatgtttggttatttgaggagctgccggcctgttttccacatcagctgtaccattttacattctcaccagcagtgcatgacggtccagtgcctttttatcttactgcgcttaaggtgtaattccttctttctcagactggactgtaaattctttgagagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggttctactcacattcttactcccaccaaatttctcatggacctgagacaccccgacttcagggagtcctctagggtatcttttgaggatcaggctccaacaatggagtgagagccaaagaaacaaagatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 5 - DNA, 3726 bp, Homo sapiens
  source 1..3726: mol_type = genomic DNA
agaaagaggaaagacaggacccgagcggggtttcaggcagatggagcgcgtcggtagcctgtggccagggatcccagcaccgacgggaaagaggaggcctgggtaccctgcgccccgggcgcgcgcggcgcgtgagagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 6 - DNA, 3944 bp, Homo sapiens
  source 1..3944: mol_type = genomic DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagagatatatacctaggaatggagttgctggatctctatgtttggttatttgaggagctgccggcctgttttccacatcagctgtaccattttacattctcaccagcagtgcatgacggtccagtgcctttttatcttactgcgcttaaggtgtaattccttctttctcagactggactgtaaattctttgagagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 7 - DNA, 3849 bp, Homo sapiens
  source 1..3849: mol_type = genomic DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagagaaagaggaagactacctagacatgttgctccagagcagaatttccagcagtagattgaaccttcggaggccgagtcccagcccagtgtaaactgagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 8 - DNA, 3856 bp, Homo sapiens
  source 1..3856: mol_type = genomic DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagagaaagaggaagactacctagacatgttgctccagagcagaatttccagcagtagattgaaccttcggaggccgagtcccagcccagtgtaaactggtaaagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 9 - DNA, 3834 bp, Homo sapiens
  source 1..3834: mol_type = genomic DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggggaatggcccttcagaaccttggctaggtgacatcagaagctgcttgtgtatgtaaggaaaatggggcttcctcctaaggatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 10 - DNA, 2865 bp, Homo sapiens
  source 1..2865: mol_type = genomic DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgatagtgcccgtggaggcgggaagctgagcccggctcccaggaagcacagtgagggctcagccattgatgatgatgatgatgatgatgatgatgatgatgatgatggtgatgatgatgatgctgctgctgttgacgatgagtccaagccaagcctggggagaaaagaataaatagtctcataaatttaaaaaagcccaatgctcatcaagaagagaacagataaactgtgtattcacccaatggtgagtttaaagcagttaaaaatgcatgacacacagttgcatatgtcaacatgaatagatctcagacacttaacaatgagtgaaaagagcacattatgggaggacacagacaggatagtgcatttatataaagcttcaatgttgcaggctgaaagagtgagggtcgtgatcaactcagtatcctggaggctacatgggtaaacagcaaactgttctcataaatgcagaatgttggcaaactgacaaactgcatctgccgcccagaaggaatgcggagggcagccactccctaagcgcagttttcttgtgattaggtacatctgaagcctgttagcaataatgtgaacctgtgatcaattaagcagctgaccaatcattacctcctcttccctgctctttctacccagtaaatacaaagggctgtagaagctcagagctgctgcttttgctcagtagaagcagggagtcctcttcttcttcccccgaccccttcttttaaaacagtttcttttaagttttcatttctgcgttcatcctccttcattcagtcccgtagtaaccgtggcaaaccacggcacttcaaaacctcataaaaagcaccacatattgcttatttggatacatatgttgtaaatgtgtgaaaacatgcatgggaatgacaaataccaaagtcaggatagtgactggccatggggaagggatggttattattgtcgtcttatatcttcaattccaaatgagccacgggcttaaaagtacttcattgaataaaaaaaaaccaacaatgtattaatgcaa

SEQ ID NO: 11 - DNA, 3772 bp, Homo sapiens
  source 1..3772: mol_type = genomic DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggttctactcacattcttactcccaccaaatttctcatggacctgagacaccccgacttcagggagtcctctagggtatcttttgaggatcaggctccaacaatggagtgagagccaaagaaacaaagatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 12 - DNA, 3880 bp, Homo sapiens
  source 1..3880: mol_type = genomic DNA
agtcggtccctagcgcggctgcggggcggagagctgcggctggcccagcgcgcccacctgaggaggcggcggggtccgcaggcgtcgcgggacgaggagatcggagccgggagactcgcgcagcgccatggcccccattggcctcaaagctgttgtcggagagaagattatgcatgatgtgataaagaaggtcaagaagaagggggaatggaaggtgctggtggtggatcagttaagcatgaggatgctgtcctcctgctgcaagatgacagacatcatgaccgagggcataacgattgtggaagatatcaataagcgcagagagccgctccccagcctggaggctgtgtatctcatcactccatccgagaagtccgtccactctctcatcagtgactttaaggacccgccgactgctaaataccgggctgcacacgtcttcttcactgactcttgtccagatgccctgtttaatgaactggtaaaatcccgagcagccaaagtcatcaaaactctgacggaaatcaatattgcatttctcccgtatgaatcccaggtctattccttggactctgctgactctttccaaagcttctacagtccccacaaggctcagatgaagaatcctatactggagcgcctggcagagcagatcgcgaccctttgtgccaccctgaaggagtacccggctgtgcggtatcggggggaatacaaggacaatgccctgctggctcagctaatccaggacaagctcgatgcctataaagctgatgatccaacaatgggggagggcccagacaaggcacgctcccagctcctgatcctggatcgaggctttgaccccagctcccctgtgctccatgaattgacttttcaggctatgagttatgatctgctgcctatcgaaaatgatgtatacaagtatgagaccagcggcatcggggaggcacgggtgaaggaggtgctcctggacgaggacgacgacctgtggatagcactgcgccacaagcacatcgcagaggtgtcccaggaagtcacccggtctctgaaagatttttcttctagcaagagaatgaatactggagagaagaccaccatgcgggacctgtcccagatgctgaagaagatgcctcagtaccagaaagagctcagcaagtactccacccacctgcaccttgctgaggactgtatgaagcattaccaaggcaccgtagacaaactctgccgagtggagcaggacctggccatgggcacagatgctgagggagagaagatcaaggaccctatgcgagccatcgtccccattctgctggatgccaatgtcagcacttatgacaaaatccgcatcatccttctctacatctttttgaagaatggcatcacggaggaaaacctgaacaaactgatccagcacgcccagatacccccggaggatagtgagatcatcaccaacatggctcacctcggcgtgcccatcgtcaccgattccacgctgcgtcgccggagcaagccggagcggaaggaacgcatcagcgagcagacctaccagctctcacggtggactccgattatcaaggacatcatggaggacactattgaggacaaacttgacaccaaacactacccttatatctctacccgttcctctgcctccttcagcaccaccgccgtcagcgcccgctatgggcactggcataagaacaaggccccaggcgagtaccgcagtggcccccgcctcatcattttcatccttgggggtgtgagcctgaatgagatgcgctgcgcctacgaggtgacccaggccaacggaaagtgggaggtgctgataggttctactcacattcttactcccaccaaatttctcatggacctgagacaccccgacttcagggagtcctctagggtatcttttgaggatcaggctccaacaatggagtgagagccaaagaaacaaagatccacacacatcctcaccccacagaaactgctggacacactgaagaaactgaataaaacagatgaagaaataagcagttaaaaaaataagtcgcccctccaaaacacgcccccatcccacagcgctccgcagcttcccaccaccgcccgcctcagttcctttgcgtctgttgcctccccagccctgcacgccctggctggcactgttgccgctgcattctcgtgttcagtgatgccctcttcttgtttgaaacaaaagaaaataatgcattgtgttttttaaaaagagtatcttatacatgtatcctaaaaagagaagctcatgtgcaattggtgcacagcaggagaaatttctggactgttaggatgaatggacgccttctccccgttatttaagatttgtgaccttgtacataaccctgggtgacgtgcacattgcttgggtatggaacggtagaaatttgggtgtttttaaaaccttgtttggggttgttcctgtccttgttgagaatcatagagatgtctgtgttcttggagtatttcacactgaggactaatctgctatcttcattccagtccctacccctcagtgcctgctctcatccaaataacctgggaggtgacaatcaggatatctcaggaggtccaaggtggaacagacctctttgcctttcccagcgtctcatacccccggtagtgcagctgtgggtggaggctggggtgtctgcacgaagtcaggccagcgtcctcctccacagcctgtcactgccccctccccagcctgtgtccacagtgctgtgatcccgagggaagtcctccagtctaagtcacagtgccctgacaggtgagaagcaaactcccgctggaagcctccatctctttggaaaaacagttagtctggagcctgtggcccaggcccttctgtccccaggcatcatcccaacagctcattttccctagtccgccttcgttcaagggtcaggaatggaccagaacagatgggttctggaggcccctgaacagagggctatggctgtggagaaggttcttggcccgttggactcacacagaccctgtaccctctcggcaagcatcttcagtcagattatcctcagtttcagatacttcataataccttgtgttgtgtggggtcatacatcatcgtgtttgtaagagaagatggtcattttattctctgtataaaacttagctctaaagcagaaactaaagcagcaaatgcaggaaggctgtctcgccatcctcaagactcagcagctctcattctccagtggtgagcacaccatttgtgctgctgctgttgtcgtgaaatataataacagtggaagtcacaaaaatgtcccctgcccagccccctcgccgcccttgacctcctgcaggccatgtgtgtattacttgtctagtgatgtcctctcaaagtgctgtacgcgagctcggcgccacctccgcctccctttcagagcctgctccccgccctctctgctcgctgcattgtggtgttctcttctcaaggctttgaaatctccccttgcactgagattagtcgtcagatctctccccgtctccctcccaacttatacgacctgatttccttaggacggaaccgcaggcacctgcgccgggcgtcttactcccgctgcttgttctgtcccctccctcggaccaaacagtgctcatgcttcaggaccttgtttgtcgaagatgttggtttccctttctctgttatttatataaaaataatttatcaaaaggatattttaaaaaagctagtctgtcttgaaacttgtttaccttaaaattatcagaatctcagtgtttgaaagtactgaagcacaaacatatatcatctctgtaccattctgtactaaagcacttgagtctaataaataaagaaatcagcacccct

SEQ ID NO: 13 - DNA, 4417 bp, Homo sapiens
  source 1..4417: mol_type = genomic DNA
gccctcggaagaccgagacagcggagaggttgcgggtgagctgcgctgagcccaggagccgaggagtcgggagcgcagtagcgctgagcccgagcccgagcggccccgcgtcccgagcgcatcggagcggccgagccgcccggatgcagcgcctgtcccgggcagcgcagccccggccgcaggatctcacccagggtggcagaaggaggccttctggagctgacccacccccgacgaccatcagggtgcccttgagccgcaaaactgctgtccacgtggaccgggggtgacatcgcacgtccatctgccaggacccctgcgtccaaattccgagacatggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagggtgggggccactcaccgacccgacactctcaccccccgacctggctgagtgcgaccaccacttgatgtctgaggataccttccatctcaacctacctcgagtggcgagtccagacaccatcaccacgcagagaggggaggtgggaggacagttagacccctgggtgggccctgccgtgggcaaggatacccggtggcttctggcacctggcgggctggtgacctttttaatccaggccccatcagcatcccacgatcggccttggtaaccgccgcggtagatcatttttatcccgccagggagtgtgatgcaggaagaccacatgcgctcctggcttttaaacctgttcctgactgttctcttactgccgaaacccttgactgttatctcggactttgcaggagttcctttccctccgaacgctgctccatgcacaggaaaagggcattttgtacaatggggacttcccgggaacgcttgctcttaagtaccagaagccggcggagctctggctttcgtgtttttggttttctccttcccaaggcagctggattgaaaaaacaaaacaaaacaaaaaaacccaggggcgtcagtcgatattcccagggccgcttctcctgcagtctgtggagcgtccttgtccccgccgccggaatgaatgagcattctgcagcccgatgtccctgtcccctcctcgccgggccattctgattggacctggcccagtgcaatctgtccagacaagccctgcttgctggaaaactgccacaagcacaattgatctctttttatcgccattccaggggcctcaggtcctactggggaaacttcctataccggagctccagtttctcttaagctgcccaatttcacagagtacaaaatagttgtaggggaaatcaaggtgaaggatctgtccgacagtcaagacggatccacagtaatctttcggtctccttaaactaccaccctcgctgccacccaccccaagctgctgccgcctcaccttccttgaaatttctcagcgggagtctcctcactgccactaaaatccacccagcccactaactgaggagctagtgttaatccagagaaccccccgcaatgtgcttccgagattcagactgcttcattgggaagtatgatttgttcctttctggaattgggctccgtggtggcggcggcacttcaagcaaagacagtttcttgcaagctccagtagctccgcgtgtctcatttgccaggaagatgggttcccacgtagcaaatcgtacattgtgccctgtagctccttagctagttagctcacaagccgtgttttatgactaatccttaataactatggtaaataactgtgactgtggggtttttaatctcttgtcattctcatccaaaagtgaccagcataccagttcttgcaataagatattaccctcagaatattaagcacattattgtagagaaaaaaaaatatgtgtacacatatgaacgcacaacatgcacattcatcctcacatgtggcacgtaaggtctcatttgatattgtgtaggaaatctgaagccttttcctgaggtcatctgtaaaatagtctcattgccaaggcatccccagtgccagctggtgaatccatgatcaaaatgcatacgtattgttaaatgataaggtttagaatgacaggaacccatcactgtgtctcatggtcccacttccccatctgtgtgtgaattcctttagactaagggcaggaagacttccagctttctctttgttcttcaatgtgaaactgagaccaagtctctctaagacaaatgcagtgtatttaatgtttgtaagcaattctaagtgagatgtttggcaagaaatcccctaactgatttccatccaaacctaccttatagagcacaatattaagtgttgtacaattactgtgagaactgtgaatatgtgtaacttttttttagtatttgcccggggggaaaaagatattgtattatcatatatgcttttttgcaataaggatttattctcagaacaccaagtaaatctatctctatataaaaaatatatgtaatatatacatattcaaagtatatacagagcctgttttaaaaaatacagtattatttagtaaaattatctgttctatggaccaaatgtaaaatatttataaatgaagatgcattttaaatgtctataaatggtgtcataactagagcacgggcgttatgtaagtttctaagaatttagaggataaataataaaggttctatgatatacaa

SEQ ID NO: 14 - DNA, 4227 bp, Homo sapiens
  source 1..4227: mol_type = genomic DNA
gccctcggaagaccgagacagcggagaggttgcgggtgagctgcgctgagcccaggagccgaggagtcgggagcgcagtagcgctgagcccgagcccgagcggccccgcgtcccgagcgcatcggagcggccgagccgcccggatgcagcgcctgtcccgggcagcgcagccccggccgcaggatctcacccagggtggcagaaggaggccttctggagctgacccacccccgacgaccatcagggtgcccttgagccgcaaaactgctgtccacgtggaccgggggtgacatcgcacgtccatctgccaggacccctgcgtccaaattccgagacatggcgaccaacggcagcaaggtggccgacgggcagatctccaccgaggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagggtgggggccactcaccgacccgacactctcaccccccgacctggctgagtgcgaccaccacttgatgtctgaggataccttccatctcaacctacctcgagtggcgagtccagacaccatcaccacgcagagaggggaggtgggaggacagttagacccctgggtgggccctgccgtgggcaaggatacccggtggcttctggcacctggcgggctggtgacctttttaatccaggccccatcagcatcccacgatcggccttggtaaccgccgcggtagatcatttttatcccgccagggagtgtgatgcaggaagaccacatgcgctcctggcttttaaacctgttcctgactgttctcttactgccgaaacccttgactgttatctcggactttgcaggagttcctttccctccgaacgctgctccatgcacaggaaaagggcattttgtacaatggggacttcccgggaacgcttgctcttaagtaccagaagccggcggagctctggctttcgtgtttttggttttctccttcccaaggcagctggattgaaaaaacaaaacaaaacaaaaaaacccaggggcgtcagtcgatattcccagggccgcttctcctgcagtctgtggagcgtccttgtccccgccgccggaatgaatgagcattctgcagcccgatgtccctgtcccctcctcgccgggccattctgattggacctggcccagtgcaatctgtccagacaagccctgcttgctggaaaactgccacaagcacaattgatctctttttatcgccattccaggggcctcaggtcctactggggaaacttcctataccggagctccagtttctcttaagctgcccaatttcacagagtacaaaatagttgtaggggaaatcaaggtgaaggatctgtccgacagtcaagacggatccacagtaatctttcggtctccttaaactaccaccctcgctgccacccaccccaagctgctgccgcctcaccttccttgaaatttctcagcgggagtctcctcactgccactaaaatccacccagcccactaactgaggagctagtgttaatccagagaaccccccgcaatgtgcttccgagattcagactgcttcattgggaagtatgatttgttcctttctggaattgggctccgtggtggcggcggcacttcaagcaaagacagtttcttgcaagctccagtagctccgcgtgtctcatttgccaggaagatgggttcccacgtagcaaatcgtacattgtgccctgtagctccttagctagttagctcacaagccgtgttttatgactaatccttaataactatggtaaataactgtgactgtggggtttttaatctcttgtcattctcatccaaaagtgaccagcataccagttcttgcaataagatattaccctcagaatattaagcacattattgtagagaaaaaaaaatatgtgtacacatatgaacgcacaacatgcacattcatcctcacatgtggcacgtaaggtctcatttgatattgtgtaggaaatctgaagccttttcctgaggtcatctgtaaaatagtctcattgccaaggcatccccagtgccagctggtgaatccatgatcaaaatgcatacgtattgttaaatgataaggtttagaatgacaggaacccatcactgtgtctcatggtcccacttccccatctgtgtgtgaattcctttagactaagggcaggaagacttccagctttctctttgttcttcaatgtgaaactgagaccaagtctctctaagacaaatgcagtgtatttaatgtttgtaagcaattctaagtgagatgtttggcaagaaatcccctaactgatttccatccaaacctaccttatagagcacaatattaagtgttgtacaattactgtgagaactgtgaatatgtgtaacttttttttagtatttgcccggggggaaaaagatattgtattatcatatatgcttttttgcaataaggatttattctcagaacaccaagtaaatctatctctatataaaaaatatatgtaatatatacatattcaaagtatatacagagcctgttttaaaaaatacagtattatttagtaaaattatctgttctatggaccaaatgtaaaatatttataaatgaagatgcattttaaatgtctataaatggtgtcataactagagcacgggcgttatgtaagtttctaagaatttagaggataaataataaaggttctatgatatacaa

SEQ ID NO: 15 - DNA, 4156 bp, Homo sapiens
  source 1..4156: mol_type = genomic DNA
gccctcggaagaccgagacagcggagaggttgcgggtgagctgcgctgagcccaggagccgaggagtcgggagcgcagtagcgctgagcccgagcccgagcggccccgcgtcccgagcgcatcggagcggccgagccgcccggatgcagcgcctgtcccgggcagcgcagccccggccgcaggatctcacccagggtggcagaaggaggccttctggagctgacccacccccgacgaccatcagggtgaggcaactccaaggtcctactctctttctgtgcctgttacccaccccgtcctcctagggtgcccttgagccgcaaaactgctgtccacgtggaccgggggtgacatcgcacgtccatctgccaggacccctgcgtccaaattccgagacatggcgaccaacggcagcaaggtggccgacgggcagatctccaccgaggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagggtgggggccactcaccgacccgacactctcaccccccgacctggctgagtgcgaccaccacttgatgtctgaggataccttccatctcaacctacctcgagtggcgagtccagacaccatcaccacgcagagaggggaggtgggaggacagttagacccctgggtgggccctgccgtgggcaaggatacccggtggcttctggcacctggcgggctggtgacctttttaatccaggccccatcagcatcccacgatcggccttggtaaccgccgcggtagatcatttttatcccgccagggagtgtgatgcaggaagaccacatgcgctcctggcttttaaacctgttcctgactgttctcttactgccgaaacccttgactgttatctcggactttgcaggagttcctttccctccgaacgctgctccatgcacaggaaaagggcattttgtacaatggggacttcccgggaacgcttgctcttaagtaccagaagccggcggagctctggctttcgtgtttttggttttctccttcccaaggcagctggattgaaaaaacaaaacaaaacaaaaaaacccaggggcgtcagtcgatattcccagggccgcttctcctgcagtctgtggagcgtccttgtccccgccgccggaatgaatgagcattctgcagcccgatgtccctgtcccctcctcgccgggccattctgattggacctggcccagtgcaatctgtccagacaagccctgcttgctggaaaactgccacaagcacaattgatctctttttatcgccattccaggggcctcaggtcctactggggaaacttcctataccggagctccagtttctcttaagctgcccaatttcacagagtacaaaatagttgtaggggaaatcaaggtgaaggatctgtccgacagtcaagacggatccacagtaatctttcggtctccttaaactaccaccctcgctgccacccaccccaagctgctgccgcctcaccttccttgaaatttctcagcgggagtctcctcactgccactaaaatccacccagcccactaactgaggagctagtgttaatccagagaaccccccgcaatgtgcttccgagattcagactgcttcattgggaagtatgatttgttcctttctggaattgggctccgtggtggcggcggcacttcaagcaaagacagtttcttgcaagctccagtagctccgcgtgtctcatttgccaggaagatgggttcccacgtagcaaatcgtacattgtgccctgtagctccttagctagttagctcacaagccgtgttttatgactaatccttaataactatggtaaataactgtgactgtggggtttttaatctcttgtcattctcatccaaaagtgaccagcataccagttcttgcaataagatattaccctcagaatattaagcacattattgtagagaaaaaaaaatatgtgtacacatatgaacgcacaacatgcacattcatcctcacatgtggcacgtaaggtctcatttgatattgtgtaggaaatctgaagccttttcctgaggtcatctgtaaaatagtctcattgccaaggcatccccagtgccagctggtgaatccatgatcaaaatgcatacgtattgttaaatgataaggtttagaatgacaggaacccatcactgtgtctcatggtcccacttccccatctgtgtgtgaattcctttagactaagggcaggaagacttccagctttctctttgttcttcaatgtgaaactgagaccaagtctctctaagacaaatgcagtgtatttaatgtttgtaagcaattctaagtgagatgtttggcaagaaatcccctaactgatttccatccaaacctaccttatagagcacaatattaagtgttgtacaattactgtgagaactgtgaatatgtgtaacttttttttagtatttgcccggggggaaaaagatattgtattatcatatatgcttttttgcaataaggatttattctcagaacaccaagtaaatctatctctatataaaaaatatatgtaatatatacatattcaaagtatatacagagcctgttttaaaaaatacagtattatttagtaaaattatctgttctatggaccaaatgtaaaatatttataaatgaagatgcattttaaatgtctataaatggtgtcataactagagcacgggcgttatgtaagtttctaagaatttagaggataaataataaaggttctatgatatacaa

SEQ ID NO: 16 - DNA, 4095 bp, Homo sapiens
  source 1..4095: mol_type = genomic DNA
gccctcggaagaccgagacagcggagaggttgcgggtgagctgcgctgagcccaggagccgaggagtcgggagcgcagtagcgctgagcccgagcccgagcggccccgcgtcccgagcgcatcggagcggccgagccgcccggatgcagcgcctgtcccgggcagcgcagccccggccgcaggatctcacccagggtggcagaaggaggccttctggagctgacccacccccgacgaccatcagggtgcccttgagccgcaaaactgctgtccacgtggaccgggggtgacatcgcacgtccatctgccaggacccctgcgtccaaattccgagacatggcgaccaacggcagcaaggtggccgacgggcagatctccaccgaggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagggtgggggccactcaccgacccgacactctcaccccccgacctggctgagtgcgaccaccacttgatgtctgaggataccttccatctcaacctacctcgagtggcgagtccagacaccatcaccacgcagagaggggaggtgggaggacagttagacccctgggtgggccctgccgtgggcaaggatacccggtggcttctggcacctggcgggctggtgacctttttaatccaggccccatcagcatcccacgatcggccttggtaaccgccgcggtagatcatttttatcccgccagggagtgtgatgcaggaagaccacatgcgctcctggcttttaaacctgttcctgactgttctcttactgccgaaacccttgactgttatctcggactttgcaggagttcctttccctccgaacgctgctccatgcacaggaaaagggcattttgtacaatggggacttcccgggaacgcttgctcttaagtaccagaagccggcggagctctggctttcgtgtttttggttttctccttcccaaggcagctggattgaaaaaacaaaacaaaacaaaaaaacccaggggcgtcagtcgatattcccagggccgcttctcctgcagtctgtggagcgtccttgtccccgccgccggaatgaatgagcattctgcagcccgatgtccctgtcccctcctcgccgggccattctgattggacctggcccagtgcaatctgtccagacaagccctgcttgctggaaaactgccacaagcacaattgatctctttttatcgccattccaggggcctcaggtcctactggggaaacttcctataccggagctccagtttctcttaagctgcccaatttcacagagtacaaaatagttgtaggggaaatcaaggtgaaggatctgtccgacagtcaagacggatccacagtaatctttcggtctccttaaactaccaccctcgctgccacccaccccaagctgctgccgcctcaccttccttgaaatttctcagcgggagtctcctcactgccactaaaatccacccagcccactaactgaggagctagtgttaatccagagaaccccccgcaatgtgcttccgagattcagactgcttcattgggaagtatgatttgttcctttctggaattgggctccgtggtggcggcggcacttcaagcaaagacagtttcttgcaagctccagtagctccgcgtgtctcatttgccaggaagatgggttcccacgtagcaaatcgtacattgtgccctgtagctccttagctagttagctcacaagccgtgttttatgactaatccttaataactatggtaaataactgtgactgtggggtttttaatctcttgtcattctcatccaaaagtgaccagcataccagttcttgcaataagatattaccctcagaatattaagcacattattgtagagaaaaaaaaatatgtgtacacatatgaacgcacaacatgcacattcatcctcacatgtggcacgtaaggtctcatttgatattgtgtaggaaatctgaagccttttcctgaggtcatctgtaaaatagtctcattgccaaggcatccccagtgccagctggtgaatccatgatcaaaatgcatacgtattgttaaatgataaggtttagaatgacaggaacccatcactgtgtctcatggtcccacttccccatctgtgtgtgaattcctttagactaagggcaggaagacttccagctttctctttgttcttcaatgtgaaactgagaccaagtctctctaagacaaatgcagtgtatttaatgtttgtaagcaattctaagtgagatgtttggcaagaaatcccctaactgatttccatccaaacctaccttatagagcacaatattaagtgttgtacaattactgtgagaactgtgaatatgtgtaacttttttttagtatttgcccggggggaaaaagatattgtattatcatatatgcttttttgcaataaggatttattctcagaacaccaagtaaatctatctctatataaaaaatatatgtaatatatacatattcaaagtatatacagagcctgttttaaaaaatacagtattatttagtaaaattatctgttctatggaccaaatgtaaaatatttataaatgaagatgcattttaaatgtctataaatggtgtcataactagagcacgggcgttatgtaagtttctaagaatttagaggataaataataaaggttctatgatatacaa

SEQ ID NO: 17 - DNA, 4478 bp, Homo sapiens
  source 1..4478: mol_type = genomic DNA
gccctcggaagaccgagacagcggagaggttgcgggtgagctgcgctgagcccaggagccgaggagtcgggagcgcagtagcgctgagcccgagcccgagcggccccgcgtcccgagcgcatcggagcggccgagccgcccggatgcagcgcctgtcccgggcagcgcagccccggccgcaggatctcacccagggtggcagaaggaggccttctggagctgacccacccccgacgaccatcagggtgaggcaactccaaggtcctactctctttctgtgcctgttacccaccccgtcctcctagggtgcccttgagccgcaaaactgctgtccacgtggaccgggggtgacatcgcacgtccatctgccaggacccctgcgtccaaattccgagacatggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagggtgggggccactcaccgacccgacactctcaccccccgacctggctgagtgcgaccaccacttgatgtctgaggataccttccatctcaacctacctcgagtggcgagtccagacaccatcaccacgcagagaggggaggtgggaggacagttagacccctgggtgggccctgccgtgggcaaggatacccggtggcttctggcacctggcgggctggtgacctttttaatccaggccccatcagcatcccacgatcggccttggtaaccgccgcggtagatcatttttatcccgccagggagtgtgatgcaggaagaccacatgcgctcctggcttttaaacctgttcctgactgttctcttactgccgaaacccttgactgttatctcggactttgcaggagttcctttccctccgaacgctgctccatgcacaggaaaagggcattttgtacaatggggacttcccgggaacgcttgctcttaagtaccagaagccggcggagctctggctttcgtgtttttggttttctccttcccaaggcagctggattgaaaaaacaaaacaaaacaaaaaaacccaggggcgtcagtcgatattcccagggccgcttctcctgcagtctgtggagcgtccttgtccccgccgccggaatgaatgagcattctgcagcccgatgtccctgtcccctcctcgccgggccattctgattggacctggcccagtgcaatctgtccagacaagccctgcttgctggaaaactgccacaagcacaattgatctctttttatcgccattccaggggcctcaggtcctactggggaaacttcctataccggagctccagtttctcttaagctgcccaatttcacagagtacaaaatagttgtaggggaaatcaaggtgaaggatctgtccgacagtcaagacggatccacagtaatctttcggtctccttaaactaccaccctcgctgccacccaccccaagctgctgccgcctcaccttccttgaaatttctcagcgggagtctcctcactgccactaaaatccacccagcccactaactgaggagctagtgttaatccagagaaccccccgcaatgtgcttccgagattcagactgcttcattgggaagtatgatttgttcctttctggaattgggctccgtggtggcggcggcacttcaagcaaagacagtttcttgcaagctccagtagctccgcgtgtctcatttgccaggaagatgggttcccacgtagcaaatcgtacattgtgccctgtagctccttagctagttagctcacaagccgtgttttatgactaatccttaataactatggtaaataactgtgactgtggggtttttaatctcttgtcattctcatccaaaagtgaccagcataccagttcttgcaataagatattaccctcagaatattaagcacattattgtagagaaaaaaaaatatgtgtacacatatgaacgcacaacatgcacattcatcctcacatgtggcacgtaaggtctcatttgatattgtgtaggaaatctgaagccttttcctgaggtcatctgtaaaatagtctcattgccaaggcatccccagtgccagctggtgaatccatgatcaaaatgcatacgtattgttaaatgataaggtttagaatgacaggaacccatcactgtgtctcatggtcccacttccccatctgtgtgtgaattcctttagactaagggcaggaagacttccagctttctctttgttcttcaatgtgaaactgagaccaagtctctctaagacaaatgcagtgtatttaatgtttgtaagcaattctaagtgagatgtttggcaagaaatcccctaactgatttccatccaaacctaccttatagagcacaatattaagtgttgtacaattactgtgagaactgtgaatatgtgtaacttttttttagtatttgcccggggggaaaaagatattgtattatcatatatgcttttttgcaataaggatttattctcagaacaccaagtaaatctatctctatataaaaaatatatgtaatatatacatattcaaagtatatacagagcctgttttaaaaaatacagtattatttagtaaaattatctgttctatggaccaaatgtaaaatatttataaatgaagatgcattttaaatgtctataaatggtgtcataactagagcacgggcgttatgtaagtttctaagaatttagaggataaataataaaggttctatgatatacaa

SEQ ID NO: 18 - DNA, 6015 bp, Homo sapiens
  source 1..6015: mol_type = genomic DNA
ctctctctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagagatgtacggggaccctctatgcaccgaacccaatacgttcattccccgtatgatcgtcctggttggaaccctcggttctgcatcatctcggggaaccagctgctcatgctggatgaggatgagatacaccccctactgatccgggaccggaggagcgagtccagtcgcaacaaactgctgagacgcacagtctccgtgccggtggaggggcggccccacggcgagcatgaataccacttgggtcgctcgaggaggaagagtgtcccaggggggaagcagtacagcatggagggtgcccctgctgcgcccttccggccctcgcaaggcttcctgagccgacggctaaaaagctccatcaaacgaacgaagtcacaacccaaacttgaccggaccagcagctttcgccagatcctgcctcgcttccgaagtgctgaccatgaccgggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtaacaacttcatcaggaacaaaatgctttgcctgtcggtctgcggccgaaagagacaaatggattgagaatctgcagcgggcagtaaagcccaacaaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggacttcctttcagacatggccatgtctgaggtagaccggttcatggaacgggagcacctcatattccgcgagaacacgcttgccactaaagccatagaagagtatatgagactgattggtcagaaatacctcaaggatgccattggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactgcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacagctccatcgaccttcagtccttcatggctcgaggcctcaacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggtgaaggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcaggagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttaggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttctttta

SEQ ID NO: 19 - DNA, 5966 bp, Homo sapiens
  source 1..5966: mol_type = genomic DNA
ctctctctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagagatgtacggggaccctctatgcaccgaacccaatacgttcattccccgtatgatcgtcctggttggaaccctcggttctgcatcatctcggggaaccagctgctcatgctggatgaggatgagatacaccccctactgatccgggaccggaggagcgagtccagtcgcaacaaactgctgagacgcacagtctccgtgccggtggaggggcggccccacggcgagcatgaataccacttgggtcgctcgaggaggaagagtgtcccaggggggaagcagtacagcatggagggtgcccctgctgcgcccttccggccctcgcaaggcttcctgagccgacggctaaaaagctccatcaaacgaacgaagtcacaacccaaacttgaccggaccagcagctttcgccagatcctgcctcgcttccgaagtgctgaccatgaccgggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtaacaacttcatcaggaacaaaatgctttgcctgtcggtctgcggccgaaagagacaaatggattgagaatctgcagcgggcagtaaagcccaacaaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggacttcctttcagacatggccatgtctgaggtagaccggttcatggaacgggagcacctcatattccgcgagaacacgcttgccactaaagccatagaagagtatatgagactgattggtcagaaatacctcaaggatgccattggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactgcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcagagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttaggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttctttta